(19) United States
(12) Patent Application Publication
(10) Pub. No.: US 2010/0130597 A1
(43) Pub. Date: May 27, 2010

(54) DNA-PKCS MODULATES ENERGY REGULATION AND BRAIN FUNCTION

(75) Inventors: Jay Hang Chung, Bethesda, MD (US); Myung Kyung Kim, Bethesda, MD (US); Sung Jun Park, Potomac, MD (US)

Correspondence Address: NIH-OTT, 1560 Broadway, Suite 1200, Denver, CO, 80238, US

(73) Assignee: The United State of America, as represented by the Secretary of the Dept of Health and Human Service, Rockville, MD (US)

(21) Appl. No.: 12/667,840

(22) PCT Filed: Jul. 3, 2008
(86) PCT No.: PCT/US08/08234

§ 371 Date: Feb. 1, 2010

Related U.S. Application Data
(60) Provisional application No. 60/958,714, filed on Jul. 6, 2007.

(51) Int. Cl. A61K 31/7088; A61K 31/535; A61K 31/5377; A61K 31/5513

(52) U.S. Cl. 514/44 R; 514/231.2; 514/233.5; 514/235.8; 514/218

(57) ABSTRACT

The invention relates to new functions of the DNA-PKcs gene product in energy metabolism, brain function and physical fitness.

This application claims benefit of the filing date of U.S. Provisional Patent Application Ser. No. 60/958,714 filed Jul. 6, 2007, the contents of which are specifically incorporated herein in their entirety.

### FIELD OF THE INVENTION

The present invention relates to novel functions of DNA-PKcs in energy regulation and brain function that are not lymphocyte-related. The present invention provides a method comprising suppressing activities of DNA-PKcs, mTOR, IKK and enhancing AMP-activated protein kinase (AMPK) and LKB1 kinase activities with DNA-PKcs inhibitors/antagonists or DNA-PKcs RNAi, without imposing calorie restriction. The present invention is also concerned with a method for preventing or treating various diseases, for example, metabolic disorders, aging-related physical decline, ischemic-reperfusion diseases, stroke, injury, inflammatory diseases, neurodegenerative diseases, other degenerative diseases, anxiety, depression, memory loss, cognitive disorders, mitochondrial diseases and eating disorders, described in this invention using DNA-PKcs inhibitors/antagonists or DNA-PKcs RNAi.

### BACKGROUND OF THE INVENTION

Among the physiological changes that occur with obesity and aging, the decline in physical fitness is most dramatic. As a result, the very people who will benefit most from physical exercise have a diminished capacity for it. Thus, an estimated 129.6 million American adults, or 64.5%, are overweight or obese {Ogden et al., J. Am. Med. Assoc. 295: 1549-55 (2006). Since 1980, the number of obese adults has doubled, and the number of obese children has tripled in the United States. Increased caloric intake and sedentary lifestyle are the main contributing factors to this trend. The prevalence of obesity also increases with age, and the number of people 65 or older is rapidly rising throughout the world. All these trends work together to create a vicious cycle that is difficult to break: obesity leads to decline in physical fitness, which leads to physical inactivity, which in turn further promotes obesity (Bluher, Science 299: 572-74 (2003)). Moreover, aging is associated with obesity as well as a decline in physical fitness, and at least in some mammals (e.g. rodents), obesity and physical inactivity may also affect aging (McCarter et al., Aging (Milano) 9: 73-79 (1997)).

The decline in physical fitness and endurance, which occurs with age and obesity, is partly due to the decline in mitochondrial function and content in skeletal muscle which also occurs with obesity (Kelley et al., Diabetes 51: 2944-50 (2002)) and aging (Petersen et al., Science 300: 1140-42 (2003); Short et al., Proc. Natl. Acad. Sci. USA 102: 5618-23 (2005)). Elderly subjects have approximately a 40% reduction in mitochondrial oxidative and phosphorylation activity, as assessed by in vivo ¹³C/³¹P NMR spectroscopy. The activity of rotenone-sensitive NADH:O(2) oxidoreductase, which reflects the overall activity of the mitochondrial respiratory chain, was reduced by approximately 20% in obese subjects and by approximately 40% in type 2 diabetic subjects as compared to healthy lean subjects (Kelley et al., Diabetes 51: 2944-50 (2002)).

Accordingly, a need exists for new compositions and methods to improve mitochondrial functioning, reduce obesity improve the health of middle-aged and elderly people.

### SUMMARY OF INVENTION

DNA-PKcs, the catalytic subunit of DNA-dependent protein kinase, is known for its function in nonhomologous end joining of DNA such as the V(D)J recombination that occurs in lymphocytes. In the absence of DNA-PKcs function, lymphocyte development is blocked, resulting in immunodeficiency. However, according to the present invention, DNA-PKcs has a previously unrecognized function in brain function and energy regulation that is not lymphocyte-related. DNA-PKcs deficient mice (SCID) have a significantly better memory than wild-type mice. SCID mice are also resistant to stress-induced binge eating of high fat foods. Moreover the decline in the expression of genes involved in mitochondrial biogenesis, thermogenesis and fat burning, which occurs with obesity and aging in wild-type littermates, does not occur in DNA-PKcs deficient mice. As a consequence, SCID mice have increased mitochondrial content and thermogenesis and are resistant to diet-induced obesity. One striking characteristic of middle-aged SCID mice is their exceptional physical fitness. Their muscles contain more mitochondria and approximately 40% more ATP than wild-type littermates. Significantly, middle-aged SCID mice are capable of running 2.5-3 times greater distances than wild-type littermates. Consistent with these findings, SCID mice are also more insulin sensitive.

According to the invention, administration of DNA-PKcs antagonists and inhibitors to mammals provides the same beneficial effects as genetic defects in the DNA-PKcs gene. Thus, for example, administration of the DNA-PKcs antagonist NU7026 decreased serum glucose levels as well as anxiety and depression levels in mice. In another example, administration of DNA-PKcs antagonist improved glucose response, prevented weight gain after high-fat diet, enhanced physical strength and stamina, and diminished anxiety/depression levels in mice.

Further according to the invention, the physical decline in obese and older mammals is not simple degeneration but is, at least partially, an active process driven by DNA-PKcs. Aging and obesity are also associated with increased inflammatory signaling. A number of diseases such as cancer, cardiovascular disease and diabetes, not to mention bona fide inflammatory diseases, are mediated by the IKK-NFκB-dependent inflammatory pathway. In the absence of DNA-PKcs, IKK-NFκB pathway is suppressed and at least in fat tissues, there is less inflammatory signaling. Surprisingly, DNA-PKcs also affects brain function. Moreover, SCID mice express higher levels of brain-derived neurotrophic factor (BDNF), which is associated with memory formation and suppression of anxiety and depression. Consistent with this, SCID mice have better memory and reduced anxiety compared to wild-type controls.

Thus, according to the invention inhibition of DNA-PKcs with DNA-PKcs antagonists, inhibitors, anti-sense RNA or with other means has unexpected utility in the treatment of a wide range of diseases and conditions.

One aspect of the invention is a method of inhibiting DNA-PKcs expression and/or activity in a mammal to increase mitochondrial numbers, to increase thermogenesis, to increase insulin sensitivity, to improve insulin signaling, to reduce blood glucose levels, to increase AMPK and PGC-1 alpha activities, to improve motor function, to improve memory and learning abilities, to reduce depression and anxiety, to reduce inflammatory signaling, and/or to increase eNOS, VEGF and BDNF expression,

the method comprising administering to the mammal a therapeutically effective amount of an inhibitor of DNA-PKcs activity

to reduce weight in the mammal, to increase mitochondrial numbers, to increase thermogenesis, to increase insulin sensitivity, to improve insulin signaling, to reduce blood glucose levels, to increase AMPK and PGC-1 alpha activities, to improve motor function, to improve memory and learning abilities, to reduce depression and anxiety, to reduce inflammatory signaling.

The methods of the invention are particularly beneficial for obese and/or middle-aged mammals.

The methods and compositions of the invention can also facilitate weight loss in a mammal. For example, mammals treated using the methods and compositions of the invention have reduced their weight by about 5% to about 20% relative to a control mammal that does not receive the inhibitor. The methods and compositions of the invention generally reduce the mammal's fat mass relative to a mammal that has not received the DNA-PKcs inhibitor. For example, mammals treated using the methods and compositions of the invention reduce their fat mass by about 5% to about 30% relative to a control mammal that does not receive the inhibitor.

The methods and compositions of the invention can also reduce serum triglycerides and/or serum leptin levels in a mammal. For example, after treatment using the methods and/or compositions of the invention, the serum triglycerides and/or serum leptin levels are reduced by about 5% to about 70% in the mammal relative to a control mammal that does not receive the inhibitor. These reductions are achieved even though the mammal does not significantly restrict calorie intake.

The methods and compositions of the invention can also increase mitochondrial numbers in a mammal by about two-fold to about three-fold relative to a control mammal that does not receive the inhibitor.

The methods and compositions of the invention can also increase thermogenesis in a mammal and, for example, increase the mammal's body temperature. In some embodiments, the mammal's body temperature increases by about 0.1° C. to about 1° C. after treatment using the methods and compositions of the invention relative to a control mammal that does not receive the inhibitor.

The methods and compositions of the invention can also increase oxygen usage in the mammal. For example, oxygen usage increases by about 5% to about 20% in a mammal treated using the methods and/or compositions of the invention relative to a control mammal that does not receive the inhibitor.

The methods and compositions of the invention can also increase AMPK, PPAR delta, CPT1b, UCP3, ERR alpha, VEGF, eNOS, PGC-1 alpha and/or PGC-1 beta expression in the mammal.

The methods and compositions of the invention can also improve a mammal's stamina during physical activity. For example, a mammal treated with the methods and/or compositions of the invention can run about 1.25 to about 3 times farther before exhaustion than a mammal that did not receive the inhibitor.

The methods and compositions of the invention can also increase ATP levels in mammals relative to a control mammal that does not receive the inhibitor. For example, ATP levels are about 5% to about 30% higher in mammals treated using the methods and compositions of the invention relative to control mammals that do not receive the inhibitor.

The methods and compositions of the invention can also reduce blood pressure in a mammal, for example, by about 10 mm Hg to about 30 mm Hg.

The methods and compositions of the invention can also increase insulin sensitivity and/or insulin signaling in the mammal. For example, insulin levels can be about 10% to about 50% lower in mammals treated using the methods and compositions of the invention relative to a control mammal that does not receive the inhibitor.

The methods and compositions of the invention can also reduce glucose levels the mammal after insulin treatment relative to a control mammal that does not receive the inhibitor. For example, after treatment using the methods and compositions, glucose levels can be about 5% to about 40% lower in the mammal after insulin treatment than in a control mammal that does not receive the inhibitor.

The methods and compositions of the invention can also improve memory and/or learning ability in a mammal. For example, when treated with the methods and compositions of the invention the mammal remembers where a target object is located better than a control mammal that did not receive the inhibitor. In some embodiments, the mammal remembers where a target object is located about 50% to about 100% better than a control mammal that did not receive the inhibitor.

The methods and compositions of the invention can also increase brain-derived neurotrophic factor (BDNF) and Sirt1 expression in the mammal. For example, when treated with the methods and/or compositions of the invention brain-derived neurotrophic factor (BDNF) or Sirt1 expression can be increased in the mammal by about 10% to about 40% relative to a control mammal that did not receive the inhibitor.

The methods and compositions of the invention can also reduce depression and/or anxiety in a mammal. Thus, the mammal engages in less anxiety-related food over-consumption when treated with the methods and compositions of the invention. For example, the mammal will generally consume about 20% to about 80% less high fat food after treatment with the compositions and methods of the invention.

The methods and compositions of the invention can also be used to make the mammal resistant to pain. For example, after treatment with the compositions and methods of the invention the mammal can resist pain about 10% to about 40% longer relative to a control mammal that did not receive the inhibitor.

The methods and compositions of the invention can also redeuce inflammation and/or inappropriate immune responses in a mammal, for example, by reducing macrophage numbers in a mammal by about 40% to about 80%. In some embodiments, the macrophage numbers are reduced in a mammal's adipose tissue.

The methods and compositions of the invention can also reduce the incidence of heart disease in a mammal, for example, in a mammal that is middle-aged or older.

The methods and compositions of the invention can also reduce the levels of reactive oxygen species in a mammal. For example, the levels of reactive oxygen species can be reduced in the mammal's heart by about 5% to about 50%.

The methods and compositions of the invention can also reduce a mammal's blood pressure is reduced. For example, in some embodiments, the mammal's blood pressure can be reduced by about 10 mm Hg to about 30 mm Hg.

The methods and compositions of the invention can also be used for treating or inhibiting a neurological disorder in a mammal. Examples of neurological disorders that can be used in the invention include Alzheimer's, Parkinson's, Huntington's disease, Amyotropic lateral sclerosis (ALS) and/or Friedreich ataxia (FRDA).

The nucleic acid that can inhibit the expression and/or translation of DNA-PKcs can hybridize to a nucleic acid having SEQ ID NO:2 under physiological conditions. In some embodiments, the nucleic acid that can inhibit the expression and/or translation of DNA-PKcs can hybridize to a nucleic acid having SEQ ID NO:2 under stringent hybridization conditions. Examples of nucleic acids that can inhibit the expression and/or translation of DNA-PKcs include small interfering RNAs (siRNAs) or ribozymes.

For example, the DNA-PKcs inhibitor used in the methods and compositions of the invention can be one or more compounds, each being a compound of formula I:

R₁—Ar—R₂(R₃)ₙ I

wherein:

R₁ is a hydrogen, lower alkoxy, cycloaryl, cycloheteroaryl, cycloalkyl or cycloheteroalkyl, wherein the cycloaryl, cycloheteroaryl, cycloalkyl and cycloheteroalkyl can optionally be substituted with one to four substituents selected from the group consisting of halo, hydroxy, lower alkyl, lower alkoxy, cyano, aryl, and heteroaryl;

Ar is cycloaryl or cycloheteroaryl that can optionally be substituted with one or two oxy (═O) or thio (═S or —SH) groups;

R₂ is cycloheteroaryl or cycloheteroalkyl;

R₃ is halo, lower alkyl, lower alkoxy, cyano, aryl, and heteroaryl; and

n is an integer of 0-3.

In some embodiments, the R₁ is hydrogen, or any of the following:

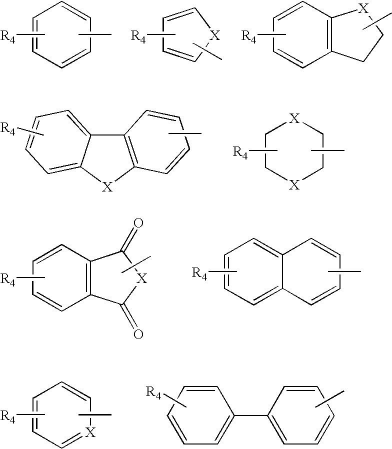

wherein X is a heteroatom, R₄ is hydrogen, halo, hydroxy, lower alkyl, lower alkoxy, cyano, aryl, and heteroaryl.

For example, the Ar moiety can be selected from the group consisting of:

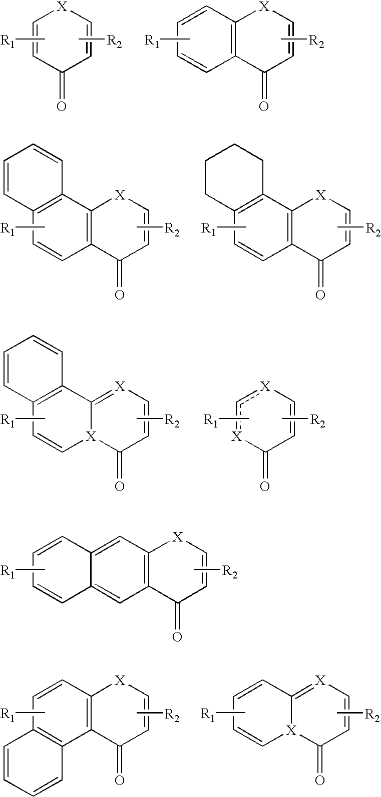

wherein X is a heteroatom and R₁ and R₂ are as defined herein.

The R₂ moiety can be selected from the group consisting of:

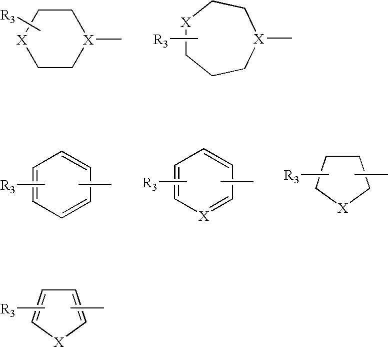

wherein R₃ is halo, lower alkyl, lower alkoxy, cyano, aryl, and heteroaryl.

In some embodiments the inhibitor is one or more of the compounds of formula II:

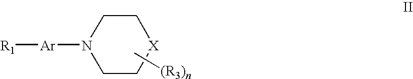

wherein R₁, Ar, R₃ and n are as defined above, and X is a heteroatom selected from the group consisting of O, NH or S.

Examples of inhibitors that can be used in the methods and compositions of the invention include a compound or a combination of compounds having the following structures:

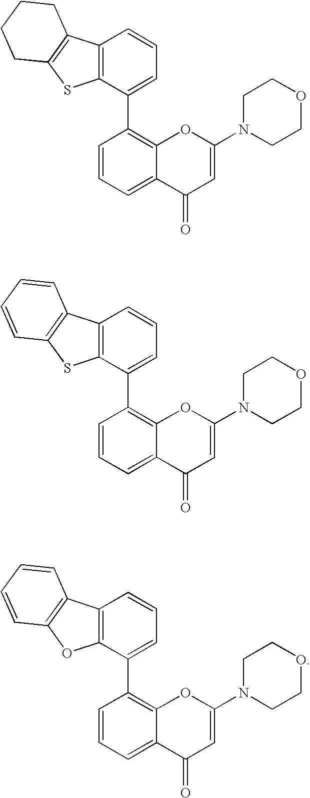

Other examples of inhibitors that can be used in the methods and compositions of the invention can be found in throughout the application. For example, the following compounds can be used in the methods and compositions of the invention: NU7026 (2-(morpholin-4-yl)-benzo[h]chomen-4-one), Euk-134, Manganese (III) tetrakis(4-benzoic acid)porphyrin (MnTBAP), 2,4-dinitrophenol (DNP), a nucleic acid that can inhibit the expression and/or translation of DNA-PKcs, a chromen-4-one compound or any combination thereof.

In some embodiments, the inhibitor is combined with resveratrol, metformin, thiazolidinediones (TZD), Epigallocatechin gallate (EGCG), IC60211 (2-hydroxy-4-morpholin-4-yl-benzaldehyde), IC86621 (a methyl ketone derivative of IC6021 1), IC486154, IC87102, IC87361, Wortmannin, LY294002, or any combination thereof.

### BRIEF DESCRIPTION OF THE FIGURES

In all figures, *, **, and *** indicate p<0.05, p<0.005 and p<0.0005, respectively, unless otherwise indicated.

FIG. 1 shows that DNA-PKcs is activated by H₂O₂ and inhibited by superoxide dismutase mimetic Euk-134.

FIG. 2 shows the reactive oxygen species levels in MCF-7 cells treated with varying concentrations of glucose and/or with Euk-134. *p<0.05 compared to the ROS level in cells exposed to 25 mM glucose. As illustrated, levels of reactive oxygen species increase as the concentration of glucose increases. Euk-134 is able to reduce the level of reactive oxygen species.

FIG. 3 illustrates that DNA-PKcs is increasingly activated (phosphorylated) by increasing concentrations of glucose.

FIG. 4 shows suppression of reactive oxygen species production with DNP, Euk-134, MnTBAP, Resveratrol and NU7026. *p<0.05, **p<0.005, ***p<0.0005 compared to the control (no modulating compound added). The relative intensity is a measure of the relative levels of reactive oxygen species.

FIGS. 5A and 5B illustrate inhibition of DNA-PKcs by a variety of agents. FIG. 5A shows suppression of DNA-PKcs with DNP, Euk-134, and MnTBAP in the presence of 25 mM glucose. FIG. 5B shows suppression of DNA-PKcs with metformin, Resveratrol and Euk-134 in the presence of 25 mM glucose.

FIG. 6A-6D illustrate that glucose metabolism does not induce DNA double strand breaks (DSB). DSB were detected by immunostaining with antibodies directed against phosphorylated histone H2AX (“γ-H2AX”), which recruits MDC1, 53BP1, and BRCA1 to chromatin near double-strand breaks and facilitates efficient repair of the break. FIGS. 6A and 6B show that the number of γ-H2AX-positive loci in cells exposed to 2 mM and 25 mM glucose, respectively, is not substantially different. These results are graphically summarized in FIG. 6C. In contrast, the number of γ-H2AX-positive loci substantially increases in cells exposed to ionizing radiation (FIG. 6D).

FIGS. 7A and 7B illustrate DNA-PKcs activity in soleus muscle samples biopsied from calorie restricted (CR) rhesus monkeys compared to those from monkeys fed ad libitum. FIG. 7A shows an immunoblot of soleus monkey muscle samples where activated DNA-PKcs was detected using a phospho-specific antibody capable of detecting activated monkey DNA-PKcs. The relative amount of phosphorylated DNA-PKcs from several monkeys is graphically summarized in FIG. 7B, where this quantification of DNA-PKcs activity indicates that calorie restriction significantly reduces DNA-PKcs activation (**, p=0.002).

FIG. 8 shows the levels of DNA-PKcs protein in skeletal muscle of lean 3 month- and 18 month-old mice as well as obese 6 month-old mice. Obesity tends to increase DNA-PKcs activity.

FIG. 9 shows growth curves of wild-type (WT, black symbols) and SCID (white symbols) littermates on a medium fat diet (MFD; circles; n=8 per genotype) and on a high fat diet (HFD; squares; n=7 per genotype). The SCID mice tend to have lower body weight.

FIG. 10 illustrates fat mass index (total fat mass/body weight) as measured by NMR spectroscopy for WT and SCID mice fed a medium fat diet (circles) and a high fat diet (squares). As illustrated, the wild type mice tend to have greater fat mass index.

FIGS. 11A to 11D illustrate the fat cell size of WT and SCID mice. FIGS. 11A and 11B show tissue sections illustrating the fat cell size of wild type (WT; FIG. 11A) and SCID (FIG. 11B) mice. The grams of mesenteric fat observed in mice maintained on a high fat diet for three months and treated with vehicle (control) or with a DNA-PKcs inhibitor (Compound 36 (Cpd 36)) is graphically summarized in FIGS. 11C and 11D, respectively.

FIG. 12A-12F show the relative mRNA levels of PGC-1α (FIG. 12A), PGC-1β (FIG. 12B), PPAR-delta (FIG. 12C), CPT1b (FIG. 12D), UCP1 (FIG. 12E) and ERRa (FIG. 12F), measured by real-time PCR in BAT from lean (L, 3-4 months old, RCD fed), obese (Ob, HFD fed) and middle-aged (MA, 14 months old) wild type (cross-hatched bars) and SCID (white bars) mice (n=3-4 per genotype).

FIGS. 13A to 13D graphically illustrate relative mRNA levels of PGC-1α (FIG. 13A), PPARδ (FIG. 13B), PGC-1β (FIG. 13C) and ERRα (FIG. 13D) in white adipose tissue (WAT) (n=3-4 per genotype) measured by real-time PCR.

FIGS. 14A to 14F show the relative mRNA levels of PGC-1α (FIG. 14A), PGC-1β (FIG. 14B), PPARδ (FIG. 14C), CPT1b (FIG. 14D), UCP3 (FIG. 14E) and ERRα (FIG. 14F) measured by real-time PCR in skeletal muscle from lean (L), obese (Ob) and middle-aged (MA) wild type (cross-hatched bars) and SCID (white bars) mice (n=3-4 per genotype).

FIG. 15 shows the relative amounts of mitochondrial DNA in skeletal muscle of wild type (cross-hatched bars) and SCID (white bars) mice.

FIG. 16 shows the distance (in meters) 4, 7 and 14 months old wild type (cross-hatched bars) and SCID (white bars) mice ran on the treadmill before exhaustion. For 4 months old, n=8; 7 months old, n=6; 14 months old, n=8, for each genotype.

FIGS. 17A to 17C illustrate the running endurance during three consecutive days of treadmill running before exhaustion for lean, obese and middle-aged WT and SCID mice, respectively. *,p<0.05; **,p<0.01.

FIG. 18 shows the AMPK activity in middle-aged WT and SCID tissues (skeletal muscle, white adipose tissue (WAT) and liver) and phosphorylation of AMPK substrate ACC1.

FIGS. 19A and 19B illustrate the level of ATP and ADP/ATP ratios, respectively, in skeletal muscle of middle-aged (14 month) wild type (cross-hatched bars) and SCID (open bars) mice.

FIGS. 20A and 20B show that the DNA-PKcs inhibitor Nu7026 activates AMPK activity in MCF7 cells without changing energy status. FIG. 20A shows that while increased glucose increases ATP percentage, Nu7026 does not affect ATP levels. FIG. 20B shows that Nu7026 activates AMPK activity in MCF7 cells.

FIGS. 21A and 21B illustrate the effects of knock-down of DNA-PKcs in 3T3-L1 adipocytes with DNA-PKcs-specific (PK) RNAi. DNA-PKcs siRNA activates AMPK and induces expression of PGC-1α, ERRα and CPT1b mRNA (FIG. 21A). As shown in FIG. 21B, an interfering RNA (RNAi) that is specific for DNA-PKcs reduces DNA-PKcs expression, whereas an RNA with a scrambled (S) sequence did not inhibit DNA-PKcs expression and was used as a control.

FIGS. 22A and 22B illustrate the fasting glucose levels and plasma insulin levels, respectively, of obese and middle-aged wild type (cross-hatched bars) and SCID (open bars) mice.

FIGS. 23A and 23B provide the results of an insulin tolerance test of overnight fasted mice at 6 months of age maintained on a breeder (regular chow diet, RCD, FIG. 23A) or a high-fat diet (n=8-12, FIG. 23B). The glucose levels after insulin injection are shown. (*,p<0.05; **,p<0.01, filled circles, Wild type; open circles, SCID mice)

FIGS. 24A and 24B show the AKT activity in white adipose tissue (Fat), liver and skeletal muscle of wild type and SCID mice that were maintained on a low fat (FIG. 24A) and high fat (FIG. 24B).

FIG. 25A-C show the relative mRNA levels of eNOS in muscle (FIG. 25A), VEGF in muscle (FIG. 25B) and SIRT1 in brown adipose tissue (FIG. 25C) in WT and SCID tissues as measured by real-time PCR.

FIG. 26 shows immunohistochemical detection of a macrophage-specific antigen in white adipose tissues (WAT) of WT and SCID mice.

FIG. 27A-C show the relative mRNA expression levels of IkBα in muscle (FIG. 27A), CCL2 in white adipose tissue (FIG. 27B) and CD68 in white adipose tissue (FIG. 27C) in wild type (cross-hatched bars) and SCID (open bars) tissues as measured by real-time PCR.

FIG. 28A-E illustrate the results from an elevated plus-maze test of WT and SCID mice after feeding breeder diet for 10 months. The elevated plus-maze is used to determine the rodent's response to a potentially dangerous environment and anxiety-related behavior is measured by the degree to which the rodent avoids the unenclosed arms of the maze. As illustrated, reduced anxiety-related behavior was observed in SCID mice. FIG. 28A shows that SCID mice spend significantly more time in open arms of the maze. Mice generally avoid the open arms because of their fear of open space and height. FIG. 28B shows that SCID mice spend more time in the center of the maze, which is an exposed position that mice generally avoid. FIG. 28C shows that SCID mice spend significantly less time in closed arms of the maze. FIG. 28D shows that SCID mice enter the open arms of the maze more frequently than wild type mice. FIG. 28E shows that SCID and wild type mice enter the closed arms of the maze with approximately the same frequency.

FIG. 29A-E show the results of a Light/Dark compartment test of WT and SCID mice after feeding breeder diet (medium fat diet) for 10 months. In the light-dark test, increased activity in the light compartment indicates decreased anxiety. FIG. 29A shows that SCID mice spend significantly less time in the dark chamber. FIG. 28B shows that SCID mice enter the dark chamber less frequently than wild type mice. FIG. 29C shows that SCID mice spend significantly more time in the light chamber. FIG. 29D shows that SCID mice enter the light chamber more quickly than wild type mice. FIG. 29E shows that SCID and wild type mice enter the dark chamber at approximately the same frequency. Reduced anxiety-related behavior was therefore observed in SCID mice.

FIG. 30 shows the pain tolerance of WT (open bars) and SCID (cross-hatched bars) mice as measured by the latency of the mice on a hot plate at 52° C. The abbreviations employed are as follows: LF (low fat diet), BR (breeder diet or medium fat diet, MFD), HF (high fat diet). *p<0.05, **p<0.005, ***p<0.0005 SCID compared to WT.

FIGS. 31A to 31B show the food intake of 2-3 months-old WT and SCID mice group-housed with littermates (4-5 mice/cage). FIG. 31A shows the amount of food in grams consumed per mouse per day. FIG. 31B shows the amount of food in grams consumed per gram of mouse per day. The abbreviations employed are as follows: LF (low fat diet), BR (breeder diet or medium fat diet, MFD), HF (high fat diet). *p<0.05, **p<0.005, ***p<0.0005 SCID compared to WT.

FIG. 32 shows the amount breeder diet (medium fat diet) and high-fat diet (HF) consumed after isolation of WT and SCID mice. Previously group-housed WT ad SCID mice were isolated (one per cage) and fed with HF or breeder diet for the indicated days.

FIGS. 33A and 33B show results of an elevated plus-maze test of WT and SCID mice with or without intraperitoneal injection of Zofran (FIG. 33A, no treatment; FIG. 33B, Zofran treatment).

FIG. 34A-D show the results of a Morris water maze test of 14 month old WT and SCID mice, which tests how well mice remember where a submerged platform is in a water tank. FIG. 34A shows that SCID mice consistently locate the submerged platform faster than wild type mice. FIG. 34B shows that SCID mice consistently spend more time in the quadrant of the tank that contains the submerged platform (the target). FIG. 34C shows that SCID and wild type mice move approximately the same distance. FIG. 34D shows that SCID mice consistently locate the submerged platform faster than wild type mice.

FIG. 35A-C show the results of an object recognition test of 14 month old WT and SCID mice.

FIG. 36A-C show reactive oxygen species (ROS) levels in muscle, white adipose tissue and heart tissues, respectively, of WT and SCID mice. The abbreviations employed are as follows: Lean (L), obese (Ob) and middle-aged (MA, 14 month or 18 month old).

FIG. 37 illustrates lipid peroxidation levels in white adipose tissues of WT and SCID mice.

FIG. 38 illustrates reactive oxygen species (ROS) levels in tissues from ob/ob mice that are either not treated with Euk-134 (control; cross-hatched bars) or treated with Euk-134 (open bars).

FIG. 39 shows that SCID mice (open circles) run greater distances than wild type mice (closed circles) on a treadmill test.

FIG. 40A-B illustrate the effect of DNA-PK inhibitor Compound 36 (Cpd 36) treatment for three months on fed plasma glucose levels in HFD obese C57BL/6J mice (high fat diet for three months, FIG. 40A) or middle-aged (breeder diet for 13 months, FIG. 40B) C57BL/6J mice. The blood glucose levels in mg/dl of mice treated with Cpd 36 (cross-hatched bars) are compared with control mice that were not treated with Cpd 36 (open bars).

FIG. 41A-B illustrate improvement in insulin tolerance test (ITT) and glucose tolerance test (GTT) in middle-aged mice treated with Cpd36 (open circle) compared to control mice that were not treated with Cpd36 (filled circles). FIG. 41A shows the percent glucose in the blood of mice as a function of time. FIG. 41B shows the blood glucose levels in mg/dl of mice as a function of time.

FIG. 42A-B illustrate improved glucose responses in insulin sensitivity tests and glucose tolerance tests performed on high-fat diet mice treated with Cpd36.

FIG. 43A-B show the body weight (FIG. 43A) and the weight gain (FIG. 43B) of mice treated with Cpd36.

FIG. 44 shows fat mass and lean mass of mice fed a high fat diet after Cpd36 treatment. As indicated, the mice treated with Cpd36 have somewhat less fat mass and somewhat more lean mass.

FIG. 45 illustrates a dramatic improvement in physical endurance in Cpd36-treated mice maintained on a high fat diet (HFD) for three months.

FIG. 46A-B illustrate serum lactate levels of mice treated with Cpd36 (FIG. 46A) and cellular lactate level in differentiated C2C12 cells treated with Cpd36 (FIG. 46B).

FIG. 47A-D illustrate reduced anxiety/depression in mice after treatment with DNA-PKcs inhibitors. FIG. 47A shows that mice treated with the DNA-PKcs inhibitor Cpd36 spend less time in a closed arm of the elevated plus maze. FIG. 47B shows that mice treated with the DNA-PKcs inhibitor Cpd36 are quicker to enter the light chamber. FIG. 47C shows that mice treated with the DNA-PKcs inhibitor Cpd36 spend more time in the light compartment. FIG. 47D shows that mice treated with the DNA-PKcs inhibitor Cpd36 are more mobile than untreated mice.

FIG. 48A-E illustrate reduced anxiety/depression and pain sensation in mice treated with DNA-PKcs inhibitors. FIG. 48A shows that mice treated with the DNA-PKcs inhibitor Nu7026 spend less time in a closed arm of the elevated plus maze. FIG. 48B shows that mice treated with the DNA-PKcs inhibitor Nu7026 spend more time in the open arm of the elevated plus maze. FIG. 48C shows that mice treated with the DNA-PKcs inhibitor Nu7026 are quicker to enter the light chamber. FIG. 48D shows that mice treated with the DNA-PKcs inhibitor Nu7026 spend more time in the light compartment and less time in the dark compartment during the light/dark chamber test. FIG. 48E shows that mice treated with the DNA-PKcs inhibitor Nu7026 remain on a hot surface for longer periods of time.

FIG. 49A-C illustrate elevated Sirt1 and PGC-1α levels in C2C12 cells treated with DNA-PKcs inhibitors. FIG. 49A shows that Sirt1 levels increase upon treatment of C2C12 cells with NU7026 and Resveratrol. FIG. 49B shows that Sirt1 and PGC-1α levels are increased in NU7026-treated and Resveratrol-treated C2C12 cells. FIG. 49C graphically illustrates that the relative copy number of mitochondrial DNA increases when C2C12 myoblasts are treated with NU7026 and Resveratrol.

FIG. 50A-B illustrate that AMPK is activated in C2C12 cells treated with CamK inhibitor (STO609) and/or NU7026. FIG. 50A shows that AMPK is activated by NU7026. AMPK phosphorylation increased over time (0-16 hr). FIG. 50B shows that AMPK is still activated when both STO609 and the DNA-PKcs inhibitor, NU7026, are present.

FIG. 51A-B illustrate activation of LKB1 in cells treated with DNA-PKcs inhibitors and basal levels of LKB1 in DNA-PKcs-deficient SCID mice. FIG. 51A illustrates NU7026-induced and Resveratrol-induced LKB1 activation in C2C12 cells. FIG. 51B illustrates LKB1 basal activity in SCID tissues. Note that white adipose tissue of SCID mice have increased LKB1 activity.

FIG. 52A-B illustrate that LKB1 is required for NU7026-induced AMPK activation in cells. FIG. 52A shows that AMPK was activated in wild-type MEFs after the NU7026 treatment and was increased in DNA-PK knockout (DNA-PK KO) cells. FIG. 52B shows that loss of LKB1 function (LKB1 KO) in mouse embryonic fibroblasts (MEFs) leads to lower levels of AMPK activation.

FIG. 53 shows that loss of AMPKα1/α2 function (AMPK KO) suppresses activation of PGC-1α expression that would normally occur when cells are exposed to DNA-PKcs inhibitors (e.g., NU7026).

FIG. 54 shows an elevated cellular NAD/NADH ratio in C2C12 cells after Cpd36-treatment. Similar results were obtained when the C2C12 cells were treated with Resveratrol (data not shown).

FIG. 55 shows that DNA-PKcs is activated in old Rhesus monkeys (14-16 years), whereas young monkeys (1 to 1.5 years) exhibit little or no DNA-PKcs activation.

FIG. 56 shows that LKB1 is more active in younger monkeys and in calorie restricted monkeys than in aging Rhesus monkeys.

FIG. 57 shows a schematic diagram illustrating the Stress-Activated DNA-PKcs (SAD) pathways in obesity and aging-related disorders, which is further described in the application.

### DETAILED DESCRIPTION OF INVENTION

As described herein, DNA-PKcs has previously unrecognized functions in energy regulation and brain function that are not lymphocyte-related.

According to the invention, inhibition or loss of DNA-PKcs activity produces biochemical and physiological changes associated with longer lifespan, increased mitochondrial number and thermogenesis, increased insulin sensitivity and insulin signaling, reduced AKT activation, reduced blood glucose level, increased AMPK and PGC-1 alpha activity, improved motor function, memory and learning abilities, suppression of depression and anxiety, reduced inflammatory signaling, and increased eNOS, VEGF and BDNF expression. The health beneficial effects exerted in SCID mice are very similar to those of calorie restriction. Because of the enormous potential benefits of calorie restriction in health, it is critically important to develop calorie restriction mimetics that mimic the beneficial effects of calorie restriction.

Due to the far-reaching effects of DNA-PKcs inhibitors demonstrated herein, the methods of the invention can be used to treat a variety of diseases. These diseases and conditions include, but are not limited to, metabolic disorders such as type II diabetes, obesity, cardiovascular diseases and dyslipidemia, anxiety, depression, aging-related physical decline, memory loss, ischemic-reperfusion diseases, stroke, injury, inflammatory diseases, neurodegenerative diseases, eating disorders, mitochondrial diseases and other degenerative diseases.

### DNA-PKcs

The DNA-dependent protein kinase catalytic subunit (DNA-PKcs) is part of the DNA-dependent protein kinase (DNA-PK), which has previously been recognized as being involved in DNA double-stranded break repair and V(D)J recombination. DNA-PK is a trimeric complex consisting of DNA-PKcs (DNA-dependent protein kinase catalytic subunit), Ku70 and Ku80 that is activated by DNA-breaks. While DNA-PK is best known for its function in repair of DNA breaks that occur during V(D)J recombination in lymphocytes by non-homologous end joining, as described and demonstrated herein, DNA-PKcs has a much larger role in the health, aging and physical fitness of mammals.

The only currently identified stimuli that efficiently activate DNA-PK are DNA double stranded-breaks (DSBs). DNA-PK mediates the repair of DNA DSBs that occur during V(D)J recombination in lymphocytes (Blunt et al. Cell 80: 813-23 (1995)) through nonhomologous end joining (NHEJ) (Critchlow & Jackson, Trends Biochem. Sci. 23: 394-98 (1998)). As a result, DNA-PKcs^{-/-} mice (Taccioli et al. Immunity 9: 355-66 (1998); Gao et al. Immunity 9: 367-76 (1998)) and SCID (Severe Combined Immune Deficiency) mice (Bosma et al. Nature 301: 527-30 (1983)), which carry a nonsense mutation that truncates 83 amino acids from the C-terminus end of the kinase domain of DNA-PKcs (Blunt et al. Proc. Natl. Acad. Sci. USA 93: 10285-90 (1996)), have a block in lymphocyte development. Although DNA-PKcs is expressed ubiquitously, DNA-PKcs-deficient mice develop normally and DNA-PKcs-deficient fibroblasts grow well in culture. Fibroblasts deficient in other DNA repair proteins usually grow very poorly (Barlow et al. Cell 86: 159-71 (1996)). Since DNA-PKcs mediates non-homologous end-joining of DNA and is thought to be important for genetic stability, one would expect a significant increase in the incidence of tumors in SCID or DNA-PKcs^{-/-} mice. However, the incidence of lymphoma in DNA-PKcs^{-/-} mice is only slightly increased compared to wild-type mice. The incidence of lymphoma in mice increases more significantly only when both the DNA-PKcs and the tumor suppressor gene p53 are defective.

An example of an amino acid sequence for a human DNA-PKcs protein can be found in the National Center for Biotechnology Information (NCBI) database (http://www.ncbi.nlm.nih.gov/) as accession number AAB39925 (gi: 13570017). See ncbi.nlm.nih.gov. This amino acid sequence (SEQ ID NO:1) is as follows.

| | |
|---|---|
| 1 | MAGSGAGVRC SLLRLQETLS AADRCGAALA GHQLIRGLGQ |
| 41 | ECVLSSSPAV LALQTSLVFS RDFGLLVFVR KSLNSIEFRE |
| 81 | CREEILKFLC IFLEKMGQKI APYSVEIKNT CTSVYTKDRA |
| 121 | AKCKIPALDL LIKLLQTFRS SRLMDEFKIG ELFSKFYGEL |
| 161 | ALKKKIPDTV LEKVYELLGL LGEVHPSEMI NNAENLFRAF |
| 201 | LGELKTQMTS AVREPKLPVL AGCLKGLSSL LCNETKSMEE |
| 241 | DPQTSREIFN FVLKAIRPQI DLKRYAVPSA GLRLPALHAS |
| 281 | QESTCLLDNY VSLFEVLLKW CAHTNVELKK AALSALESFL |
| 321 | KQVSNMVAKN AEMHKNKLQY FMEQFYGIIR NVDSNNKELS |
| 361 | IAIRGYGLFA GPCKVINAKD VDFMYVELIQ RCKQMFLTQT |
| 401 | DTGDDRVYQM PSFLQSVASV LLYLDTVPEV YTPVLEHLVV |
| 441 | MQIDSFPQYS PKMQLVCCRA IVKVFLALAA KGPVLRNCIS |
| 481 | TVVHQGLIRI CSKPVVLPKG PESESEDHRA SGEVRTGKWK |
| 521 | VPTYKDYVDL FRHLLSSDQM MDSILADEAF FSVNSSSESL |
| 561 | NHLLYDEFVK SVLKIVEKLD LTLEIQTVGE QENGDEAPGV |
| 601 | WMIPTSDPAA NLHPAKPKDF SAFINLVEFC REILPEKQAE |
| 641 | FFEPWVYSFS YELILQSTRL PLISGFYKLL SITVRNAKKI |
| 681 | KYFEGVSPKS LKHSPEDPEK YSCFALFVKF GKEVAVKMKQ |
| 721 | YKDELLASCL TFLLSLPHNI IELDVRAYVP ALQMAFKLGL |
| 761 | SYTPLAEVGL NALEEWSIYI DRHVMQPYYK DILPCLDGYL |
| 801 | KTSALSDETK NNWEVSALSR AAQKGFNKVV LKHLKKTKNL |
| 841 | SSNEAISLEE IRIRVVQMLG SLGGQINKNL LTVTSSDEMM |
| 881 | KSYVAWDREK RLSFAVPFRE MKPVIFLDVF LPRVTELALT |
| 921 | ASDRQTKVAA CELLHSMVMF MLGKATQMPE GGQGAPPMYQ |
| 961 | LYKRTFPVLL RLACDVDQVT RQLYEPLVMQ LIHWFTNNKK |
| 1001 | FESQDTVALL EAILDGIVDP VDSTLRDFCG RCIREFLKWS |
| 1041 | IKQITPQQQE KSPVNTKSLF KRLYSLALHP NAFKRLGASL |
| 1081 | AFNNIYREFR EEESLVEQFV FEALVIYMES LALAHADEKS |
| 1121 | LGTIQQCCDA IDHLCRIIEK KHVSLNKAKK RRLPRGFPPS |
| 1161 | ASLCLLDLVK WLLAHCGRPQ TECRHKSIEL FYKFVPLLPG |
| 1201 | NRSPNLWLKD VLKEEGVSFL INTFEGGGCG QPSGILAQPT |
| 1241 | LLYLRGPFSL QATLCWLDLL LAALECYNTF TGERTVGALQ |
| 1281 | VLGTEAQSSL LKAVAFFLES IAMHDIIAAE KCFGTGAAGN |
| 1321 | RTSPQEGERY NYSKCTVVVR IMEFTTTLLN TSPEGWKLLK |
| 1361 | KDLCNTHLMR VLVQTLCEPA SIGFNIGDVQ VMAHLPDVCV |
| 1401 | NLMKALKMSP YKDILETHLR EKITAQSIEE LCAVNLYGPD |
| 1441 | AQVDRSRLAA VVSACKQLHR AGLLHNILPS QSTDLHHSVG |
| 1481 | TELLSLVYKG IAPGDERQCL PSLDLSCKQL ASGLLELAFA |
| 1521 | FGGLCERLVS LLLNPAVLST ASLGSSQGSV IHFSHGEYFY |
| 1561 | SLFSETINTE LLKNLDLAVL ELMQSSVDNT KMVSAVLNGM |
| 1601 | LDQSFRERAN QKHQGLKLAT TILQHWKKCD SWWAKDSPLE |
| 1641 | TKMAVLALLA KILQIDSSVS FNTSHGSFPE VFTTYISLLA |
| 1681 | DTKLDLHLKG QAVTLLPFFT SLTGGSLEEL RRVLEQLIVA |
| 1721 | HFPMQSREFP PGTPRFNNYV DCMKKFLDAL ELSQSPMLLE |
| 1761 | LMTEVLCREQ QHVMEELFQS SFRRIARRGS CVTQVGLLES |
| 1801 | VYEMFRKDDP RLSFTRQSFV DRSLLTLLWH CSLDALREFF |
| 1841 | STIVVDAIDV LKSRFTKLNE STFDTQITKK MGYYKILDVM |
| 1881 | YSRLPKDDVH AKESKINQVF HGSCITEGNE LTKTLIKLCY |
| 1921 | DAFTENMAGE NQLLERRRLY HCAAYNCAIS VICCVFNELK |
| 1961 | FYQGFLFSEK PEKNLLIFEN LIDLKRRYNF PVEVEVPMER |
| 2001 | KKKYIEIRKE AREAANGDSD GPSYMSSLSY LADSTLSEEM |
| 2041 | SQFDFSTGVQ SYSYSSQDPR PATGRFRRRE QRDPTVHDDV |
| 2081 | LELEMDELNR HECMAPLTAL VKHMHRSLGP PQGEEDSVPR |
| 2121 | DLPSWMKFLH GKLGNPIVPL NIRLFLAKLV INTEEVFRPY |
| 2161 | AKHWLSPLLQ LAASENNGGE GIHYMVVEIV ATILSWTGLA |
| 2201 | TPTGVPKDEV LANRLLNFLM KHVFHPKRAV FRHNLEIIKT |
| 2241 | LVECWKDCLS IPYRLIFEKF SGKDPNSKDN SVGIQLLGIV |
| 2281 | MANDLPPYDP QCGIQSSEYF QALVNNMSFV RYKEVYAAAA |
| 2321 | EVLGLILRYV MERKNILEES LCELVAKQLK QHQNTMEDKF |
| 2361 | IVCLNKVTKS FPPLADRFMN AVFELLPKFH GVLKTLCLEV |
| 2401 | VLCRVEGMTE LYFQLKSKDF VQVMRHRDDE RQKVCLDIIY |
| 2441 | KMMPKLKPVE LRELLNPVVE FVSHPSTTCR EQMYNILMWI |
| 2481 | HDNYRDPESE TDNDSQEIFK LAKDVLIQGL IDENPGLQLI |
| 2521 | IRNFWSHETR LPSNTLDRLL ALNSLYSPKI EVHFLSLATN |
| 2561 | FLLEMTSMSP DYPNPMFEHP LSECEFQEYT IDSDWRFRST |
| 2601 | VLTPMFVETQ ASQGTLQTRT QEGSLSARWP VAGQIRATQQ |
| 2641 | QHDFTLTQTA DGRSSFDWLT GSSTDPLVDH TSPSSDSLLF |
| 2681 | AHKRSERLQR APLKSVGPDF GKKRLGLPGD EVDNKVKGAA |
| 2721 | GRTDLLRLRR RFMRDQEKLS LMYARKGVAE QKREKEIKSE |
| 2761 | LKMKQDAQVV LYRSYRHGDL PDIQIKHSSL ITPLQAVAQR |
| 2801 | DPIIAKQLFS SLFSGILKEM DKFKTLSEKN NITQKLLQDF |
| 2841 | NRFLNTTFSF FPPFVSCIQD ISCQHAALLS LDPAAVSAGC |
| 2881 | LASLQQPVGI RLLEEALLRL LPAELPAKRV RGKARLPPDV |
| 2921 | LRWVELAKLY RSIGEYDVLR GIFTSEIGTK QITQSALLAE |
| 2961 | ARSDYSEAAK QYDEALNKQD WVDGEPTEAE KDFWELASLD |
| 3001 | CYNHLAEWKS LEYCSTASID SENPPDLNKI WSEPFYQETY |
| 3041 | LPYMIRSKLK LLLQGEADQS LLTFIDKAMH GELQKAILEL |
| 3081 | HYSQELSLLY LLQDDVDRAK YYIQNGIQSF MQNYSSIDVL |
| 3121 | LHQSRLTKLQ SVQALTEIQE FISFISKQGN LSSQVPLKRL |
| 3161 | LNTWTNRYPD AKMDPMNIWD DIITNRCFFL SKIEEKLTPL |
| 3201 | PEDNSMNVDQ DGDPSDRMEV QEQEEDISSL IRSCKFSMKM |
| 3241 | KMIDSARKQN NFSLAMKLLK ELHKESKTRD DWLVSWVQSY |
| 3281 | CRLSHCRSRS QGCSEQVLTV LKTVSLLDEN NVSSYLSKNI |
| 3321 | LAFRDQNILL GTTYRIIANA LSSEPACLAE IEEDKARRIL |
| 3361 | ELSGSSSEDS EKVIAGLYQR AFQHLSEAVQ AAEEEAQPPS |
| 3401 | WSCGPAAGVI DAYMTLADFC DQQLRKEEEN ASVIDSAELQ |
| 3441 | AYPALVVEKM LKALKLNSNE ARLKFPRLLQ IIERYPEETL |
| 3481 | SLMTKEISSV PCWQFISWIS HMVALLDKDQ AVAVQHSVEE |
| 3521 | ITDNYPQAIV YPFIISSESY SFKDTSTGHK NKEFVARIKS |
| 3561 | KLDQGGVIQD FINALDQLSN PELLFKDWSN DVRAELAKTP |
| 3601 | VNKKNIEKMY ERMYAALGDP KAPGLGAFRR KFIQTFGKEF |
| 3641 | DKHFGKGGSK LLRMKLSDFN DITNMLLLKM NKDSKPPGNL |
| 3681 | KECSPWMSDF KVEFLRNELE IPGQYDGRGK PLPEYHVRIA |
| 3721 | GFDERVTVMA SLRRPKRIII RGHDEREHPF LVKGGEDLRQ |
| 3761 | DQRVEQLFQV MNGILAQDSA CSQRALQLRT YSVVPMTSRL |
| 3801 | GLIEWLENTV TLKDLLLNTM SQEEKAAYLS DPRAPPCEYK |
| 3841 | DWLTKMSGKH DVGAYMLMYK GANRTETVTS FRKRESKVPA |
| 3881 | DLLKRAFVRM STSPEAFLAL RSHFASSHAL ICISHWILGI |
| 3921 | GDRHLNNFMV AMETGGVIGI DFGHAFGSAT QFLPVPELMP |
| 3961 | FRLTRQFINL MLPMKETGLM YSIMVHALRA FRSDPGLLTN |
| 4001 | TMDVFVKEPS FDWKNFEQKM LKKGGSWIQE INVAEKNWYP |
| 4041 | RQKICYAKRK LAGANPAVIT CDELLLGHEK APAFRDYVAV |
| 4081 | ARGSKDHNIR AQEPESGLSE ETQVKCLMDQ ATDPNILGRT |
| 4121 | WEGWEPWM |

A nucleotide sequence for this DNA-PKcs polypeptide is provided by the NCBI database as accession number U47077 (gi: 13570016), which is shown below for easy reference (SEQ ID NO:2).

| | |
|---|---|
| 1 | GGGGCATTTC CGGGTCCGGG CCGAGCGGGC GCACGCGCGG |
| 41 | GAGCGGGACT CGGCGGCATG GCGGGCTCCG GAGCCGGTGT |
| 81 | GCGTTGCTCC CTGCTGCGGC TGCAGGAGAC CTTGTCCGCT |
| 121 | GCGGACCGCT GCGGTGCTGC CCTGGCCGGT CATCAACTGA |
| 161 | TCCGCGGCCT GGGGCAGGAA TGCGTCCTGA GCAGCAGCCC |
| 201 | CGCGGTGCTG GCATTACAGA CATCTTTAGT TTTTTCCAGA |
| 241 | GATTTCGGTT TGCTTGTATT TGTCCGGAAG TCACTCAACA |
| 281 | GTATTGAATT TCGTGAATGT AGAGAAGAAA TCCTAAAGTT |
| 321 | TTTATGTATT TTCTTAGAAA AAATGGGCCA GAAGATCGCA |
| 361 | CCTTACTCTG TTGAAATTAA GAACACTTGT ACCAGTGTTT |
| 401 | ATACAAAAGA TAGAGCTGCT AAATGTAAAA TTCCAGCCCT |
| 441 | GGACCTTCTT ATTAAGTTAC TTCAGACTTT TAGAAGTTCT |
| 481 | AGACTCATGG ATGAATTTAA AATTGGAGAA TTATTTAGTA |
| 521 | AATTCTATGG AGAACTTGCA TTGAAAAAAA AAATACCAGA |
| 561 | TACAGTTTTA GAAAAAGTAT ATGAGCTCCT AGGATTATTG |
| 601 | GGTGAAGTTC ATCCTAGTGA GATGATAAAT AATGCAGAAA |
| 641 | ACCTGTTCCG CGCTTTTCTG GGTGAACTTA AGACCCAGAT |
| 681 | GACATCAGCA GTAAGAGAGC CCAAACTACC TGTTCTGGCA |
| 721 | GGATGTCTGA AGGGGTTGTC CTCACTTCTG TGCAACTTCA |
| 761 | CTAAGTCCAT GGAAGAAGAT CCCCAGACTT CAAGGGAGAT |
| 801 | TTTTAATTTT GTACTAAAGG CAATTCGTCC TCAGATTGAT |
| 841 | CTGAAGAGAT ATGCTGTGCC CTCAGCTGGC TTGCGCCTAT |
| 881 | TTGCCCTGCA TGCATCTCAG TTTAGCACCT GCCTTCTGGA |
| 921 | CAACTACGTG TCTCTATTTG AAGTCTTGTT AAAGTGGTGT |
| 961 | GCCCACACAA ATGTAGAATT GAAAAAAGCT GCACTTTCAG |
| 1001 | CCCTGGAATC CTTTCTGAAA CAGGTTTCTA ATATGGTGGC |
| 1041 | GAAAAATGCA GAAATGCATA AAAATAAACT GCAGTACTTT |
| 1081 | ATGGAGCAGT TTTATGGAAT CATCAGAAAT GTGGATTCGA |
| 1121 | ACAACAAGGA GTTATCTATT GCTATCCGTG GATATGGACT |
| 1161 | TTTTGCAGGA CCGTGCAAGG TTATAAACGC AAAAGATGTT |
| 1201 | GACTTCATGT ACGTTGAGCT CATTCAGCGC TGCAAGCAGA |
| 1241 | TGTTCCTCAC CCAGACAGAC ACTGGTGACG ACCGTGTTTA |
| 1281 | TCAGATGCCA AGCTTCCTCC AGTCTGTTGC AAGCGTCTTG |
| 1321 | CTGTACCTTG ACACAGTTCC TGAGGTGTAT ACTCCAGTTC |
| 1361 | TGGAGCACCT CGTGGTGATG CAGATAGACA GTTTCCCACA |
| 1401 | GTACAGTCCA AAAATGCAGC TGGTGTGTTG CAGAGCCATA |
| 1441 | GTGAAGGTGT TCCTAGCTTT GGCAGCAAAA GGGCCAGTTC |
| 1481 | TCAGGAATTG CATTAGTACT GTGGTGCATC AGGGTTTAAT |
| 1521 | CAGAATATGT TCTAAACCAG TGGTCCTTCC AAAGGGCCCT |
| 1561 | GAGTCTGAAT CTGAAGACCA CCGTGCTTCA GGGGAAGTCA |
| 1601 | GAACTGGCAA ATGGAAGGTG CCCACATACA AAGACTACGT |
| 1641 | GGATCTCTTC AGACATCTCC TGAGCTCTGA CCAGATGATG |
| 1681 | GATTCTATTT TAGCAGATGA AGCATTTTTC TCTGTGAATT |
| 1721 | CCTCCAGTGA AAGTCTGAAT CATTTACTTT ATGATGAATT |
| 1761 | TGTAAAATCC GTTTTGAAGA TTGTTGAGAA ATTGGATCTT |
| 1801 | ACACTTGAAA TACAGACTGT TGGGGAACAA GAGAATGGAG |
| 1841 | ATGAGGCGCC TGGTGTTTGG ATGATCCCAA CTTCAGATCC |
| 1881 | AGCGGCTAAC TTGCATCCAG CTAAACCTAA AGATTTTTCG |
| 1921 | GCTTTCATTA ACCTGGTGGA ATTTTGCAGA GAGATTCTCC |
| 1961 | CTGAGAAACA AGCAGAATTT TTTGAACCAT GGGTGTACTC |
| 2001 | ATTTTCATAT GAATTAATTT TGCAATCTAC AAGGTTGCCC |
| 2041 | CTCATCAGTG GTTTCTACAA ATTGCTTTCT ATTACAGTAA |
| 2081 | GAAATGCCAA GAAAATAAAA TATTTCGAGG GAGTTAGTCC |
| 2121 | AAAGAGTCTG AAACACTCTC CTGAAGACCC AGAAAAGTAT |
| 2161 | TCTTGCTTTG CTTTATTTGT GAAATTTGGC AAAGAGGTGG |
| 2201 | CAGTTAAAAT GAAGCAGTAC AAAGATGAAC TTTTGGCCTC |
| 2241 | TTGTTTGACC TTTCTTCTGT CCTTGCCACA CAACATCATT |
| 2281 | GAACTCGATG TTAGAGCCTA CGTTCCTGCA CTGCAGATGG |
| 2321 | CTTTCAAACT GGGCCTGAGC TATACCCCCT TGGCAGAAGT |
| 2361 | AGGCCTGAAT GCTCTAGAAG AATGGTCAAT TTATATTGAC |
| 2401 | AGACATGTAA TGCAGCCTTA TTACAAAGAC ATTCTCCCCT |
| 2441 | GCCTGGATGG ATACCTGAAG ACTTCAGCCT TGTCAGATGA |
| 2481 | GACCAAGAAT AACTGGGAAG TGTCAGCTCT TTCTCGGGCT |
| 2521 | GCCCAGAAAG GATTTAATAA AGTGGTGTTA AAGCATCTGA |
| 2561 | AGAAGACAAA GAACCTTTCA TCAAACGAAG CAATATCCTT |
| 2601 | AGAAGAAATA AGAATTAGAG TAGTACAAAT GCTTGGATCT |
| 2641 | CTAGGAGGAC AAATAAACAA AAATCTTCTG ACAGTCACGT |
| 2681 | CCTCAGATGA GATGATGAAG AGCTATGTGG CCTGGGACAG |
| 2721 | AGAGAAGCGG CTGAGCTTTG CAGTGCCCTT TAGAGAGATG |
| 2761 | AAACCTGTCA TTTTCCTGGA TGTGTTCCTG CCTCGAGTCA |
| 2801 | CAGAATTAGC GCTCACAGCC AGTGACAGAC AAACTAAAGT |
| 2841 | TGCAGCCTGT GAACTTTTAC ATAGCATGGT TATGTTTATG |
| 2881 | TTGGGCAAAG CCACGCAGAT GCCAGAAGGG GGACAGGGAG |
| 2921 | CCCCACCCAT GTACCAGCTC TATAAGCGGA CGTTTCCTGT |
| 2961 | GCTGCTTCGA CTTGCGTGTG ATGTTGATCA GGTGACAAGG |
| 3001 | CAACTGTATG AGCCACTAGT TATGCAGCTG ATTCACTGGT |
| 3041 | TCACTAACAA CAAGAAATTT GAAAGTCAGG ATACTGTTGC |
| 3081 | CTTACTAGAA GCTATATTGG ATGGAATTGT GGACCCTGTT |
| 3121 | GACAGTACTT TAAGAGATTT TTGTGGTCGG TGTATTCGAG |
| 3161 | AATTCCTTAA ATGGTCCATT AAGCAAATAA CACCACAGCA |
| 3201 | GCAGGAGAAG AGTCCAGTAA ACACCAAATC GCTTTTCAAG |
| 3241 | CGACTTTATA GCCTTGCGCT TCACCCCAAT GCTTTCAAGA |
| 3281 | GGCTGGGAGC ATCACTTGCC TTTAATAATA TCTACAGGGA |
| 3321 | ATTCAGGGAA GAAGAGTCTC TGGTGGAACA GTTTGTGTTT |
| 3361 | GAAGCCTTGG TGATATACAT GGAGAGTCTG GCCTTAGCAC |
| 3401 | ATGCAGATGA GAAGTCCTTA GGTACAATTC AACAGTGTTG |
| 3441 | TGATGCCATT GATCACCTAT GCCGCATCAT TGAAAAGAAG |
| 3481 | CATGTTTCTT TAAATAAAGC AAAGAAACGA CGTTTGCCGC |
| 3521 | GAGGATTTCC ACCTTCCGCA TCATTGTGTT TATTGGATCT |
| 3561 | GGTCAAGTGG CTTTTAGCTC ATTGTGGGAG GCCCCAGACA |
| 3601 | GAATGTCGAC ACAAATCCAT TGAACTCTTT TATAAATTCG |
| 3641 | TTCCTTTATT GCCAGGCAAC AGATCCCCTA ATTTGTGGCT |
| 3681 | GAAAGATGTT CTCAAGGAAG AAGGTGTCTC TTTTCTCATC |
| 3721 | AACACCTTTG AGGGGGGTGG CTGTGGCCAG CCCTCGGGCA |
| 3761 | TCCTGGCCCA GCCCACCCTC TTGTACCTTC GGGGGCCATT |
| 3801 | CAGCCTGCAG GCCACGCTAT GCTGGCTGGA CCTGCTCCTG |
| 3841 | GCCGCGTTGG AGTGCTACAA CACGTTCATT GGCGAGAGAA |
| 3881 | CTGTAGGAGC GCTCCAGGTC CTAGGTACTG AAGCCCAGTC |
| 3921 | TTCACTTTTG AAAGCAGTGG CTTTCTTCTT AGAAAGCATT |
| 3961 | GCCATGCATG ACATTATAGC AGCAGAAAAG TGCTTTGGCA |
| 4001 | CTGGGGCAGC AGGTAACAGA ACAAGCCCAC AAGAGGGAGA |
| 4041 | AAGGTACAAC TACAGCAAAT GCACCGTTGT GGTCCGGATT |
| 4081 | ATGGAGTTTA CCACGACTCT GCTAAACACC TCCCCGGAAG |
| 4121 | GATGGAAGCT CCTGAAGAAG GACTTGTGTA ATACACACCT |
| 4161 | GATGAGAGTC CTGGTGCAGA CGCTGTGTGA GCCCGCAAGC |
| 4201 | ATAGGTTTCA ACATCGGAGA CGTCCAGGTT ATGGCTCATC |
| 4241 | TTCCTGATGT TTGTGTGAAT CTGATGAAAG CTCTAAAGAT |
| 4281 | GTCCCCATAC AAAGATATCC TAGAGACCCA TCTGAGAGAG |
| 4321 | AAAATAACAG CACAGAGCAT TGAGGAGCTT TGTGCCGTCA |
| 4361 | ACTTGTATGG CCCTGACGCG CAAGTGGACA GGAGCAGGCT |
| 4401 | GGCTGCTGTT GTGTCTGCCT GTAAACAGCT TCACAGAGCT |
| 4441 | GGGCTTCTGC ATAATATATT ACCGTCTCAG TCCACAGATT |
| 4481 | TGCATCATTC TGTTGGCACA GAACTTCTTT CCCTGGTTTA |
| 4521 | TAAAGGCATT GCCCCTGGAG ATGAGAGACA GTGTCTGCCT |
| 4561 | TCTCTAGACC TCAGTTGTAA GCAGCTGGCC AGCGGACTTC |
| 4501 | TGGAGTTAGC CTTTGCTTTT GGAGGACTGT GTGAGCGCCT |
| 4541 | TGTGAGTCTT CTCCTGAACC CAGCGGTGCT GTCCACGGCG |
| 4681 | TCCTTGGGCA GCTCACAGGG CAGCGTCATC CACTTCTCCC |
| 4721 | ATGGGGAGTA TTTCTATAGC TTGTTCTCAG AAACGATCAA |
| 4761 | CACGGAATTA TTGAAAAATC TGGATCTTGC TGTATTGGAG |
| 4801 | CTCATGCAGT CTTCAGTGGA TAATACCAAA ATGGTGAGTG |
| 4841 | CCGTTTTGAA CGGCATGTTA GACCAGAGCT TCAGGGAGCG |
| 4881 | AGCAAACCAG AAACACCAAG GACTGAAACT TGCGACTACA |
| 4921 | ATTCTGCAAC ACTGGAAGAA GTGTGATTCA TGGTGGGCCA |
| 4961 | AAGATTCCCC TCTCGAAACT AAAATGGCAG TGCTGGCCTT |
| 5001 | ACTGGCAAAA ATTTTACAGA TTGATTCATC TGTATCTTTT |
| 5041 | AATACAAGTC ATGGTTCATT CCCTGAAGTC TTTACAACAT |
| 5081 | ATATTAGTCT ACTTGCTGAC ACAAAGCTGG ATCTACATTT |
| 5121 | AAAGGGCCAA GCTGTCACTC TTCTTCCATT CTTCACCAGC |
| 5161 | CTCACTGGAG GCAGTCTGGA GGAACTTAGA CGTGTTCTGG |
| 5201 | AGCAGCTCAT CGTTGCTCAC TTCCCCATGC AGTCCAGGGA |
| 5241 | ATTTCCTCCA GGAACTCCGC GGTTCAATAA TTATGTGGAC |
| 5281 | TGCATGAAAA AGTTTCTAGA TGCATTGGAA TTATCTCAAA |
| 5321 | GCCCTATGTT GTTGGAATTG ATGACAGAAG TTCTTTGTCG |
| 5361 | GGAACAGCAG CATGTCATGG AAGAATTATT TCAATCCAGT |
| 5401 | TTCAGGAGGA TTGCCAGAAG GGGTTCATGT GTCACACAAG |
| 5441 | TAGGCCTTCT GGAAAGCGTG TATGAAATGT TCAGGAAGGA |
| 5481 | TGACCCCCGC CTAAGTTTCA CACGCCAGTC CTTTGTGGAC |
| 5521 | CGCTCCCTCC TCACTCTGCT GTGGCACTGT AGCCTGGATG |
| 5561 | CTTTGAGAGA ATTCTTCAGC ACAATTGTGG TGGATGCCAT |
| 5601 | TGATGTGTTG AAGTCCAGGT TTACAAAGCT AAATGAATCT |
| 5641 | ACCTTTGATA CTCAAATCAC CAAGAAGATG GGCTACTATA |
| 5681 | AGATTCTAGA CGTGATGTAT TCTCGCCTTC CCAAAGATGA |
| 5721 | TGTTCATGCT AAGGAATCAA AAATTAATCA AGTTTTCCAT |
| 5761 | GGCTCGTGTA TTACAGAAGG AAATGAACTT ACAAAGACAT |
| 5801 | TGATTAAATT GTGCTACGAT GCATTTACAG AGAACATGGC |
| 5841 | AGGAGAGAAT CAGCTGCTGG AGAGGAGAAG ACTTTACCAT |
| 5881 | TGTGCAGCAT ACAACTGCGC CATATCTGTC ATCTGCTGTG |
| 5921 | TCTTCAATGA GTTAAAATTT TACCAAGGTT TTCTGTTTAG |
| 5961 | TGAAAAACCA GAAAAGAACT TGCTTATTTT TGAAAATCTG |
| 6001 | ATCGACCTGA AGCGCCGCTA TAATTTTCCT GTAGAAGTTG |
| 6041 | AGGTTCCTAT GGAAAGAAAG AAAAAGTACA TTGAAATTAG |
| 6081 | GAAAGAAGCC AGAGAAGCAG CAAATGGGGA TTCAGATGGT |
| 6121 | CCTTCCTATA TGTCTTCCCT GTCATATTTG GCAGACAGTA |
| 6161 | CCCTGAGTGA GGAAATGAGT CAATTTGATT TCTCAACCGG |
| 6201 | AGTTCAGAGC TATTCATACA GCTCCCAAGA CCCTAGACCT |
| 6241 | GCCACTGGTC GTTTTCGGAG ACGGGAGCAG CGGGACCCCA |
| 6281 | CGGTGCATGA TGATGTGCTG GAGCTGGAGA TGGACGAGCT |
| 6321 | CAATCGGCAT GAGTGCATGG CGCCCCTGAC GGCCCTGGTC |
| 6361 | AAGCACATGC ACAGAAGCCT GGGCCCGCCT CAAGGAGAAG |
| 6401 | AGGATTCAGT GCCAAGAGAT CTTCCTTCTT GGATGAAATT |
| 6441 | CCTCCATGGC AAACTGGGAA ATCCAATAGT ACCATTAAAT |
| 6481 | ATCCGTCTCT TCTTAGCCAA GCTTGTTATT AATACAGAAG |
| 6521 | AGGTCTTTCG CCCTTACGCG AAGCACTGGC TTAGCCCCTT |
| 6561 | GCTGCAGCTG GCTGCTTCTG AAAACAATGG AGGAGAAGGA |
| 6601 | ATTCACTACA TGGTGGTTGA GATAGTGGCC ACTATTCTTT |
| 6641 | CATGGACAGG CTTGGCCACT CCAACAGGGG TCCCTAAAGA |
| 6681 | TGAAGTGTTA GCAAATCGAT TGCTTAATTT CCTAATGAAA |
| 6721 | CATGTCTTTC ATCCAAAAAG AGCTGTGTTT AGACACAACC |
| 6761 | TTGAAATTAT AAAGACCCTT GTCGAGTGCT GGAAGGATTG |
| 6801 | TTTATCCATC CCTTATAGGT TAATATTTGA AAAGTTTTCC |
| 6841 | GGTAAAGATC CTAATTCTAA AGACAACTCA GTAGGGATTC |
| 6881 | AATTGCTAGG CATCGTGATG GCCAATGACC TGCCTCCCTA |
| 6921 | TGACCCACAG TGTGGCATCC AGAGTAGCGA ATACTTCCAG |
| 6961 | GCTTTGGTGA ATAATATGTC CTTTGTAAGA TATAAAGAAG |
| 7001 | TGTATGCCGC TGCAGCAGAA GTTCTAGGAC TTATACTTCG |
| 7041 | ATATGTTATG GAGAGAAAAA ACATACTGGA GGAGTCTCTG |
| 7081 | TGTGAACTGG TTGCGAAACA ATTGAAGCAA CATCAGAATA |
| 7121 | CTATGGAGGA CAAGTTTATT GTGTGCTTGA ACAAAGTGAC |
| 7161 | CAAGAGCTTC CCTCCTCTTG CAGACAGGTT CATGAATGCT |
| 7201 | GTGTTCTTTC TGCTGCCAAA ATTTCATGGA GTGTTGAAAA |
| 7241 | CACTCTGTCT GGAGGTGGTA CTTTGTCGTG TGGAGGGAAT |
| 7281 | GACAGAGCTG TACTTCCAGT TAAAGAGCAA GGACTTCGTT |
| 7321 | CAAGTCATGA GACATAGAGA TGATGAAAGA CAAAAAGTAT |
| 7361 | GTTTGGACAT AATTTATAAG ATGATGCCAA AGTTAAAACC |
| 7401 | AGTAGAACTC CGAGAACTTC TGAACCCCGT TGTGGAATTC |
| 7441 | GTTTCCCATC CTTCTACAAC ATGTAGGGAA CAAATGTATA |
| 7481 | ATATTCTCAT GTGGATTCAT GATAATTACA GAGATCCAGA |
| 7521 | AAGTGAGACA GATAATGACT CCCAGGAAAT ATTTAAGTTG |
| 7561 | GCAAAAGATG TGCTGATTCA AGGATTGATC GATGAGAACC |
| 7601 | CTGGACTTCA ATTAATTATT CGAAATTTCT GGAGCCATGA |
| 7641 | AACTAGGTTA CCTTCAAATA CCTTGGACCG GTTGCTGGCA |
| 7681 | CTAAATTCCT TATATTCTCC TAAGATAGAA GTGCACTTTT |
| 7721 | TAAGTTTAGC AACAAATTTT CTGCTCGAAA TGACCAGCAT |
| 7761 | GAGCCCAGAT TATCCAAACC CCATGTTCGA GCATCCTCTG |
| 7801 | TCAGAATGCG AATTTCAGGA ATATACCATT GATTCTGATT |
| 7841 | GGCGTTTCCG AAGTACTGTT CTCACTCCGA TGTTTGTGGA |
| 7881 | GACCCAGGCC TCCCAGGGCA CTCTCCAGAC CCGTACCCAG |
| 7921 | GAAGGGTCCC TCTCAGCTCG CTGGCCAGTG GCAGGGCAGA |
| 7961 | TAAGGGCCAC CCAGCAGCAG CATGACTTCA CACTGACACA |
| 8001 | GACTGCAGAT GGAAGAAGCT CATTTGATTG GCTGACCGGG |
| 8041 | AGCAGCACTG ACCCGCTGGT CGACCACACC AGTCCCTCAT |
| 8081 | CTGACTCCTT GCTGTTTGCC CACAAGAGGA GTGAAAGGTT |
| 8121 | ACAGAGAGCA CCCTTGAAGT CAGTGGGGCC TGATTTTGGG |
| 8161 | AAAAAAAGGC TGGGCCTTCC AGGGGACGAG GTGGATAACA |
| 8201 | AAGTGAAAGG TGCGGCCGGC CGGACGGACC TACTACGACT |
| 8241 | GCGCAGACGG TTTATGAGGG ACCAGGAGAA GCTCAGTTTG |
| 8281 | ATGTATGCCA GAAAAGGCGT TGCTGAGCAA AAACGAGAGA |
| 8321 | AGGAAATCAA GAGTGAGTTA AAAATGAAGC AGGATGCCCA |
| 8361 | GGTCGTTCTG TACAGAAGCT ACCGGCACGG AGACCTTCCT |
| 8401 | GACATTCAGA TCAAGCACAG CAGCCTCATC ACCCCGTTAC |
| 8441 | AGGCCGTGGC CCAGAGGGAC CCAATAATTG CAAAACAGCT |
| 8481 | CTTTAGCAGC TTGTTTTCTG GAATTTTGAA AGAGATGGAT |
| 8521 | AAATTTAAGA CACTGTCTGA AAAAAACAAC ATCACTCAAA |
| 8561 | AGTTGCTTCA AGACTTCAAT CGTTTTCTTA ATACCACCTT |
| 8601 | CTCTTTCTTT CCACCCTTTG TCTCTTGTAT TCAGGACATT |
| 8641 | AGCTGTCAGC ACGCAGCCCT GCTGAGCCTC GACCCAGCGG |
| 8681 | CTGTTAGCGC TGGTTGCCTG GCCAGCCTAC AGCAGCCCGT |
| 8721 | GGGCATCCGC CTGCTAGAGG AGGCTCTGCT CCGCCTGCTG |
| 8761 | CCTGCTGAGC TGCCTGCCAA GCGAGTCCGT GGGAAGGCCC |
| 8801 | GCCTCCCTCC TGATGTCCTC AGATGGGTGG AGCTTGCTAA |
| 8841 | GCTGTATAGA TCAATTGGAG AATACGACGT CCTCCGTGGG |
| 8881 | ATTTTTACCA GTGAGATAGG AACAAAGCAA ATCACTCAGA |
| 8921 | GTGCATTATT AGCAGAAGCC AGAAGTGATT ATTCTGAAGC |
| 8961 | TGCTAAGCAG TATGATGAGG CTCTCAATAA ACAAGACTGG |
| 9001 | GTAGATGGTG AGCCCACAGA AGCCGAGAAG GATTTTTGGG |
| 9041 | AACTTGCATC CCTTGACTGT TACAACCACC TTGCTGAGTG |
| 9081 | GAAATCACTT GAATACTGTT CTACAGCCAG TATAGACAGT |
| 9121 | GAGAACCCCC CAGACCTAAA TAAAATCTGG AGTGAACCAT |
| 9161 | TTTATCAGGA AACATATCTA CCTTACATGA TCCGCAGCAA |
| 9201 | GCTGAAGCTG CTGCTCCAGG GAGAGGCTGA CCAGTCCCTG |
| 9241 | CTGACATTTA TTGACAAAGC TATGCACGGG GAGCTCCAGA |
| 9281 | AGGCGATTCT AGAGCTTCAT TACAGTCAAG AGCTGAGTCT |
| 9321 | GCTTTACCTC CTGCAAGATG ATGTTGACAG AGCCAAATAT |
| 9361 | TACATTCAAA ATGGCATTCA GAGTTTTATG CAGAATTATT |
| 9401 | CTAGTATTGA TGTCCTCTTA CACCAAAGTA GACTCACCAA |
| 9441 | ATTGCAGTCT GTACAGGCTT TAACAGAAAT TCAGGAGTTC |
| 9481 | ATCAGCTTTA TAAGCAAACA AGGCAATTTA TCATCTCAAG |
| 9521 | TTCCCCTTAA GAGACTTCTG AACACCTGGA CAAACAGATA |
| 9561 | TCCAGATGCT AAAATGGACC CAATGAACAT CTGGGATGAC |
| 9601 | ATCATCACAA ATCGATGTTT CTTTCTCAGC AAAATAGAGG |
| 9641 | AGAAGCTTAC CCCTCTTCCA GAAGATAATA GTATGAATGT |
| 9681 | GGATCAAGAT GGAGACCCCA GTGACAGGAT GGAAGTGCAA |
| 9721 | GAGCAGGAAG AAGATATCAG CTCCCTGATC AGGAGTTGCA |
| 9761 | AGTTTTCCAT GAAAATGAAG ATGATAGACA GTGCCCGGAA |
| 9801 | GCAGAACAAT TTCTCACTTG CTATGAAACT ACTGAAGGAG |
| 9841 | CTGCATAAAG AGTCAAAAAC CAGAGACGAT TGGCTGGTGA |
| 9881 | GCTGGGTGCA GAGCTACTGC CGCCTGAGCC ACTGCCGGAG |
| 9921 | CCGGTCCCAG GGCTGCTCTG AGCAGGTGCT CACTGTGCTG |
| 9961 | AAAACAGTCT CTTTGTTGGA TGAGAACAAC GTGTCAAGCT |
| 10001 | ACTTAAGCAA AAATATTCTG GCTTTCCGTG ACCAGAACAT |
| 10041 | TCTCTTGGGT ACAACTTACA GGATCATAGC GAATGCTCTC |
| 10081 | AGCAGTGAGC CAGCCTGCCT TGCTGAAATC GAGGAGGACA |
| 10121 | AGGCTAGAAG AATCTTAGAG CTTTCTGGAT CCAGTTCAGA |
| 10161 | GGATTCAGAG AAGGTGATCG CGGGTCTGTA CCAGAGAGCA |
| 10201 | TTCCAGCACC TCTCTGAGGC TGTGCAGGCG GCTGAGGAGG |
| 10241 | AGGCCCAGCC TCCCTCCTGG AGCTGTGGGC CTGCAGCTGG |
| 10281 | GGTGATTGAT GCTTACATGA CGCTGGCAGA TTTCTGTGAC |
| 10321 | CAACAGCTGC GCAAGGAGGA AGAGAATGCA TCAGTTATTG |
| 10361 | ATTCTGCAGA ACTGCAGGCG TATCCAGCAC TTGTGGTGGA |
| 10401 | GAAAATGTTG AAAGCTTTAA AATTAAATTC CAATGAAGCC |
| 10441 | AGATTGAAGT TTCCTAGATT ACTTCAGATT ATAGAACGGT |
| 10481 | ATCCAGAGGA GACTTTGAGC CTCATGACAA AAGAGATCTC |
| 10521 | TTCCGTTCCC TGCTGGCAGT TCATCAGCTG GATCAGCCAC |
| 10561 | ATGGTGGCCT TACTGGACAA AGACCAAGCC GTTGCTGTTC |
| 10601 | AGCACTCTGT GGAAGAAATC ACTGATAACT ACCCGCAGGC |
| 10641 | TATTGTTTAT CCCTTCATCA TAAGCAGCGA AAGCTATTCC |
| 10681 | TTCAAGGATA CTTCTACTGG TCATAAGAAT AAGGAGTTTG |
| 10721 | TGGCAAGGAT TAAAAGTAAG TTGGATCAAG GAGGAGTGAT |
| 10761 | TCAAGATTTT ATTAATGCCT TAGATCAGCT CTCTAATCCT |
| 10801 | GAACTGCTCT TTAAGGATTG GAGCAATGAT GTAAGAGCTG |
| 10901 | AACTAGCAAA AACCCCTGTA AATAAAAAAA ACATTGAAAA |
| 10941 | AATGTATGAA AGAATGTATG CAGCCTTGGG TGACCCAAAG |
| 10921 | GCTCCAGGCC TGGGGGCCTT TAGAAGGAAG TTTATTCAGA |
| 10961 | CTTTTGGAAA AGAATTTGAT AAACATTTTG GGAAAGGAGG |
| 11001 | TTCTAAACTA CTGAGAATGA AGCTCAGTGA CTTCAACGAC |
| 11041 | ATTACCAACA TGCTACTTTT AAAAATGAAC AAAGACTCAA |
| 11081 | AGCCCCCTGG GAATCTGAAA GAATGTTCAC CCTGGATGAG |
| 11121 | CGACTTCAAA GTGGAGTTCC TGAGAAATGA GCTGGAGATT |
| 11161 | CCCGGTCAGT ATGACGGTAG GGGAAAGCCA TTGCCAGAGT |
| 11201 | ACCACGTGCG AATCGCCGGG TTTGATGAGC GGGTGACAGT |
| 11241 | CATGGCGTCT CTGCGAAGGC CCAAGCGCAT CATCATCCGT |
| 11281 | GGCCATGACG AGAGGGAACA CCCTTTCCTG GTGAAGGGTG |
| 11321 | GCGAGGACCT GCGGCAGGAC CAGCGCGTGG AGCAGCTCTT |
| 11361 | CCAGGTCATG AATGGGATCC TGGCCCAAGA CTCCGCCTGC |
| 11401 | AGCCAGAGGG CCCTGCAGCT GAGGACCTAT AGCGTTGTGC |
| 11441 | CCATGACCTC CAGGTTAGGA TTAATTGAGT GGCTTGAAAA |
| 11481 | TACTGTTACC TTGAAGGACC TTCTTTTGAA CACCATGTCC |
| 11521 | CAAGAGGAGA AGGCGGCTTA CCTGAGTGAT CCCAGGGCAC |
| 11561 | CGCCGTGTGA ATATAAAGAT TGGCTGACAA AAATGTCAGG |
| 11601 | AAAACATGAT GTTGGAGCTT ACATGCTAAT GTATAAGGGC |
| 11641 | GCTAATCGTA CTGAAACAGT CACGTCTTTT AGAAAACGAG |
| 11681 | AAAGTAAAGT GCCTGCTGAT CTCTTAAAGC GGGCCTTCGT |
| 11721 | GAGGATGAGT ACAAGCCCTG AGGCTTTCCT GGCGCTCCGC |
| 11761 | TCCCACTTCG CCAGCTCTCA CGCTCTGATA TGCATCAGCC |
| 11801 | ACTGGATCCT CGGGATTGGA GACAGACATC TGAACAACTT |
| 11841 | TATGGTGGCC ATGGAGACTG GCGGCGTGAT CGGGATCGAC |
| 11881 | TTTGGGCATG CGTTTGGATC CGCTACACAG TTTCTGCCAG |
| 11921 | TCCCTGAGTT GATGCCTTTT CGGCTAACTC GCCAGTTTAT |
| 11961 | CAATCTGATG TTACCAATGA AAGAAACGGG CCTTATGTAC |
| 12001 | AGCATCATGG TACACGCACT CCGGGCCTTC CGCTCAGACC |
| 12041 | CTGGCCTGCT CACCAACACC ATGGATGTGT TTGTCAAGGA |
| 12081 | GCCCTCCTTT GATTGGAAAA ATTTTGAACA GAAAATGCTG |
| 12121 | AAAAAAGGAG GGTCATGGAT TCAAGAAATA AATGTTGCTG |
| 12161 | AAAAAAATTG GTACCCCCGA CAGAAAATAT GTTACGCTAA |
| 12201 | GAGAAAGTTA GCAGGTGCCA ATCCAGCAGT CATTACTTGT |
| 12241 | GATGAGCTAC TCCTGGGTCA TGAGAAGGCC CCTGCCTTCA |
| 12281 | GAGACTATGT GGCTGTGGCA CGAGGAAGCA AAGATCACAA |
| 12321 | CATTCGTGCC CAAGAACCAG AGAGTGGGCT TTCAGAAGAG |
| 12361 | ACTCAAGTGA AGTGCCTGAT GGACCAGGCA ACAGACCCCA |
| 12401 | ACATCCTTGG CAGAACCTGG GAAGGATGGG AGCCCTGGAT |
| 12441 | GTGAGGTCTG TGGGAGTCTG CAGATAGAAA GCATTACATT |
| 12481 | GTTTAAAGAA TCTACTATAC TTTGGTTGGC AGCATTCCAT |
| 12521 | GAGCTGATTT TCCTGAAACA CTAAAGAGAA ATGTCTTTTG |
| 12561 | TGCTACAGTT TCGTAGCATG AGTTTAAATC AAGATTATGA |
| 12601 | TGAGTAAATG TGTATGGGTT AAATCAAAGA TAAGGTTATA |
| 12641 | GTAACATCAA AGATTAGGTG AGGTTTATAG AAAGATAGAT |
| 12681 | ATCCAGGCTT ACCAAAGTAT TAAGTCAAGA ATATAATATG |
| 12721 | TGATCAGCTT TCAAAGCATT TACAAGTGCT GCAAGTTAGT |
| 12761 | GAAACAGCTG TCTCCGTAAA TGGAGGAAAT GTGGGGAAGC |
| 12801 | CTTGGAATGC CCTTCTGGTT CTGGCACATT GGAAAGCACA |
| 12841 | CTCAGAAGGC TTCATCACCA AGATTTTGGG AGAGTAAAGC |
| 12881 | TAAGTATAGT TGATGTAACA TTGTAGAAGC AGCATAGGAA |
| 12921 | CAATAAGAAC AATAGGTAAA GCTATAATTA TGGCTTATAT |
| 12961 | TTAGAAATGA CTGCATTTGA TATTTTAGGA TATTTTTCTA |
| 13001 | GGTTTTTTCC TTTCATTTTA TTCTCTTCTA GTTTTGACAT |
| 13041 | TTTATGATAG ATTTGCTCTC TAGAAGGAAA CGTCTTTATT |
| 13081 | TAGGAGGGCA AAAATTTTGG TCATAGCATT CACTTTTGCT |
| 13121 | ATTCCAATCT ACAACTGGAA GATACATAAA AGTGCTTTGC |
| 13161 | ATTGAATTTG GGATAACTTC AAAAATCCCA TGGTTGTTGT |
| 13201 | TAGGGATAGT ACTAAGCATT TCAGTTCCAG GAGAATAAAA |
| 13241 | GAAATTCCTA TTTGAAATGA ATTCCTCATT TGGAGGAAAA |
| 13281 | AAAGCATGCA TTCTAGCACA ACAAGATGAA ATTATGGAAT |
| 13321 | ACAAAAGTGG CTCCTTCCCA TGTGCAGTCC CTGTCCCCCC |
| 13361 | CCGCCAGTCC TCCACACCCA AACTGTTTCT GATTGGCTTT |
| 13401 | TAGCTTTTTG TTGTTTTTTT TTTTCCTTCT AACACTTGTA |
| 13441 | TTTGGAGGCT CTTCTGTGAT TTTGAGAAGT ATACTCTTGA |
| 13481 | GTGTTTAATA AAGTTTTTTT CCAAAAGTA |

Another example of a DNA-PKcs polypeptide amino acid sequence is found in the NCBI database at accession number AAC52019 (gi: 9188646), and is reproduced below (SEQ ID NO:3).

| | |
|---|---|
| 1 | MAGSGAGVRC SLLRLQETLS AADRCGAALA GHQLIRGLGQ |
| 41 | ECVLSSSPAV LALQTSLVFS RDFGLLVFVR KSLNSIEFRE |
| 81 | CREEILKFLC IFLEKMGQKI APYSVEIKNT CTSVYTKDRA |
| 121 | AKCKIPALDL LIKLLQTFRS SRLMDEFKIG ELFSKFYGEL |
| 161 | ALKKKIPDTV LEKVYELLGL LGEVHPSEMI NNAENLFRAF |
| 201 | LGELKTQMTS AVREPKLPVL AGCLKGLSSL LCNFTKSMEE |
| 241 | DPQTSREIFN FVLKAIRPQI DLKRYAVPSA GLRLFALHAS |
| 281 | QFSTCLLDNY VSLFEVLLKW CAHTNVELKK AALSALESFL |
| 321 | KQVSNMVAKN AEMHKNKLQY FMEQFYGIIR NVDSNNKELS |
| 361 | IAIRGYGLFA GPCKVINAKD VDFMYVELIQ RCKQMFLTQT |
| 401 | DTGDDRVYQM PSFLQSVASV LLYLDTVPEV YTPVLEHLVV |
| 441 | MQIDSFPQYS PKMQLVCCRA IVKVFLALAA KGPVLRNCIS |
| 481 | TVVHQGLIRI CSKPVVLPKG PESESEDHRA SGEVRTGKWK |
| 521 | VPTYKDYVDL FRHLLSSDQM MDSILADEAF FSVNSSSESL |
| 561 | NHLLYDEFVK SVLKIVEKLD LTLEIQTVGE QENGDEAPGV |
| 601 | WMIPTSDPAA NLHPAKPKDF SAFINLVEFC REILPEKQAE |
| 641 | FFEPWVYSFS YELILQSTRL PLISGFYKLL SITVRNAKKI |
| 681 | KYFEGVSPKS LKHSPEDPEK YSCFALFVKF GKEVAVKMKQ |
| 721 | YKDELLASCL TFLLSLPHNI IELDVRAYVP ALQMAFKLGL |
| 761 | SYTPLAEVGL NALEEWSIYI DRHVMQPYYK DILPCLDGYL |
| 801 | KTSALSDETK NNWEVSALSR AAQKGFNKVV LKHLKKTKNL |
| 841 | SSNEAISLEE IRIRVVQMLG SLGGQINKNL LTVTSSDEMM |
| 881 | KSYVAWDREK RLSFAVPFRE MKPVIFLDVF LPRVTELALT |
| 921 | ASDRQTKVAA CELLHSMVMF MLGKATQMPE GGQGAPPMYQ |
| 961 | LYKRTFPVLL RLACDVDQVT RQLYEPLVMQ LIHWFTNNKK |
| 1001 | FESQDTVALL EAILDGIVDP VDSTLRDFCG RCIREFLKWS |
| 1041 | IKQITPQQQE KSPVNTKSLF KRLYSLALHP NAFKRLGASL |
| 1081 | AFNNIYREFR EEESLVEQFV FEALVIYMES LALAHADEKS |
| 1121 | LGTIQQCCDA IDHLCRIIEK KHVSLNKAKK RRLPRGFPPS |
| 1161 | ASLCLLDLVK WLLAHCGRPQ TECRHKSIEL FYKFVPLLPG |
| 1201 | NRSPNLWLKD VLKEEGVSFL INTFEGGGCG QPSGILAQPT |
| 1241 | LLYLRGPFSL QATLCWLDLL LAALECYNTF IGERTVGALQ |
| 1281 | VLGTEAQSSL LKAVAFFLES IAMHDIIAAE KCFGTGAAGN |
| 1321 | RTSPQEGERY NYSKCTVVVR IMEFTTTLLN TSPEGWKLLK |
| 1361 | KDLCNTHLMR VLVQTLCEPA SIGFNIGDVQ VMAHLPDVCV |
| 1401 | NLMKALKMSP YKDILETHLR EKITAQSIEE LCAVNLYGPD |
| 1441 | AQVDRSRLAA VVSACKQLHR AGLLHNILPS QSTDLHHSVG |
| 1481 | TELLSLVYKG IAPGDERQCL PSLDLSCKQL ASGLLELAFA |
| 1521 | FGGLCERLVS LLLNPAVLST ASLGSSQGSV IHFSHGEYFY |
| 1561 | SLFSETINTE LLKNLDLAVL ELMQSSVDNT KMVSAVLNGM |
| 1601 | LDQSFRERAN QKHQGLKLAT TILQHWKKCD SWWAKDSPLE |
| 1641 | TKMAVLALLA KILQIDSSVS FNTSHGSFPE VFTTYISLLA |
| 1681 | DTKLDLHLK |

A nucleotide sequence for this DNA-PKcs polypeptide is provided by the NCBI database as accession number U63630 (gi: 18497329).

As described herein, DNA-PKcs has a much larger role during aging than simply DNA repair. In particular, as demonstrated herein DNA-PKcs influences aging, glucose responses, weight management, energy levels, brain function (memory, object recognition, anxiety, stress, depression), physical fitness (stamina, endurance, mitochondrial function) and the like. In particular, as demonstrated herein, DNA-PKcs expression or activity is correlated with a greater tendency towards obesity, high blood pressure, lower numbers of mitochondria, diminished stamina during physical activity, insulin insensitivity, higher blood glucose levels, increased anxiety, poor memory and/or object recognition, depression and the like.

Thus, according to the invention, when DNA-PKcs expression and/or activity is inhibited in mammals, those mammals exhibit less weight gain, higher numbers of mitochondria, greater stamina, lower blood pressure, increased thermogenesis, insulin sensitivity, improved insulin signaling, improved memory, improved learning, reduced depression, reduced anxiety and the like. These effects are surprising in view of currently available information, which indicates that loss of DNA-PKcs in a mammal can increase the incidence of tumors.

Accordingly, the present invention involves methods of controlling weight gain, increasing mitochondria, improving stamina, reducing blood pressure, increasing thermogenesis, improving insulin sensitivity, improving insulin signaling, improving memory, improving learning, reducing depression, reducing anxiety and the like in a mammal, by administering to the mammal an effective amount of a DNA-PKcs inhibitor.

SCID (Severe Combined Immune Deficiency) mice, which carry a leaky nonsense mutation that truncates 83 amino acids from the C-terminus end of the kinase domain of DNA-PKcs, have a block in lymphocyte development. Although DNA-PKcs is expressed ubiquitously, DNA-PKcs-deficient mice develop normally and DNA-PKcs-deficient fibroblasts grow well in culture. Fibroblasts deficient in other DNA repair proteins often grow very poorly. This may be explained by the observation that the importance of DNA-PKcs in DNA repair depends on the level of DNA damage: at low levels, other DNA repair proteins dominate the repair process. Thus, mammals with diminished expression of DNA-PKcs or a defective DNA-PKcs gene generally have improved health relative to those with high levels of DNA-PKcs gene expression, particularly when the mammals are older mammals. Moreover, mice with diminished expression of DNA-PKcs or a defective DNA-PKcs gene, in the whole body or in specific tissues, are useful animal models for testing the effects of DNA-PKcs inhibition and developing appropriate therapeutic dosages and regiments for administration of DNA-PKcs inhibitors.

### DNA-PK Inhibitors

Any available method of inhibiting DNA-PKcs or inhibitor of DNA-PKcs can be used in the compositions and methods of the invention. For example, DNA-PKcs deficiency, DNA-PKcs suppression by DNA-PKcs inhibitors/antagonists, or DNA-PKcs knock-down with DNA-PKcs siRNA can be used for inhibiting DNA-PKcs expression or activity. While the term DNA-PK refers to a larger complex and DNA-PKcs refers to the catalytic subunit of DNA-PK, the terms “DNA-PK inhibitor” and “DNA-PKcs inhibitor” are used interchangeably and have the same meaning—a compound or agent that can reduce the activity of the DNA-PK complex and/or the DNA-PK catalytic subunit.

Any compound that can inhibit DNA-PK can be used in the methods and compositions of the invention. For example, the DNA-PK inhibitor can be a compound of formula I:

R₁—Ar—R₂(R₃)ₙ I

wherein:

R₁ is a hydrogen, lower alkoxy, cycloaryl, cycloheteroaryl, cycloalkyl or cycloheteroalkyl, wherein the cycloaryl, cycloheteroaryl, cycloalkyl and cycloheteroalkyl can optionally be substituted with one to four substituents selected from the group consisting of halo, hydroxy, lower alkyl, lower alkoxy, cyano, aryl, and heteroaryl;

Ar is cycloaryl or cycloheteroaryl that can be substituted with one or two oxy (═O) or thio (═S or —SH) groups;

R₂ is cycloheteroaryl or cycloheteroalkyl;

R₃ is halo, lower alkyl, lower alkoxy, cyano, aryl, and heteroaryl; and

n is an integer of 0-3.

In some embodiments, R₁ is hydrogen. Examples of other R₁ substituents that can be used in the compounds, compositions and methods of the invention include the following:

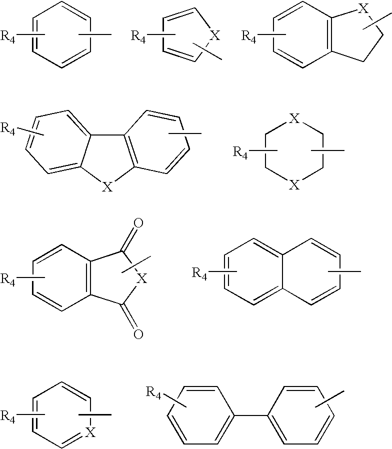

wherein X is a heteroatom, R₄ is hydrogen, halo, hydroxy, lower alkyl, lower alkoxy, cyano, aryl, and heteroaryl.

Ar can include a variety of substituents such as phenyl, indenyl, naphthyl, furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide). In some embodiments, Ar is one of the following:

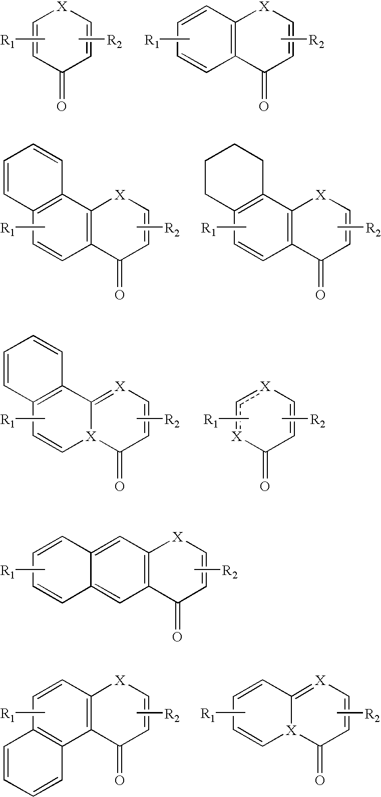

wherein X is a heteroatom and the R₁ and R₂ groups are as defined herein.

As stated above, R₂ is cycloheteroaryl or cycloheteroalkyl. Examples of R₂ cycloheteroaryl and cycloheteroalkyl substituents include

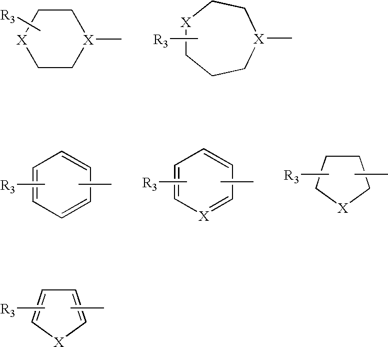

wherein R₃ is halo, lower alkyl, lower alkoxy, cyano, aryl, and heteroaryl.

In other embodiments, the DNA-PKcs inhibitor can be a compound of formula II:

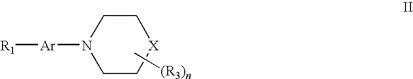

wherein R₁, Ar, R₃ and n are as defined above, and X is a heteroatom. In some embodiments X selected from the group consisting of O, NH or S. In other embodiments, X is oxygen.

The following definitions are used, unless otherwise described: halo is fluoro, chloro, bromo, or iodo. Alkyl, alkoxy, alkenyl, alkynyl, etc. denote both straight and branched groups. Aryl denotes a phenyl radical or an ortho-fused bicyclic or tricyclic carbocyclic radical having about nine to fourteen ring atoms in which at least one ring is aromatic. Heteroatom is a non-peroxide oxygen, sulfur, or N(R₆) wherein R₆ is absent or is H, O, (C₁-C₄)alkyl. Cycloheteroaryl encompasses a radical attached via a ring carbon of a cyclic aromatic ring containing five or six ring atoms consisting of carbon and one to four heteroatoms each selected from the group consisting of non-peroxide oxygen, sulfur, and N(R₆) wherein R₆ is absent or is H, O, (C₁-C₄)alkyl, phenyl or benzyl, as well as a radical of an ortho-fused bicyclic or tricyclic heterocycle of about eight to fifeen ring atoms derived therefrom.

Specific and preferred values listed below for radicals, substituents, and ranges, are for illustration only; they do not exclude other defined values or other values within defined ranges for the radicals and substituents.

Specifically, lower alkyl is (C₁-C₆)alkyl and can be methyl, ethyl, propyl, isopropyl, butyl, iso-butyl, sec-butyl, pentyl, 3-pentyl, or hexyl; cycloalkyl is (C₃-C₆)cycloalkyl or (C₃-C₆)cycloalkyl(C₁-C₆)alkyl, where (C₃-C₆)cycloalkyl is cyclopropyl, cyclobutyl, cyclopentyl, or cyclohexyl and (C₃-C₆)cycloalkyl(C₁-C₆)alkyl is cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; lower alkoxy is (C₁-C₆)alkoxy which can be methoxy, ethoxy, propoxy, isopropoxy, butoxy, iso-butoxy, sec-butoxy, pentoxy, 3-pentoxy, or hexyloxy; lowere alkenyl is (C₂-C₆)alkenyl which can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1- hexenyl, 2-hexenyl, 3-hexenyl, 4-hexenyl, or 5-hexenyl; lower alkynyl is (C₂-C₆)alkynyl which can be ethynyl, 1-propynyl, 2-propynyl, 1-butynyl, 2-butynyl, 3-butynyl, 1-pentynyl, 2-pentynyl, 3-pentynyl, 4-pentynyl, 1- hexynyl, 2-hexynyl, 3-hexynyl, 4-hexynyl, or 5-hexynyl; lower alkanoyl is (C₁-C₆)alkanoyl which can be acetyl, propanoyl or butanoyl; lower haloalkyl is halo(C₁-C₆)alkyl which can be iodomethyl, bromomethyl, chloromethyl, fluoromethyl, trifluoromethyl, 2-chloroethyl, 2-fluoroethyl, 2,2,2-trifluoroethyl, or pentafluoroethyl; lower hydroxyalkyl is hydroxy(C₁-C₆)alkyl which can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; lower alkoxycarbonyl is (C₁-C₆)alkoxycarbonyl which can be methoxycarbonyl, ethoxycarbonyl, propoxycarbonyl, isopropoxycarbonyl, butoxycarbonyl, pentoxycarbonyl, or hexyloxycarbonyl; lower alkylthio is (C₁-C₆)alkylthio which can be methylthio, ethylthio, propylthio, isopropylthio, butylthio, isobutylthio, pentylthio, or hexylthio; lower alkanoyloxy is (C₂-C₆)alkanoyloxy which can be acetoxy, propanoyloxy, butanoyloxy, isobutanoyloxy, pentanoyloxy, or hexanoyloxy.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine DNA-PK inhibitory activity using the standard tests described herein, or using other similar tests which are well known in the art.

Examples of inhibitors of DNA-PKcs include NU7026, Euk-134, MnTBAP, 2,4-dinitrophenol (DNP), metformin, resveratrol, chromen-4-one compounds and nucleic acids that can inhibit the expression and/or translation of DNA-PKcs.

Examples of cells where DNA-PKcs can be inhibited include any cell type where DNA-PKcs may be expressed. Such cells include endodermal, mesodermal, ectodermal cells. Other examples of types of cells where DNA-PKcs expression/activity may be inhibited include adipose cells, muscle cells, endothelial cells, heart cells, liver cells, lymphocytes, intestinal cells, kidney cells, brain cells, neuronal cells and any combination thereof.

An inhibitor can reduce the expression and/or activity of DNA-PKcs by any amount. In some embodiments, residual levels of DNA-PKcs activity/expression are retained, for example, to permit DNA-PKcs to perform some DNA double-stranded break repair and V(D)J recombination. For example, DNA-PKcs can be inhibited by 2%, 5%, 10%, 20%, 40% or more than 40%. In other embodiments, DNA-PKcs activity/expression is substantially inhibited, such as, for example, by 70%, 75%, 80%, 85%, 90%, 95%, 96%, 97%, 98% or 99%.

NU7026 (2-(morpholin-4-yl)-benzo[h]chomen-4-one) is an ATP-competitive inhibitor of DNA-dependent protein kinases (DNA-PK). The structure of NU7026 is shown below.

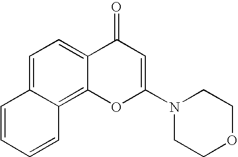

NU7026 (2-(morpholin-4-yl)-benzo[h]chomen-4-one) is a cell permeable DNA-PK inhibitor which has been shown to sensitize mouse embryonic fibroblasts and Chinese hamster ovary cells to radiation in vitro (Veuger et al, Cancer Res 63:6008, 2003; Griffin et al. J Med Chem 48:569, 2005, which is specifically incorporated herein by reference in its entirety). NU7026 was shown to sensitize leukemic cells to topoisomerase II inhibitors (Willmore E et al. Blood 103:4659, 2004, which is specifically incorporated herein by reference in its entirety).

Resveratrol is a natural polyphenolic compound found in the skin of grapes and is known for its phytoestrogenic and antioxidant properties (Baur and Sinclair Nat. Rev. Drug Discov. 5:493, 2006, which is specifically incorporated herein by reference in its entirety). The structure of resveratrol is provided below.

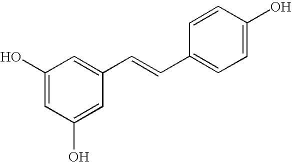

LY294002 is another DNA-PKcs inhibitor having the following structure.

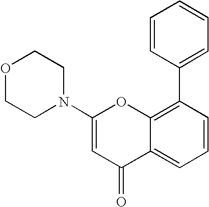

Synthetic manganese-porphyrin complexes can also be used as DNA-PKcs inhibitors. Such complexes have been documented to act as scavengers for oxidative species such as peroxynitrite, superoxide, and hydrogen peroxide. EUK-134 is one example of a synthetic manganese-porphyrin complex that can scavenge reactive oxygen species. As shown herein, EUK-134 is also an inhibitor of DNA-PKcs. The structure of EUK-134 is shown below.

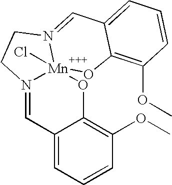

EUK-134 (Baker K. et al. (1998) J. Pharmacol. Exp. Ther. 284, 215-221, which is specifically incorporated herein by reference in its entirety) is a synthetic superoxide dismutase/catalase mimetic and a catalytic scavenger of reactive oxygen species. EUK-134 exhibits both superoxide dismutase (SOD) and catalase activities, catalytically eliminating both superoxide and hydrogen peroxide, respectively (Baudry M et al. Biochem Biophys Res Commun 192:964, 1993, which is specifically incorporated herein by reference in its entirety). EUK-134 consumes hydrogen peroxide in vitro. EUK-134 has been shown to prevent oxidative stress and attenuate brain damage in rats following systemic administration of kainic acid (Rong Y et al. Proc Natl Acad Sci 9:9897, 1999, which is specifically incorporated herein by reference in its entirety). EUK 134 showed protective effects in a rat stroke model, employing middle cerebral artery ligation (Baker K et al. J Pharmacol Exp Ther 284(1) 215-221, 1998, which is specifically incorporated herein by reference in its entirety).

Other inhibitors of DNA-PKcs that can be used in the invention include those disclosed by Hardcastle et al., J. Med. Chem. 48: 7829-46 (2005), which is specifically incorporated herein by reference in its entirety. For example, the chromen-4-one compounds described in Hardcastle et al. can be used as DNA-PKcs inhibitors in the practice of the invention. Hardcastle discloses 2-N-morpholino-8-dibenzofuranyl-chromen-4-one (NU7427) and 2-N-morpholino-8-dibenzothiophenyl-chromen-4-one (NU7441), which are excellent inhibitors of DNA-PKcs (IC₅₀ against DNA-PK=40 and 13 nM, respectively). The structures of the these inhibitors are shown below:

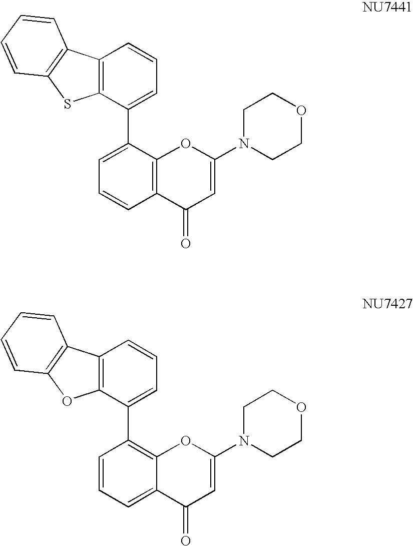

A structurally similar derivative of NU7427 that can be used as a DNA-PKcs inhibitor in the invention is shown below:

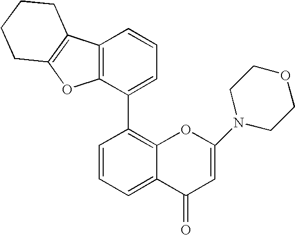

Hardcastle also discloses Compound 36 (8-(6′,7′,8′,9′-Tetrahydrodibenzothiophen-4′-yl)-2-N-morpholinochromen-4-one), which is shown below and which is also used in some of the experiments described this application.

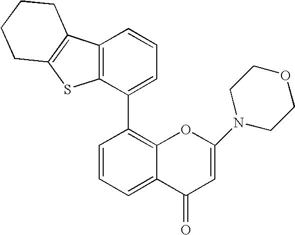

In addition, Hardcastle discloses the SU11752 compound as a useful DNA-PK inhibitor:

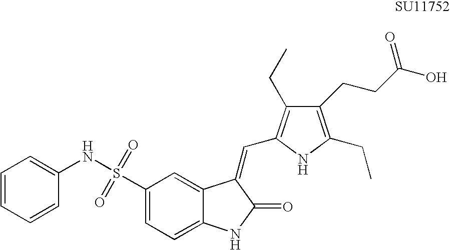

Other inhibitors of DNA-PKcs inhibitors that can be used in the invention include those disclosed by Leahy et al. J Bioorg. Med Chem Lett 14:6083-86 (2004), which is specifically incorporated herein by reference in its entirety. For example, an inhibitor disclosed by Leahy et al. with good activity include the NU7441 (8-dibenzothiophen-4-yl-2-morpholin-4-yl-chromen-4-one) compound with an IC₅₀ against DNA-PKcs of 14 nM, and having the following structure:

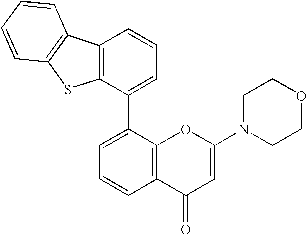

With its low molecular weight (only 413 Da), NU7441 is an attractive therapeutic agent for the treatment of neurological disorders such as stroke, Huntington's disease, Alzheimer's disease, Parkinson's diseases and ALS. DNA-PKcs inhibitors such as NU7441 may permeate the blood-brain barrier efficiently to ensure that the concentrations are sufficient to achieve the desired pharmacological effects.

Leahy discloses other DNA-PKcs inhibitors with similar structures that are useful in the invention, including those with an aryl or heteroaryl ring substituent (R₁₀) at the 6, 7 or 8 position of bicyclic ring, as shown below.

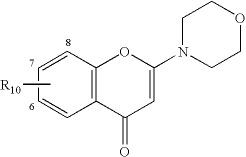

wherein R₁₀ is a mono-cyclic, bicyclic or tricyclic aryl or heteroaryl ring that can be substituted with hydroxy, alkoxy, or alkoxycarbonyl(acyl). Leahy also discloses useful DNA-PKcs inhibitors with the following structures, that are useful in the practice of the present invention.

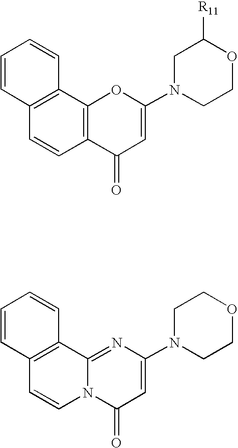

wherein R₁₁ is hydrogen (H) or methyl.

Other inhibitors of DNA-PKcs inhibitors that can be used in the invention include those disclosed by US Patent Application Publication No. 2007/0238731 A1, by Graeme Cameron Murray Smith et al. published on Oct. 11, 2007 (see also, Christmamm et al. Toxicology 193:3 (2003), both of which are specifically incorporated herein by reference in their entirety). Smith et al. (2007/0238731 A1) discloses several compounds having an IC50 for DNA-PKcs of less than 10 nM, (for example, compounds 5, 18, 23, 24, 25, 26 (KU-0060648), 29, 32, 51, 53, 60, 81, 82, 83, 84, 85, 86, 88, 90, 91 and 95 disclosed therein). While the present invention is directed to use of any of these compounds in the methods and compositions disclosed herein, only some of the structures for these compounds are shown below.

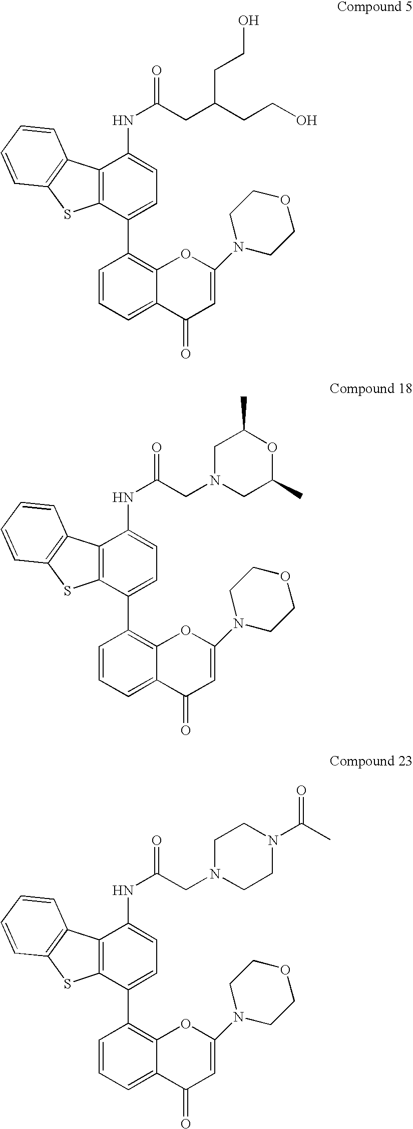

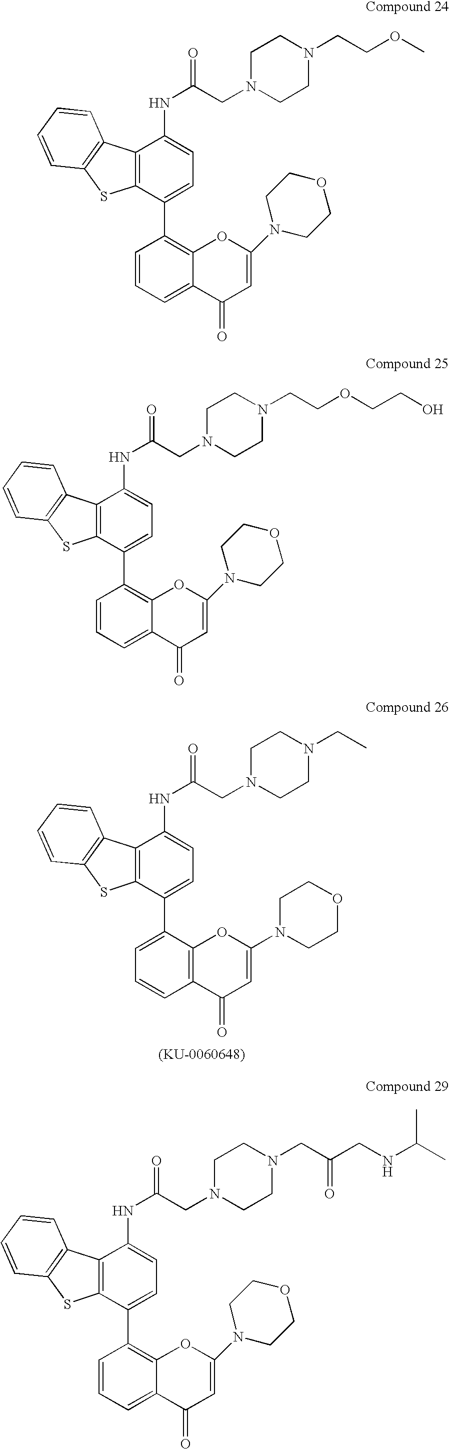

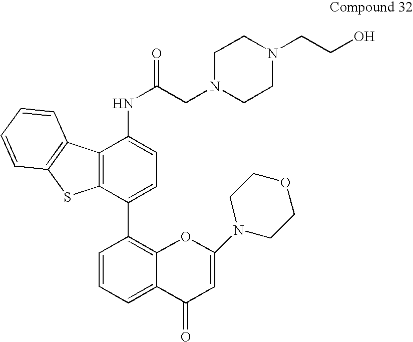

Additional compounds that can be used as DNA-PKcs inhibitors in the methods and/or compositions herein include the following compounds:

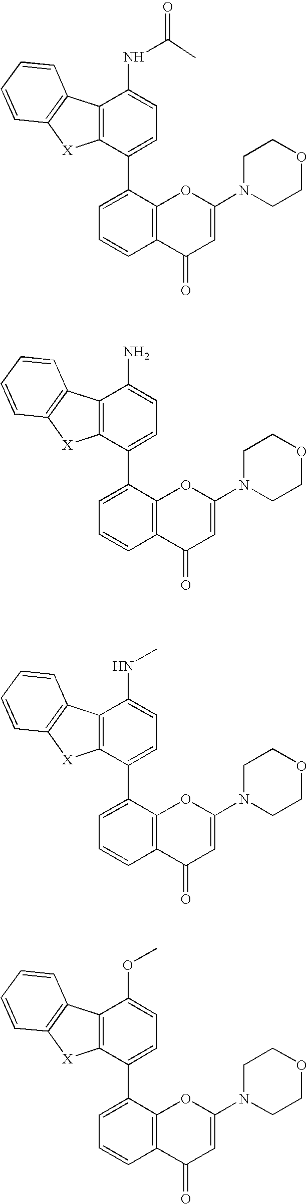

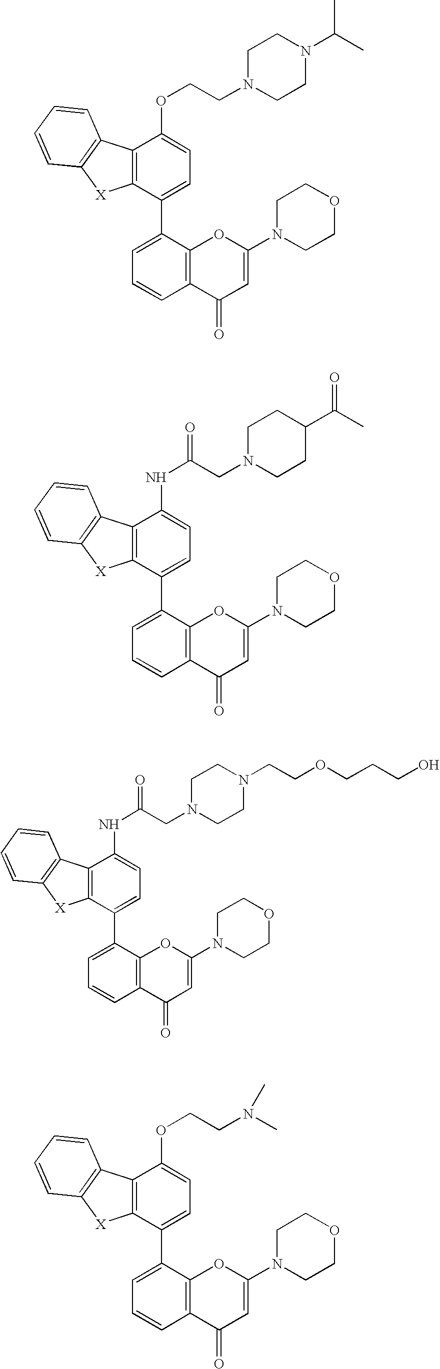

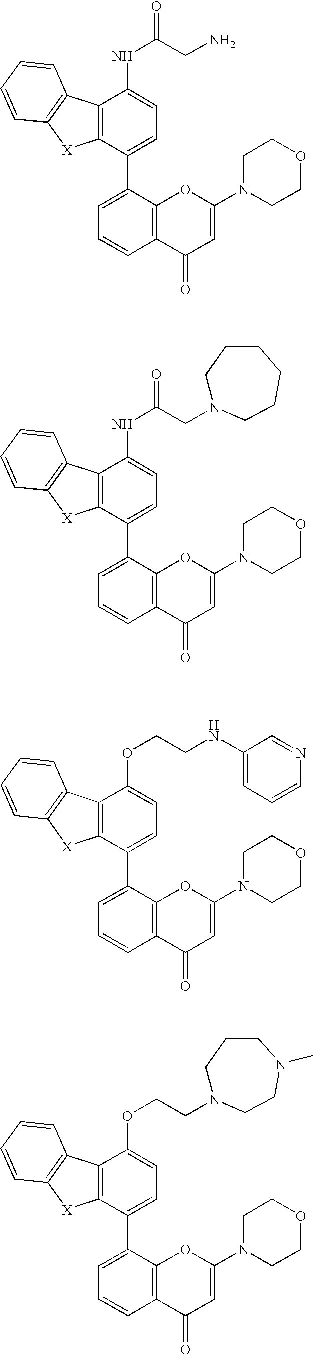

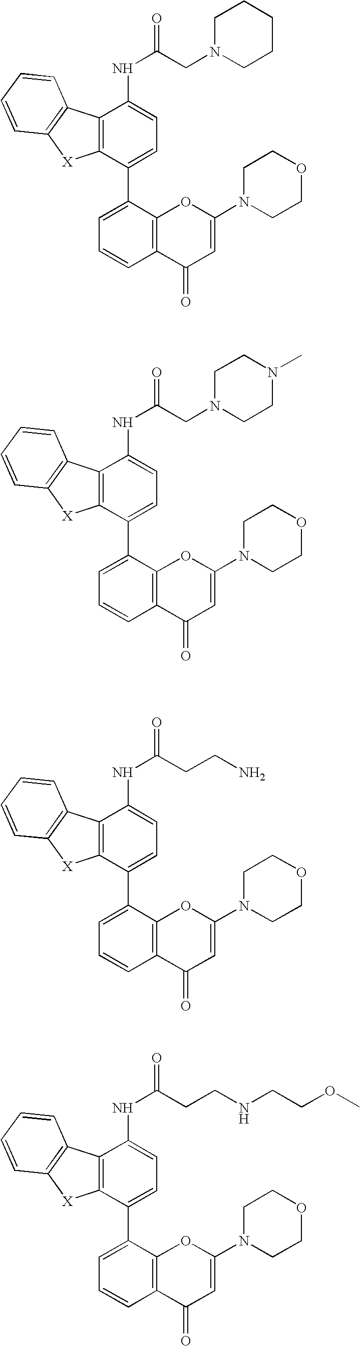

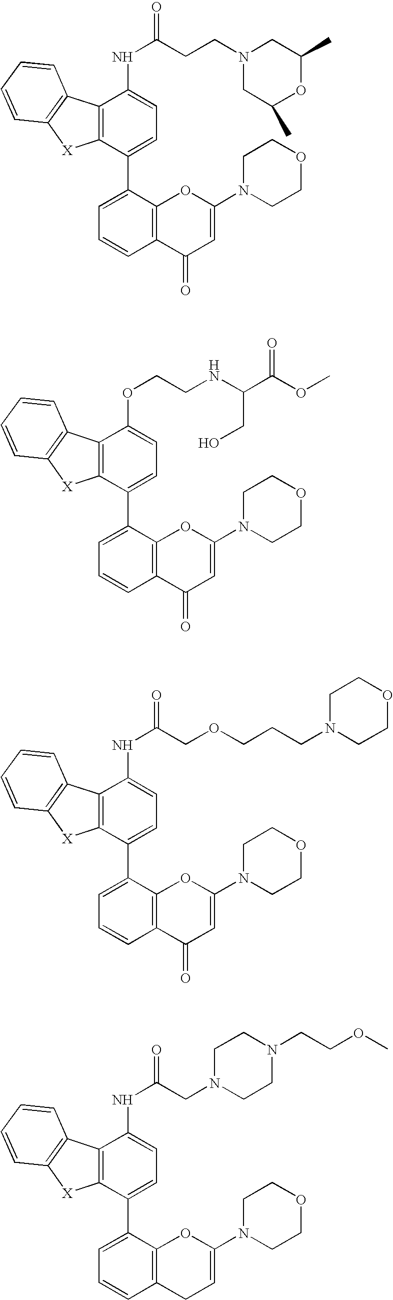

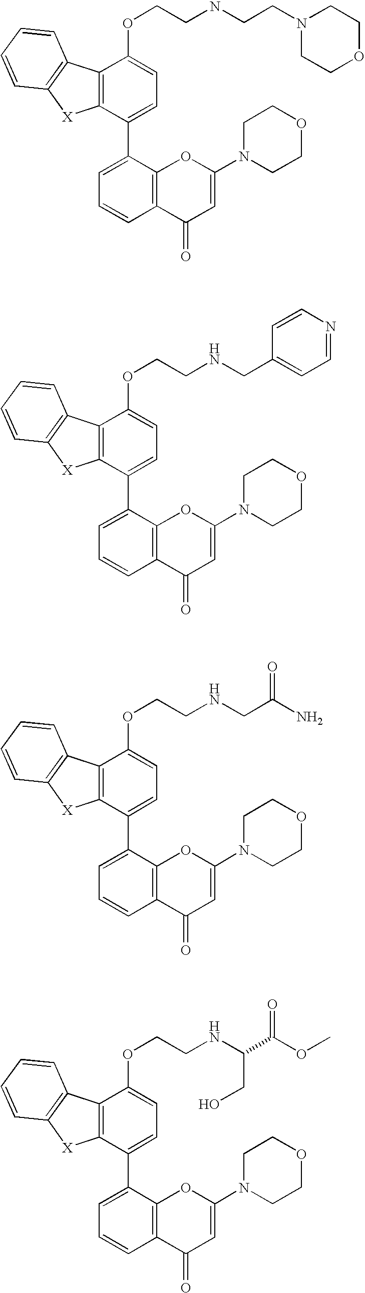

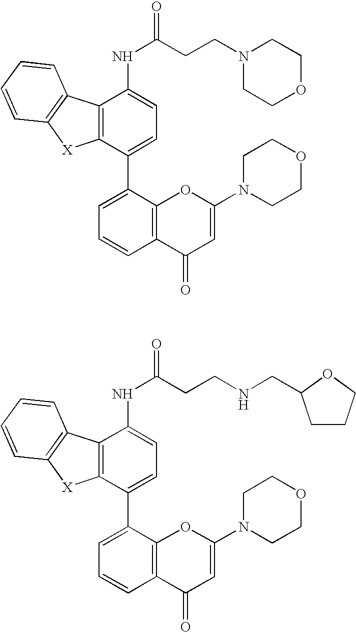

wherein X is a heteroatom. In some embodiments X in these compounds is oxygen (O) or sulfur (S).

Other inhibitors of DNA-PKcs inhibitors that can be used in the invention include those disclosed by Hollick, J. J. et al. Bioorg Med Chem Lett 13, 3083-6 (2003), which is specifically incorporated herein by reference in its entirety. Hollick et al. synthesized 6-aryl-2-morpholin-4-yl-4H-pyran-4-ones and 6-aryl-2-morpholin-4-yl-4H-thiopyran-4-ones bearing the 2-morpholin-4-yl group around the core structure of chromenone Ly294002 in order to evaluate DNA-PKcs inhibitor activities. 6-aryl-2-morpholin-4-yl-4H-thiopyran-4-ones bearing naphthyl or benzo[b]thienyl substituents at the 4′-position have been identified as potent DNA-PK inhibitors with IC(50) values in the 0.2-0.4 μM range. The pyran-4-one/thiopyran-4-one template was shown to retain the selectivity for DNA-PK shown for the benzo[h]chromenone scaffold (NU7026, IC50=0.23 μM). For example, compounds disclosed by Hollick that may be used in the invention can have the following structures:

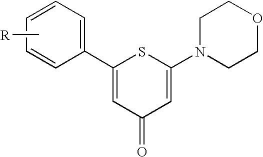

wherein R is halo, alkyl, alkoxy, aryl, or heteroaryl, wherein the alkyl, alkoxy, aryl or heteroaryl group can be substituted with one or more hydroxy, alkyl, alkenyl, alkylcarboxylate, or alkenylcarboxylate. Other compounds disclosed by Hollick et al. that can be used in the practice of the invention are disclosed in J. Med. Chem. 50: 1958-72 (2007), which is also specifically incorporated herein by reference in its entirety.

Other inhibitors of DNA-PKcs inhibitors that can be used in the invention include those disclosed by Griffin et al., J. Med. Chem. 48: 569-85 (2005), which is specifically incorporated herein by reference in its entirety. For example, one of the most potent compounds identified in this study is NU7163 (IC50=0.19 μM), 2-(2-methylmorpholine-4-yl)benzo[h]chromen-4-one, with the structure shown below.

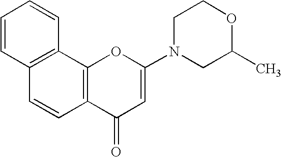

This NU7163 compound can be used as a DNA-PKcs inhibitor in the methods of the present invention. Other compounds disclosed by Griffin that can be used in the practice of the invention can have the following structures:

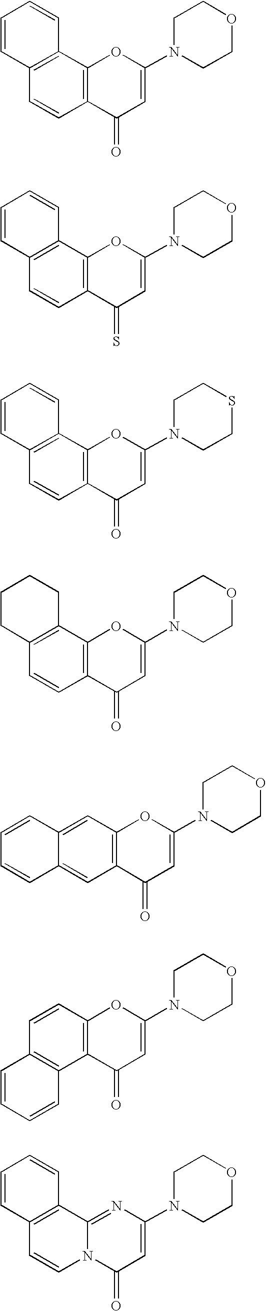

Manganese (III) tetrakis(4-benzoic acid)porphyrin (MnTBAP) is another manganese-porphyrin complex. MnTBAP is also a cell-permeable superoxide dismutase (SOD) mimetic and peroxynitrite scavenger. As shown herein, MnTBAP is also an inhibitor of DNA-PKcs. The structure of MnTBAP is provided below.

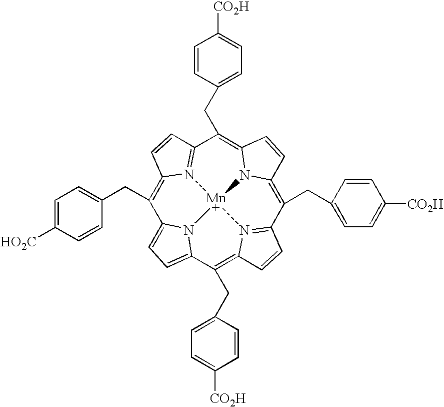

Metformin is an oral biguanide that is widely prescribed for type 2 diabetes (Kahn B B et al. Cell Metab. 1:15, 2005; Screaton R A Cell 119:61, 2004, both of which are specifically incorporated herein by reference in their entirety). Metformin increases glucose utilization and free fatty acid utilization, reduces hyperglycemia, lowers blood glucose and blood lipid contents, decreases hepatic gluconeogenesis and increases glucose uptake in skeletal muscle. Metformin acts through the stimulation of AMPK (AMP activated protein kinase) in peripheral tissues. Metformin has the following structure.

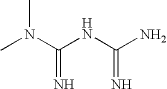

Dinitrophenol (DNP) is a cellular metabolic poison and an uncoupler. DNP uncouples oxidative phosphorylation by carrying protons across the mitochondrial membrane, leading to a rapid consumption of energy without generation of ATP. It separates the flow of electrons and the pumping of protons for ATP synthesis. Thus, the energy from electron transfer cannot be used for ATP synthesis. Low concentrations of DNP were shown to protect neurons against the toxicity of the amyloid-beta peptide (De Felice et al. FASEB J. 15:1297 (2001)). The structure of DNP is shown below.

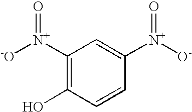

Other compounds that can act as inhibitors of DNA-PKcs or that can be used in combination with the DNA-PKcs inhibitors described above include thiazolidinediones (TZD), Epigallocatechin gallate (EGCG), IC60211 (2-hydroxy-4-morpholin-4-yl-benzaldehyde), IC86621 (a methyl ketone derivative of IC60211), IC486154, IC87102, IC87361, Wortmannin, LY294002, nucleic acids that can inhibit the expression and/or translation of DNA-PKcs, and the like. Thiazolidinediones or TZDs act by binding to peroxisome proliferator-activated receptors (PPARs), a group of receptors that reside inside the nucleus of a cell, specifically PPARγ (gamma). The normal ligands for these receptors are free fatty acids (FFAs). One example of a thiazolidinedione is troglitazone, (±)-[[4-[(3,4-dihydro-6-hydroxy-2,5,7,8-tetramethl-2H-1-benzopyran-2-yl)methoxy]phenyl]methyl]-2,4-thiazolidinedione, with the following structure.

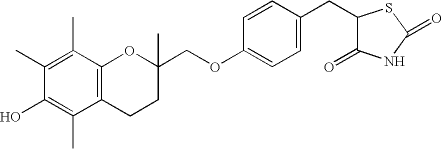

Epigallocatechin gallate (EGCG) is one of four major catechins in green tea and, according to the invention EGCG can be used as an inhibitor of DNA-PKcs. EGCG has the following structure.

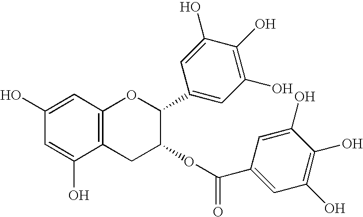

IC60211 (2-hydroxy-4-morpholin-4-yl-benzaldehyde), is another DNA-PK inhibitor having the following structure.

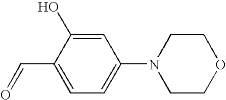

IC86621 is another DNA-PK inhibitor having the following structure.

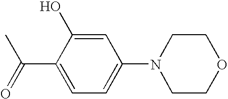

IC486154 is another DNA-PK inhibitor having the following structure.

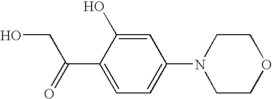

IC87102 is another DNA-PK inhibitor having the following structure.

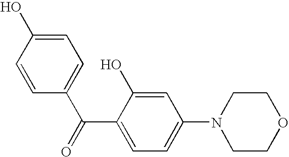

IC87361 is another DNA-PK inhibitor having the following structure.

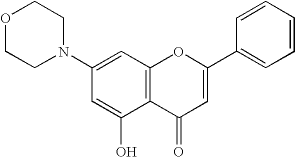

Wortmannin is another DNA-PK inhibitor having the following structure.

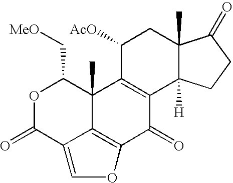

Other DNA-PKcs inhibitors that can be used include any of the inhibitors of DNA-PKcs described in Nutley et al., Br. J. Cancer 93: 1011-18 (2005), which is specifically incorporated herein by reference in its entirety. Examples of DNA-PKcs inhibitors disclosed by Nutley that may be used in the practice of the invention include NU7026 (chemical structure shown above); NU7031, 4-(morpholin-4-yl)-6-methoxy-1-benzopyran-2-one; NU7107, 2-((2S,6R)-2,6-dimethylmorpholin-4-yl)-pyrimido[2,1-a]isoquinolin-4-one; NU7199, 2-[bis-(2-hydroxyethyl)-amino]-benzo[H]chromen-4-one; and NU7200, 2-[2-(2-hydroxyethoxy)-ethylamino]-benzo[H]chromen-4-one.

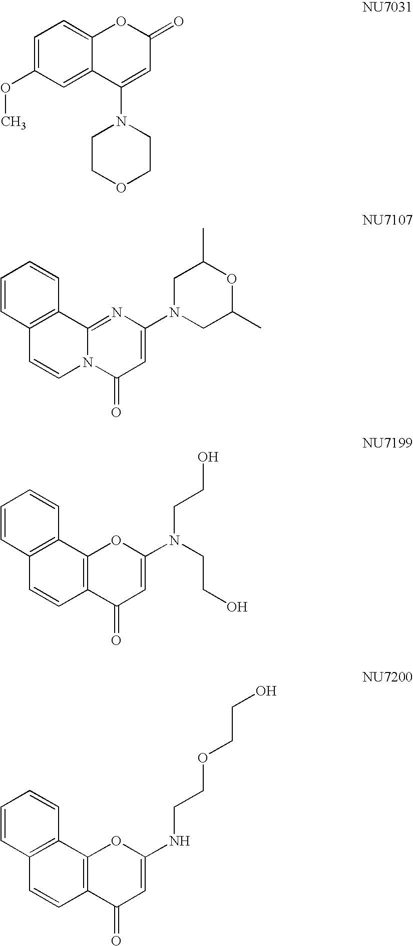

Other DNA-PKcs inhibitors that can be used include any of the inhibitors of DNA-PKcs described in Stockley et al., Bioorganic & Medicinal Chemistry Letters 11: 2837-41 (2001), which is specifically incorporated herein by reference in its entirety. One example of a DNA-PKcs inhibitor described by Stockley is the OK-1035 compound, which has the following structure:

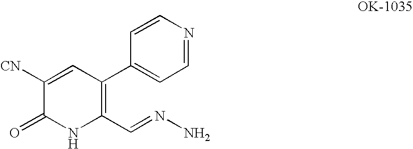

Other DNA-PKcs inhibitors that can be used include any of the inhibitors of DNA-PKcs described in Barbeau et al. (Org. Biomol. Chem. 5: 2670 (2007)), which is specifically incorporated herein by reference in its entirety. Examples of compounds disclosed by Barbeau et al. that can be used in the invention include 8-Substituted 2-morpholin-4-yl-quinolin-4-ones and 9-substituted 2-morpholin-4-yl-pyrido[1,2-a]pyrimidin-4-ones with aryl and heteroaryl groups.

Other DNA-PKcs inhibitors that can be used include AMA 37 (Aryl Morpholine Analog 37), 1-(2-Hydroxy-4-morpholin-4-yl-phenyl)-phenyl-methanone described in Willmore et al. Blood 103:4659 (2004) and Knight et al. Bioorg Med Chem 12:4749 (2004), both of which are specifically incorporated herein by reference in their entirety.

Vanillin (4-hydroxy-3-methoxybenzaldehyde) and its two derivatives, DMNB (4,5-dimethoxy-2-nitobenzaldehyde) and 3-iodo-4,5-dimethoxybenzaldehyde can also be used as DNA-PKcs inhibitors (Durant et al. Nucleic Acid Res 31:5501, 2003; Willmore et al. Blood 103:4659, 2004) in the practice of the invention.

In addition, according to the invention, nucleic acids that can inhibit the expression and/or translation of DNA-PKcs can also be used as inhibitors of DNA-PKcs. Such inhibitory nucleic acids can hybridize to a DNA-PKcs nucleic acid under intracellular or stringent conditions. The inhibitory nucleic acid is capable of reducing expression or translation of a nucleic acid encoding the DNA-PKcs. A nucleic acid encoding a DNA-PKcs may be genomic DNA as well as messenger RNA. It may be incorporated into a plasmid vector or viral DNA. It may be single strand or double strand, circular or linear. Examples of nucleic acids encoding DNA-PKcs are set forth in SEQ ID NO.2. DNA-PKcs nucleic acids may also be a fragment of the sequences set forth in SEQ ID NO:2 provided that the nucleic acids encode a biologically active DNA-PKcs polypeptide and/or a DNA-PKcs polypeptide capable of forming a DNA-PK.

An inhibitory nucleic acid is a polymer of ribose nucleotides or deoxyribose nucleotides having more than three nucleotides in length. An inhibitory nucleic acid may include naturally-occurring nucleotides; synthetic, modified, or pseudo-nucleotides such as phosphorothiolates; as well as nucleotides having a detectable label such as ³²P, biotin, fluorescent dye or digoxigenin. An inhibitory nucleic acid that can reduce the expression and/or activity of a DNA-PKcs nucleic acid, that is an inhibitory nucleic acid of the invention, may be completely complementary to the DNA-PKcs nucleic acid. Alternatively, some variability between the sequences may be permitted.

An inhibitory nucleic acid of the invention can hybridize to a DNA-PKcs nucleic acid under intracellular conditions or under stringent hybridization conditions. The inhibitory nucleic acids of the invention are sufficiently complementary to endogenous DNA-PKcs nucleic acids to inhibit expression of a DNA-PKcs nucleic acid under either or both conditions. Intracellular conditions refer to conditions such as temperature, pH and salt concentrations typically found inside a cell, e.g. a mammalian cell. One example of such a mammalian cell is the MCF7 cell described below, or any of the cell types where DNA-PKcs is or may be expressed.

Generally, stringent hybridization conditions are selected to be about 5° C. lower than the thermal melting point (Tₘ) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory nucleic acids that comprise, for example, 2, 3, 4, or 5 or more stretches of contiguous nucleotides that are precisely complementary to a DNA-PKcs coding sequence, each separated by a stretch of contiguous nucleotides that are not complementary to adjacent coding sequences, may inhibit the function of a DNA-PKcs nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a ribozyme or an antisense nucleic acid molecule.

The antisense nucleic acid molecule may be single or double stranded (e.g. a small interfering RNA (siRNA)), and may function in an enzyme-dependent manner or by steric blocking. Antisense molecules that function in an enzyme-dependent manner include forms dependent on RNase H activity to degrade target mRNA. These include single-stranded DNA, RNA and phosphorothioate molecules, as well as the double-stranded RNAi/siRNA system that involves target mRNA recognition through sense-antisense strand pairing followed by degradation of the target mRNA by the RNA-induced silencing complex. Steric blocking antisense, which are RNase-H independent, interferes with gene expression or other mRNA-dependent cellular processes by binding to a target mRNA and getting in the way of other processes. Steric blocking antisense includes 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce DNA-PKcs translation such that the level of DNA-PKcs polypeptide is reduced. siRNAs mediate post-transcriptional gene silencing in a sequence-specific manner. See, for example, http://www.ambion.com/techlib/hottopics/rnai/rnai_may2002_print.html (last retrieved May 10, 2006). Once incorporated into an RNA-induced silencing complex, siRNA mediate cleavage of the homologous endogenous mRNA transcript by guiding the complex to the homologous mRNA transcript, which is then cleaved by the complex. The siRNA may be homologous to any region of the DNA-PKcs mRNA transcript. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003). Typically, a target site that begin with AA, have 3′ UU overhangs for both the sense and antisense siRNA strands, and have an approximate 50% G/C content is selected. SiRNAs may be chemically synthesized, created by in vitro transcription, or expressed from an siRNA expression vector or a PCR expression cassette. See, e.g., http://www.ambion.com/techlib/tb/tb_{—}506html (last retrieved May 10, 2006).

When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO:4). SiRNAs also may be produced in vivo by cleavage of double-stranded RNA introduced directly or via a transgene or virus. Amplification by an RNA-dependent RNA polymerase may occur in some organisms.

An antisense inhibitory nucleic acid may also be used to specifically reduce DNA-PKcs expression, for example, by inhibiting transcription and/or translation. An antisense inhibitory nucleic acid is complementary to a sense nucleic acid encoding a DNA-PKcs. For example, it may be complementary to the coding strand of a double-stranded cDNA molecule or complementary to an mRNA sequence. It may be complementary to an entire coding strand or to only a portion thereof. It may also be complementary to all or part of the noncoding region of a nucleic acid encoding a DNA-PKcs. The non-coding region includes the 5′ and 3′ regions that flank the coding region, for example, the 5′ and 3′ untranslated sequences. An antisense inhibitory nucleic acid is generally at least six nucleotides in length, but may be about 8, 12, 15, 20, 25, 30, 35, 40, 45, or 50 nucleotides long. Longer inhibitory nucleic acids may also be used.

An antisense inhibitory nucleic acid may be prepared using methods known in the art, for example, by expression from an expression vector encoding the antisense inhibitory nucleic acid or from an expression cassette. Alternatively, it may be prepared by chemical synthesis using naturally-occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the antisense inhibitory nucleic acid and the sense nucleic acid.

Naturally-occurring nucleotides include the ribose or deoxyribose nucleotides adenosine, guanine, cytosine, thymine and uracil.

Examples of modified nucleotides include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, inhibitory nucleic acids of the invention may include modified nucleotides, as well as natural nucleotides such as combinations of ribose and deoxyribose nucleotides, and an antisense inhibitory nucleic acid of the invention may be of any length discussed above and that is complementary SEQ ID NO: 2.

An inhibitor of the invention may also be a ribozyme. A ribozyme is an RNA molecule with catalytic activity and is capable of cleaving a single-stranded nucleic acid such as an mRNA that has a homologous region. See, for example, Cech, Science 236: 1532-1539 (1987); Cech, Ann. Rev. Biochem. 59:543-568 (1990); Cech, Curr. Opin. Struct. Biol. 2: 605-609 (1992); Couture and Stinchcomb, Trends Genet. 12: 510-515 (1996). A ribozyme may be used to catalytically cleave a DNA-PKcs mRNA transcript and thereby inhibit translation of the mRNA. See, for example, Haseloff et al., U.S. Pat. No. 5,641,673. A ribozyme having specificity for a DNA-PKcs nucleic acid may be designed based on the nucleotide sequence of SEQ ID NO:2.

Methods of designing and constructing a ribozyme that can cleave an RNA molecule in trans in a highly sequence specific manner have been developed and described in the art. See, for example, Haseloff et al., Nature 334:585-591 (1988). A ribozyme may be targeted to a specific RNA by engineering a discrete “hybridization” region into the ribozyme. The hybridization region contains a sequence complementary to the target RNA that enables the ribozyme to specifically hybridize with the target. See, for example, Gerlach et al., EP 321,201. The target sequence may be a segment of about 5, 6, 7, 8, 9, 10, 12, 15, 20, or 50 contiguous nucleotides selected from a nucleotide sequence having SEQ ID NO:2. Longer complementary sequences may be used to increase the affinity of the hybridization sequence for the target.

The hybridizing and cleavage regions of the ribozyme can be integrally related; thus, upon hybridizing to the target RNA through the complementary regions, the catalytic region of the ribozyme can cleave the target. Thus, an existing ribozyme may be modified to target a DNA-PKcs nucleic acid of the invention by modifying the hybridization region of the ribozyme to include a sequence that is complementary to the target DNA-PKcs nucleic acid. Alternatively, an mRNA encoding a DNA-PKcs may be used to select a catalytic RNA having a specific ribonuclease activity from a pool of RNA molecules. See, for example, Bartel & Szostak, Science 261:1411-1418 (1993).

### Effects of DNA-PKcs Inhibition

According to the invention, inhibition of DNA-PKcs in a mammal reduces weight gain, increases the number of mitochondria, improves stamina, reduces blood pressure, increases thermogenesis, improves insulin sensitivity, improves insulin signaling, improves memory, improves learning, reduces depression, reduces anxiety and the like in the mammal.

In some embodiments, the present compositions and methods may be most beneficial when used with obese and/or middle-aged and/or older mammals. As used herein, an obese mammal weighs well above his or her normal weight. Mammals are generally obese if they are more than 20 percent over their ideal weight. That ideal weight must take into account the average weight for mammals of that species with consideration for the mammal's height (length), age, sex, and build. Obesity has been more precisely defined by the National Institutes of Health as a body mass index (BMI) of 30 and above. For humans, a BMI of 30 is about 30 pounds overweight. The BMI is a mammal's weight in kilograms divided by their height in meters squared. Since the BMI describes the body weight relative to height, it correlates strongly in adult mammals with the total body fat content. However, some very muscular people may have a high BMI without undue health risks.

As used herein, “middle-aged” means that a mammal is approximately in the middle 30-40% of its lifespan. Thus, the middle-aged mammal is older than about 30% of the average life-span of the mammal's species but younger than about 70% of the average life-span of the species. A middle-aged human is about 30 to about 65 years old.

An “older” mammal is a mammal in approximately the last third of the life-span for that species of mammals. Thus, for example, an older human is about 65 years or older.

DNA-PKcs Inhibition Mimics Calorie Restriction: As illustrated herein, DNA-PKcs inhibitors mimic calorie restriction. The inventors have also demonstrated that calorie restriction in vivo induces suppression of DNA-PKcs in primates. These results indicate that agents that inhibit DNA-PKcs activity function as mimetics of calorie restriction.

Thus, the invention provides DNA-PKcs inhibition as a method of inducing the physiological efforts of calorie restriction. Compounds that suppress the basal activity of DNA-PKcs function as calorie mimetics, and such DNA-PKcs inhibitors/antagonists produce the robust, multisystem effects associated with calorie restriction. Because DNA-PKcs inhibition mimics calorie restriction, such DNA-PKcs inhibition can beneficially treat various metabolic disorders, diseases and conditions such type II diabetes, obesity, cardiovascular diseases and dyslipidemia.

According to the present invention, DNA-PKcs plays an important role in regulating metabolism. When DNA-PKcs is active, mammals tend to have greater fat mass (see, e.g., FIG. 10) and lesser lean mass (data not shown). Cholesterol and leptin levels in obese mammals tend to be higher when DNA-PKcs is active. For example, wild type mice on a high fat diet have cholesterol levels of 3.4±1.3 nmol/l, whereas SCID mice maintained on a high fat diet have cholesterol levels of 2.7±0.3 nmol/l. Leptin levels, for example, in wild type mice that are maintained on a high fat diet are 57.8±20 nmol/l, whereas SCID mice maintained on a high fat diet have leptin levels of 17.4±13 nmol/l. Thus, loss of DNA-PKcs function reduces serum levels of cholesterol and leptin even when the mammal consumes a high-fat diet.

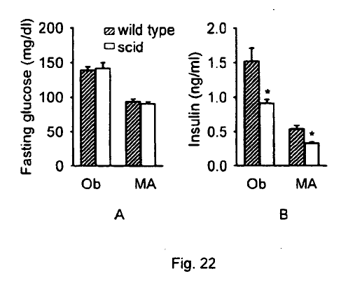

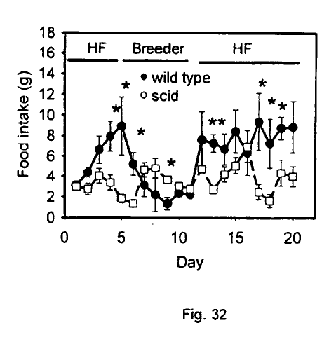

Loss of DNA-PKcs function also tends to make mammals more insulin sensitive (see, e.g., FIGS. 22-24). Inhibition of DNA-PKcs leads to greater energy usage, greater heat production (see, e.g., Table 1) and less high-fat diet binge eating (see, e.g., FIG. 32).

Such improvements in the metabolism of mammals upon inhibition/loss of DNA-PKcs function are also reflected in the expression patterns of key genes. As demonstrated herein, DNA-PKcs activation results in suppression of AMPK. However, DNA-PKcs deficiency, DNA-PKcs suppression by DNA-PKcs inhibitors/antagonists, or DNA-PKcs knock-down with DNA-PKcs siRNA has the opposite effect—inhibition of DNA-PKcs results in activation of AMPK in the absence of calorie deprivation.

AMPK (AMP activated protein kinase) is the primary regulator of the cellular response to lowered ATP levels in eukaryotic cells (Hardie and Carling, Eur. J. Biochem. 246:259, 1997). AMPK is activated by decrease in the energy state of the cells. Such AMPK activation is often measured by observing an increase in the cellular AMP/ATP ratio, which is a sensitive indicator of the energy state of the cell (Ruderman et al. Am. J. Physiol 276:E1, 1999). Thus, AMPK acts as an intracellular energy sensor.

AMPK is activated by the kinase LKB1. AMP directly binds to AMPK, making it a better substrate for LKB1 (Kahn et al. Cell Met 1:15, 2005). Alternately, AMPK is also activated by a Ca²⁺-dependent protein kinase without AMP (Hawley et al., Cell Met 2:9, 2005; Woods et al. Cell Met. 2:21, 2005).

AMPK activation enhances processes that increase ATP generation and inhibits processes that consume ATP. Processes that increase ATP generation include fatty acid oxidation, while processes that consume ATP include those involved in fatty acid, protein and cholesterol synthesis. The adipocyte-derived hormones leptin and adiponectin, as well as exercise, activate AMPK in skeletal muscle, stimulating fatty acid uptake and oxidation, glucose uptake and mitochondrial biogenesis. Adiponectin also activates AMPK in liver, increasing fatty acid oxidation and reducing gluconeogenesis, fatty acid synthesis and cholesterol synthesis. AMPK also inhibits insulin secretion from pancreatic beta cells. In adipocytes, AMPK activation inhibits fatty acid synthesis. Thus, activation of AMPK results in many beneficial metabolic effects (Minokoshi et al. Nature 415:339, 2002; Mu et al. Mol. Cell 7:1085, 2001; Shaw et al. Science 310:1642, 2005). The inventors show that DNA-PKcs deficiency causes suppression of AKT. These results suggest that DNA-PKcs inhibitors/antagonists and DNA-PKcs siRNA activate AMPK and suppress mTOR and AKT, thus exerting a broad range of beneficial effects of calorie restriction without restricting caloric intake.

The target of rapamycin (TOR) is a conserved Ser/Thr phosphatidylinositol kinase-related (PIKK) kinase that regulates cell growth and metabolism in response to environmental cues (Wullschleger et al. Cell 124:471, 2006). TOR integrates various signals to regulate cell growth and is a central controller of cell growth. When nutrients and other growth stimuli are present, cells upregulate macromolecular synthesis and thereby increase in size and mass. Conversely, cells respond to nutrient limitation or other types of stress by restraining macromolecular synthesis. In this manner, the mTOR pathway responds to growth factors, nutrients, energy and stress.

The mTOR pathway responds to insulin or insulin-like growth factor (IGFs) via the P13K pathway. Binding of insulin or IGFs to their receptors leads to recruitment and phosphorylation of the insulin receptor substrate (IRS), and subsequent recruitment of P13K, PDK1 and AKT, resulting in the phosphorylation and activation of AKT by PDK1. mTOR is connected to the insulin signaling pathway through the tuberous sclerosis proteins TSC1 and TSC2. TSC1 and TSC2 act as a heterodimer that negatively regulates mTOR signaling. TSC2 is phosphorylated and inactivated by AKT in response to insulin (Manning J Cell Biol 167:399, 2004; Manning et al. Genes Dev 19: 1773, 2005).

Cell growth and cell mass increase require a high level of cellular energy and a high rate of protein synthesis. mTOR senses the energy status of a cell through AMPK. Activation of AMPK inhibits mTOR signaling inhibiting phosphorylation of S6K1 and 4E-BP1. Activated AMPK directly phosphorylates TSC2, leading to the inhibition of mTOR signaling (Inoki et al. Cell 115:577, 2003).

Nutrient overload leads to obesity, insulin resistance and type 2 diabetes. Whereas increased fat intake is the major factor in diet-induced obesity and metabolic stress, increased protein consumption through elevated circulating amino acids also contribute to obesity and metabolic stress. Nutrients, especially amino acids, regulate mTOR signaling (Gao et al. Nat. Cell Biol 4:699, 2002; Wullschleger et al. Cell 124:471, 2006). While amino acids activate mTOR and phosphorylate its downstream effectors, ribosomal protein S6 kinase 1 (S6K1) and 4E-BP1, amino acid starvation results in a rapid dephosphorylation of the mTOR downstream effectors S6K1 and 4E-BP1 (Hay and Sonenberg Genes Dev. 18:1926, 2004). S6K1 and 4E-BP1 are critical regulators for protein translation and cell growth. Insulin-induced anabolic responses such as ribosome biogenesis and protein synthesis are dependent on nutritional state.

Insulin induces S6K1 activation which is initiated by insulin receptor autophosphorylation, and the recruitment and phosphorylation of IRS1 and IRS2 (White Mol Cell Biochem 182:3, 1998). This leads to the activation of AKT by PDK1 (Alessi et al. Curr Biol 7:261, 1997). AKT subsequently phosphorylates and inactivates TSC2, which leads to activation of mTOR and S6K1. In addition, recent findings also show that nutrients can activate mTOR/S6K1 independently of TSC1/2 (Nobukuni et al. Proc Natl Acad Sci USA 102:b14238, 2005; Smith et al. J Biol Chem 280:18717, 2005).

Although S6K1 is an effector of growth by insulin, S6K1 is also implicated in a negative feedback loop to suppress insulin signaling. In nutrient excess state, excess glucose or amino acids negatively affect insulin signaling through mTOR/S6K1 phosphorylation of IRS1 (Um et al. Nature 431:200, 2004; Um et al. Cell Metabolism 3:393, 2006). This phosphorylation of IRS1 by S6K1 then inhibits insulin signaling. Consistent with this, infusion of amino acids into humans leads to S6K1 activation and insulin resistance, demonstrating that S6K1 mediates insulin resistance in the face of nutrient excess. These studies show that nutrient overload leads to insulin resistance through activation of the mTOR/S6K1 signaling.

Starved cells degrade cytoplasmic contents including organelles, and thereby recycle macromolecules to ensure survival under starvation conditions. This protective catabolic process is called macroautophage. TOR/S6K1 negatively controls bulk protein degradation by macroautophagy (Blommaart et al. J Biol Chem 270: 2320, 1995; Dennis and Thomas Curr Biol 12:R269, 2002). TOR, in conjunction with AKT, also controls the turnover and trafficking of nutrient transporters, and thereby promotes uptake of nutrients such as glucose, amino acids and lipoprotein (Edinger and Thompson Mol Biol Cell 13:2276, 2002).

Recent studies have proposed that the anti-aging effects of calorie restriction may be, at least in part, due to reduction of core body temperature, suggesting that sustained reduction of core body temperature may prolong life span (Conti et al. Science 314:825, 2006).

Calorie restriction exhibits a robust and reproducible way of improving health and extending lifespan (Barger et al. Exp. Gerontol 38: 1343, 2003). These beneficial effects include lower insulin level, increased PGC-1 α a level, improved insulin sensitivity, lower core body temperature, decreased incidence of age-associated diseases including cancer, cardiovascular and cognitive disorders, slower age-related decline (Roth et al. Ann. NY Acad. Sci 1057:365, 2005; Baur and Sinclair Nat Rev Drug Discovery 5:493, 2006).

Recent work has demonstrated that calorie restriction also extends the life span of model organisms such as budding yeast, C. elegans and Drosophila. The mechanism by which calorie restriction extends life span-has been poorly understood. Studies from many different organisms indicate that oxygen radicals produced by mitochondria play a central role in promoting aging. Initially, it was thought that calorie restriction increases life span by decreasing the metabolic rate and the mitochondrial production of oxygen radicals. However, when normalized for lean body mass, calorie restriction does not decrease the metabolic rate but may actually increase it slightly. In addition, calorie restriction does actually decrease reactive oxygen species and this may slow aging.

The effect of calorie restriction on lifespan requires Sir2, an NAD-dependent histone deacetylase that was first identified in yeast as a silencer of telomeric chromatin. Decreased glucose concentration extends lifespan of yeast but this requires Sir2; although under some circumstances, the requirement for Sir2 can be bypassed with a Sir2 homolog Hst2 or an unknown pathway. The function of Sir2 in calorie restriction-mediated longevity is conserved evolutionarily. Calorie restriction increases the lifespan of Drosophila in a Sir2-dependent manner and increasing the dosage of Sir2 homolog in C. elegans and Drosophila increases lifespan. Sirt1, the mammalian homolog of Sir2, may also be involved in extending the lifespan. Sirt1 level increases with calorie restriction and it protects against p53-mediated cell senescence and NFκB-mediated inflammatory signaling. Suppression of NFκB-mediated signaling by over-expressing Sirt1 or by treating with resveratrol, which is thought to activate Sirt1, protects against neuronal death induced by amyloid beta peptides (Abeta), which are thought to cause Alzheimer disease. Sirt1 also suppresses adipogenesis and promotes loss of fat. Since reduction of fat is sufficient to extend murine lifespan, and inflammation promotes aging, increase in Sirt1 level or activity may also extend lifespan in mammals.

This information and the results described herein mean that inhibiting DNA-PKcs expression and/or activity in a mammal reduces weight gain, increases thermogenesis, and/or increases calorie consumption without calorie restriction or exercise.

DNA-PKcs Inhibition Improves Stamina: According to the present invention, DNA-PKcs plays an important role physical fitness and stamina. A striking characteristic of middle-aged SCID mice (which have a loss of function mutation in the DNA-PKcs gene) is exceptional physical fitness, endurance and youthfulness. Moreover, in SCID mice, the decline in the expression of genes involved in mitochondrial biogenesis, thermogenesis and fat burning, which occurs with obesity and aging in wild-type littermates, does not occur. As a consequence, SCID mice have increased mitochondrial content and thermogenesis and are resistant to diet-induced obesity. Their muscles contain more mitochondria, approximately 40% more ATP and are capable of running 2-3 times greater distances than wild-type littermates.

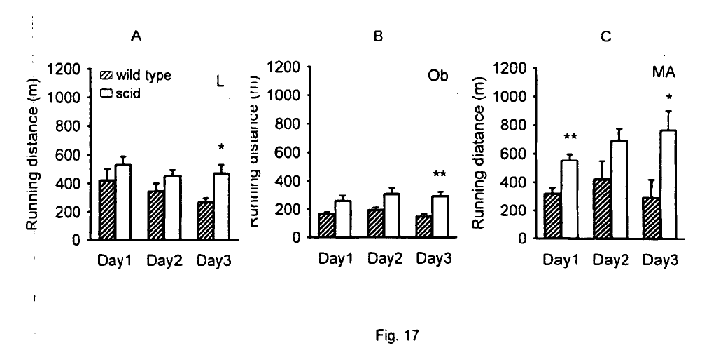

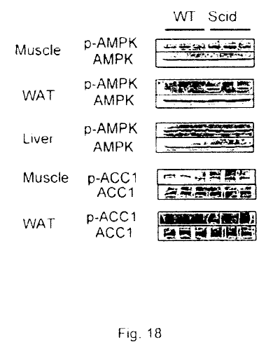

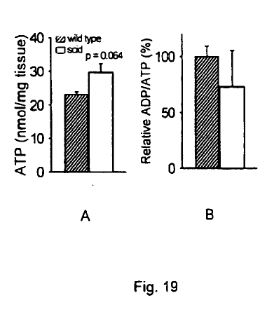

Thus, as demonstrated by the inventors, the relative mitochondrial DNA copy number in mammals with loss of DNA-PKcs function is about 2.5 times greater than in wild type mammals (see, e.g., FIG. 17). Mammals with loss of DNA-PKcs function have lower blood pressure. For example, wild type mice have an average blood pressure of 100±8 mm Hg, whereas the average blood pressure of SCID mice is 84±18 mm Hg. Use of oxygen, ATP levels and heat output are also greater in middle-aged mammals with loss of DNA-PKcs function (see, e.g., Table 1, FIG. 19). The running distance before exhaustion of mammals with loss of DNA-PKcs function is also 2-3 times greater than that observed for wild type mice (see, e.g., FIGS. 16-17).

Mitochondria are the principal energy sources in the cell, converting nutrients into energy (ATP) through respiration. In aerobic organisms like humans, oxygen is converted to water at the end of the respiratory chain in the mitochondria (Balaban et al. Cell 120:483, 2005). However, in this same mitochondria respiratory chain, oxygen is partially reduced to form superoxide. Superoxide is a radical that is a chemical species with an unpaired electron. Radicals are very reactive species, because electrons like to pair up to form stable bonds. Because of its radical character, superoxide is also called a “Reactive Oxygen Species (ROS)”. Thus, ROS are produced as a by-product of respiration.

The production of superoxide by the mitochondrial respiratory chain occurs continuously during normal aerobic metabolism. In addition to the mitochondrial respiratory chain, there are other endogenous sources of superoxide production. For example, when leukocytes encounter pathogens, they start to generate large amounts of superoxide. Additionally, glucose also increases intracellular ROS (Sakai et al. Biochem Biophys Res Commun 300:216, 2003; Amex et al. Biochim Biophys Acta 1271:165, 1995; Armann et al. Am J Transplan 7:38, 2007). Production of ROS is a physiological process in pancreatic beta cells and in these cells, ROS function as signaling molecules for insulin secretion (Bindokas et al. J Biol Chem 278:9796, 2003). Excessive ROS production in pancreatic beta cells can cause apoptosis of these cells.

The ROS formed by the mechanisms explained above can cause oxidative damage to various biological molecules, such as DNA, proteins and lipids, causing structural and functional damage. For example, oxidative damage to lipids in low-density lipoprotein plays an important role in atherosclerosis. Oxidative damage accumulates in human tissues with age and can causally contribute to a number of degenerative diseases including neurodegenerative diseases and ischemic-reperfusion diseases, heart disease and cancer.

Muscular wasting can also result from the accumulation of structural damages caused by a ROS imbalance induced by an increased oxidative metabolism in muscle fibers (Celegato et al. Proteomics 6:5303, 2006). Recently, the critical role of PGC-1 alpha (peroxisome proliferators-activated receptor γ coactivator) (Puigserver et al. Cell 92:829, 1998; Nature 423:550, 2003) in ROS metabolism, mitochondrial biogenesis and function has been also demonstrated (St-Pierre Cell 127:397, 2006). The decline in mitochondrial function with obesity and aging is coincident with the decline in the expression of PGC-1α (PPARγ coactivator-1α) and PGC-1β (PPARγ coactivator-1β).

In addition to promoting mitochondrial biogenesis and energy metabolism, PGC-1α is important for protecting neurons and muscle cells. PGC-1α protects against neuronal degeneration from ROS-induced oxidative damage (St-Pierre (2006)). Mice deficient in PGC-1α are very sensitive to neurodegnerative effects of MPTP and kainic acid, oxidative stressors affecting the substantia nigra and hippocampus, respectively. Increasing PGC-1α level protects neural cells in culture from oxidative-stressor-mediated death. Muscle atrophy that is induced by fasting, cancer cachexia, renal failure and denervation is accompanied by a drop in PGC-1α expression. Increased expression of PGC-1α protects against the mitochondrial decline and muscle atrophy. On the other hand, mice deficient in PGC-1β develop mitochondrial dysfunction and hepatic insulin resistance.

Heat is generated as by-product of energy expenditure. In a fully relaxed resting subject where energy expenditure equals the resting metabolic rate, the heat produced by the resting metabolism is called obligatory thermogenesis. However, the metabolic rate can be increased when exposed to cold or in response to food intake. Excessive caloric intake is thought to be sensed by the brain which triggers thermogenesis as a means of preventing obesity (Bachman et al. Science 297:843, 2002). The resulting heat production mechanism is called adaptive (or facultative) thermogenesis. Increased thermogenesis results in weight loss.

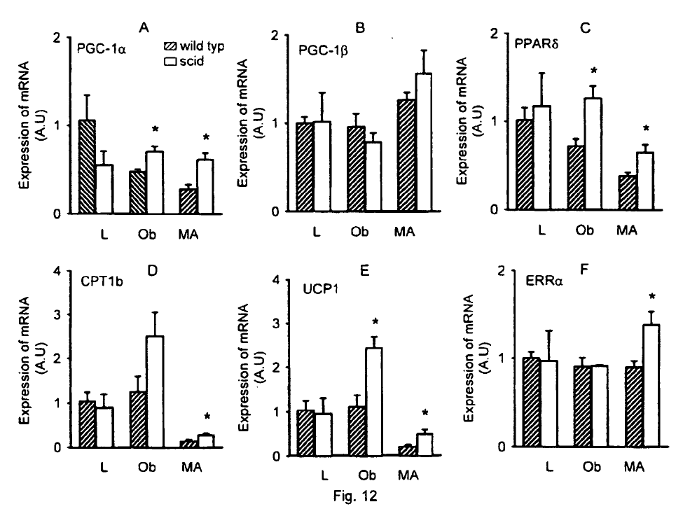

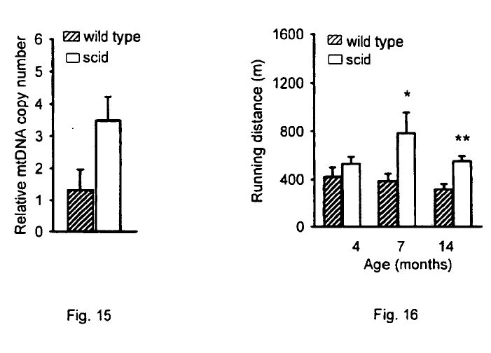

Brown adipose tissue (BAT) with its uncoupled mitochondrial respiration is the primary site of adaptive thermogenesis in small mammals and human newborns. Thermogenesis in BAT is regulated by the mitochondrial uncoupling proteins (UCP) (Thomas and Palmiter, Nature 387:94, 1997) and PGC-1 alpha, and occurs in response to cold and overeating (Rothwell et al. Nature 281:31, 1979; Brooks et al. Nature 286:274, 1980). As demonstrated herein inhibition of DNA-PKcs function increases PGC-1 alpha expression (see, e.g., FIG. 12-14), increases mitochondrial numbers (see, e.g., FIG. 15), improves physical fitness (see, e.g., FIG. 16-17), lowers blood pressure and has other beneficial physiological effects.

Accordingly, the invention relates to methods of lowering blood pressure, increasing stamina, improving mitochondrial function, biogeneis and increasing energy usage, and also provides method of improving brain function, reducing inflammation, reducing heart disease, and other age-related physiological problems.

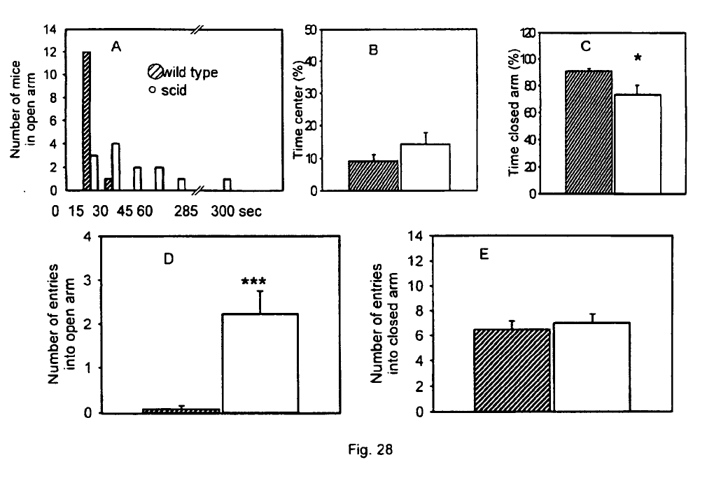

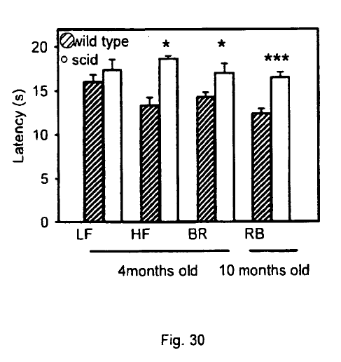

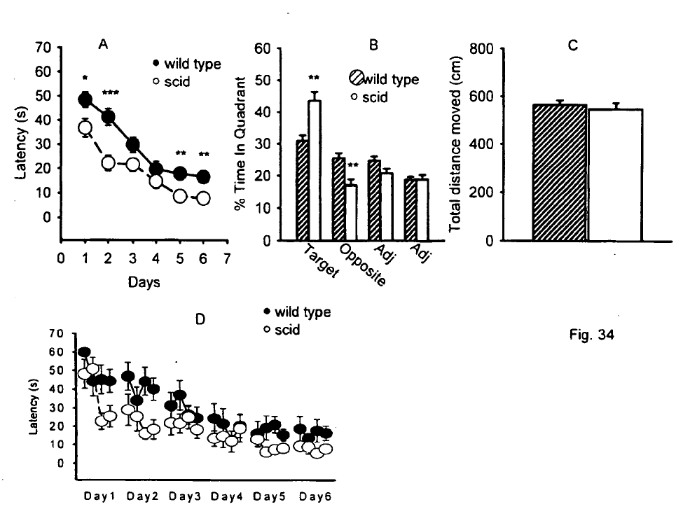

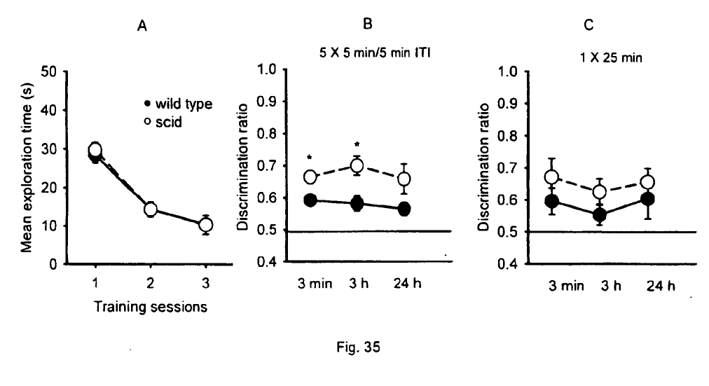

DNA-PKcs Inhibition Improves Memory and Reduces Anxiety: As illustrated herein, DNA-PKcs inhibition and/or loss leads to reduced anxiety-related behavior, (see, e.g., FIGS. 28-29, 33), greater resistance to pain (FIG. 30), improved memory (see, e.g., FIGS. 34-35), and less high-fat diet binge eating (FIG. 32).

According to the invention provides DNA-PKcs inhibition also leads to inhibition of target of rapamycin (TOR), which influences memory and aging in adults. During development, TOR may primarily control growth, whereas in the adult where there is relatively little growth, TOR appears to control aging and other aspects of nutrient-related, aging-related physiology. For example, rapamycin treatment in adults has been found to antagonize long-term memory formation (Tischmeyer et al. Eur J Nerusci 18:942, 2003; Casadio et al. Cell 99:221, 1999).

The connection with TOR indicates that DNA affects brain function. Data obtained by the inventors demonstrates that inhibition or loss of DNA-PKcs function leads to expression of higher levels of brain-derived neurotrophic factor (BDNF) in a mammal, which is associated with memory formation and suppression of anxiety and depression. Consistent with this, SCID mice actually do have better memory and reduced anxiety compared to wild-type controls. SCID mice are also resistant to stress-induced binge eating of high fat food.

Brain-derived neurotrophic factor (BDNF) is known to play a critical role in the synaptic plasticity for memory formation. BDNF is implicated in animal and human anxiety (F. Cirulli et al., Hippocampus 14, 802, 2004; M. Rios et al., Mol. Endocrinol. 15, 1748 (2001); U. E. Lang et al., Psychopharmacology 180, 95 (2005); E. Koronen et al., Mol. Cell. Neurosci. 26, 166 (2004)). Additionally, BDNF is also thought to have protective function in anxiety and depressive disorders (Heldt S A et al. Mol Psychiatry, 2007). Consistent with this, loss of one BDNF gene allele increased anxiety in serotonin transporter (SERT) knockout mice, implying that both BDNF and serotonergic systems interact in modulation of anxiety. In addition, these studies also reported that BDNF improves both short-term and long-term memory.

The process of memory formation requires three general stages (Tully T et al. Nat Rev Drug Discov 2:267, 2003). The first stage is learning that involves the initial perception of a new experience. The second state is a short-term memory formation. Short-term memory is labile and transient. With persistent repetition, however, the short-term memory is translated into a long term memory. Persistent, brief repetition causes frequent stimulation to monosynaptic excitatory pathways in the hippocampus and causes a sustained increase in the efficiency of synaptic transmission (Bliss et al. Nature 361:31, 1993; Bliss and Lomo J Physiol Lond 232:331, 1973). This effect is called long-term potentiation (LTP). LTP is a synaptic change in the chemical strength that alters neural connectivity. Such synaptic changes last from minutes to several days. While LTP is observed in all excitatory pathways in the hippocampus as well as in several other regions in the brain, LTP in the hippocampus is considered to play a major role for long term memory formation (Bliss and Collingridge Nature 361:31, 1993). Protein synthesis is thought to be required for the establishment of long-term memory but not for short term memory. Biochemically, long term memory starts from NMDA (N-methyl-D-aspartate)-receptor activation, which in turn activates transcriptional responses via phosphorylation of the transcription factor cyclic AMP-response element binding protein (CREB) (Bartsch et al. Cell 83:979, 1995; Alberini et al. Cell 76:1099, 1994). CREB is a transcription factor that is activated by neuronal activity. CREB isoforms function either as activators of gene expression or as repressors of the activators. CREB loss-of-function mutants have impairments in long-term memory, whereas CREB gain-of-function mutants show enhanced long-term memory. The protein kinase A (PKA, cyclic AMP-dependent kinase) and mitogen activated protein (MAP) kinase pathways play dominant roles in activation/phosphorylation of CREB (Xing J et al. Science 273:959, 1996; Martin K et al. Neuron 18:899, 1997; Impey S et al. Neuron 21:869, 1998). CREB regulates growth processes yielding synaptic changes (Frey U et al. Nature 385:533, 1997; Marth K et al. Cell 91:927, 1997). Thus, CREB is a key regulator that produces cellular changes in the strength and structure of synaptic connections between neurons underling the formation of long-term memory.

Thus, the invention relates to methods for improving brain function and avoiding neurological disorders such as Alzheimer's, Parkinson's, Huntingon's disease and Amyotropic lateral sclerosis (ALS) and Friedreich ataxia (FRDA) that are major protein conformational diseases associated with accumulation of abnormal proteins. In brain cells, aggregation of proteins in abnormal conformation leads to excessive production of ROS and brain injury.

DNA-PKcs Inhibition Reduces Inflammation: As described above, DNA-PKcs contributes to obesity, whereas inhibition of DNA-PKcs helps mammals resist obesity. Obesity is associated with metabolic and inflammatory stresses that affect glucose homeostasis.

JNK is a central kinase for inflammation and immune responses. In obese subjects, JNK1 is activated in insulin-responsive tissues such as fat, muscle and liver (Muoio and Newgard, Science 306:425, 2006; de Luca and Olefsky, Nat Med 12:41, 2006). JNK1 is activated by free fatty acids and inflammatory cytokines such as TNF alpha. These results indicate that inflammation and insulin-sensitivity and obesity are linked. Consistent with that, JNK1-deficient mice were resistant to high-fat diet-induced obesity and insulin resistance (Hirosumi et al. Nature 420:333, 2002; Ozcan et al. Science 306:457, 2004; Urano et al. Science 287:664, 2000). Thus, JNK1 is a crucial mediator of obesity and insulin resistance.

Many human illnesses have an inflammatory component. Inflammation is a normal response of the body to protect tissues from infection, injury or diseases. However, inflammation is also central to the pathology of arthritis, Crohn's disease, asthma, sepsis, psoriasis and many autoimmune diseases, neurodegenerative diseases and have a role in the development of metabolic disorders (type II diabetes, obesity and cardiovascular disease), cancer and aging. In recent years, the concept that activation of the proinflammatory pathway can be a mechanism for obesity-associated insulin resistance has emerged (de Luca and Olefsky Nat Med 12:41, 2006).

Tumor necrosis factor alpha (TNFα) is elevated in adipose tissue and blood from obese rodents, and blockade of TNF alpha improves insulin sensitivity. Interleukin (IL)-6 and monocyte chemoattractant protein (MCP-1) can also cause insulin resistance and elevated levels of TNF alpha, IL-6 and IL-8 have been reported in diabetic and insulin-resistant patients (Roytblat L, Rachinsky M, Fisher A, Greemberg L, Shapira Y, Douvdevani A, Gelman S. Obes Res. 2000, 8(9):673-5; Straczkowski M, Dzienis-Straczkowska S, Stepien A, Kowalska I, Szelachowska M, Kinalska I J Clin Endocrinol Metab. 2002, 87(10):4602-6; Hotamisligil G S, Peraldi P, Budavari A, Ellis R, White M F, Spiegelman B M. Science. 1996, 271(5249):665-8; Sartipy P, Loskutoff D J. Proc Natl Acad Sci USA. 2003, 100(12):7265-70; Hotamisligil G S, Arner P, Caro J F, Atkinson R L, Spiegelman B M. J Clin Invest. 1995, 95(5):2409-15).

DNA-PKcs Inhibition Can Reduce Heart/Vascular Disease: As described herein, inhibition of DNA-PKcs improves insulin sensitivity. According to the invention such insulin-sensitivity can reduce heart disease.

In particular, insulin resistance has far-flung negative effects on the development of heart disease. Elevated levels of the inflammatory marker C-reactive protein (CRP) are observed in patients with insulin resistance (de Luca and Olefsky Nat Med 12:41, 2006; Visser M, Bouter L M, McQuillan G M, Wener M H, Harris T B. JAMA. 1999, 282(22):2131-5). Furthermore, treatment with high-dose salicylate can inhibit Ikappa B kinase (IKK), a major kinase in the inflammatory pathway, and reverse glucose intolerance and insulin resistance in obese rodents (Yuan M, Konstantopoulos N, Lee J, Hansen L, Li Z W, Karin M, Shoelson S E. Science. 2001, 293(5535):1673-7).

Insulin resistance can promote endothelial dysfunction, and anti-TNF-alpha blockade yields a rapid improvement of endothelial function. Systemic inflammation, insulin resistance, and endothelial dysfunction have been implicated in the development of cardiovascular disease (de Luca and Olefsky Nat Med 12:41, 2006). The endothelium is responsible for the maintenance of vascular homeostasis. In physiological conditions, it acts keeping vascular tone, blood flow and membrane fluidity. The endothelial dysfunction occurring in the metabolic syndrome is the result of effects of the inflammatory cytokines such as TNF-alpha. Thus, the metabolic syndrome is considered a state of chronic inflammation accompanied of endothelial dysfunction, for example, causing an increased incidence of ischemic cardiovascular events, insulin resistance and high mortality. Therefore, therapies capable of reducing insulin resistance and inflammation can minimize the cardiovascular risk, type II diabetes and dyslipidemia due to metabolic syndrome.

Nitric oxide (NO) is an important signaling molecule in inflammation, blood vessel functions and macrophage activities (Moncada and Higgs N Engl J Med 329:2002, 1993). NO is synthesized from the amino acid L-arginine by the nitric oxide synthases. The synthesis of NO by vascular endothelium controls the vasodilator tone that is essential for the regulation of blood pressure. Calorie restriction or weight loss results in improvement of vascular tone.

Accordingly, because DNA-PKcs inhibition mimics calorie restriction, DNA-PKcs also improves vascular tone, improves metabolic parameters such as reduced plasma glucose, reduces circulating inflammatory cytokines, reduces oxidative stress and improves insulin sensitivity. See also, Zanetti et al. Atherosclerosis 175:253, 2004; Sciacqua et al. Diab Care 26:1673, 2003; Ziccardi et al. Circulation 105:804, 2002; Perticone et al. Diabetes 50:159, 2001. One of the benefits of calorie restriction is an improved endothelial function. CR is thought to show beneficial effects on endothelial function by enhancing eNOS expression and function.

In the central nervous system, NO is also a neurotransmitter (Nelson et al. Nature 378:383, 1995) that mediates many functions, including the memory formation. Consistent with that, VEGF, a growth factor that activates eNOS expression, promotes neurogenesis and as a result, improves memory, learning ability and cognition (Cao et al. Nature Genetics 36:827, 2004).

Angiogenesis is the growth of new capillary blood vessels. Two players in angiogenesis are VEGF (vascular endothelial growth factor) and Notch signaling pathways. Angiogenesis is required for embryogenesis, tissue repair after injury, growth and the female reproductive cycle. Angiogenesis also contributes to the pathology of cancer and a variety of chronic inflammatory diseases including psoriasis, diabetic retinopathy, rheumatoid arthritis, osteoarthritis, asthma and pulmonary fibrosis. For example, angiogenesis is required to support the growth of most solid tumors beyond a diameter of 2-3 mm. Recent studies show that angiogenesis inhibitors block tumor progression.

Based on these results, the inventors propose a theory that the function of DNA-PKcs in energy metabolism is inverse of the function of AMPK: AMPK, as a sensor of energy deficiency, increases insulin action and cellular ATP production, but DNA-PKcs, as a sensor of energy load, promotes mitochondrial decline and blocks the capacity for ATP production, resulting in the diversion of energy to fat storage. Thus, AMPK and DNA-PKcs have a “Yin and Yang” type of relationship in energy regulation. As such, the present invention suggests that DNA-PKcs inhibitors/antagonists would activate AMPK, resulting in mTOR inhibition and thereby mimicking the energy deprivation and calorie restriction status without restricting actual caloric intake.

Aging and obesity are also associated with increased inflammatory signaling. A number of diseases such as cancer, cardiovascular disease and diabetes, not to mention bona fide inflammatory diseases, are mediated by the IKK-NFκB-dependent inflammatory pathway. In the absence of DNA-PKcs, IKK-NFκB pathway is suppressed and inflammatory signaling is decreased in SCID tissues.

Another aspect of the invention is a method of using DNA-PKcs inhibitors/antagonists to treat diseases resulting from reactive oxygen species (ROS) production. The present inventors find that ROS activates DNA-PKcs, and glucose enhances ROS production to activate DNA-PKcs. In reverse, DNA-PKcs also plays a role in ROS production. Thus, this invention refers to the ROS-induced activation of DNA-PKcs, and nutrition, energy or calorie-induced activation of DNA-PKcs. Compounds that suppress ROS production and/or DNA-PKcs activities would be useful to treat or prevent various diseases that involve ROS production, for example, metabolic disorders, aging-related physical decline, ischemic-reperfusion diseases, stroke, injury, inflammatory diseases, neurodegenerative diseases and other degenerative diseases.

This invention provides a method of activating AMPK and suppressing mTOR and AKT in cells, tissues, in particular, insulin-sensitive tissues, or organisms using DNA-PKcs inhibitors/antagonists, their derivatives or DNA-PKcs siRNA, without imposing calorie restriction.

Another aspect of this invention is a method of increasing insulin sensitivity, insulin signaling, fatty acid uptake, glucose uptake, fatty acid oxidation and mitochondrial biogenesis, and reducing hyperglycemia, hepatic gluconeogenesis, fatty acid synthesis and cholesterol synthesis, using DNA-PKcs inhibitors/antagonists, their derivatives, or DNA-PKcs siRNA, without imposing calorie restriction. Another aspect of the invention is a method for identifying medicaments that enhance AMPK activation by their ability to block the ROS-, energy-, calorie- or nutrient-induced DNA-PKcs activation.

This invention provides a method of increasing autophage in cells, tissues or organisms by suppressing DNA-PKcs and subsequently suppressing mTOR, using DNA-PKcs inhibitors/antagonists, their derivatives, or DNA-PKcs siRNA. Cellular autophage is negatively regulated by mTOR and autophage is involved in degradation of proteins with abnormal conformation. Increasing autophage would be beneficial for preventing or treating degenerative neurological disorders that are associated with protein aggregates with abnormal conformation. This invention provides a method of treating or preventing neurodegenerative diseases including Alzheimer's, Parkinson's, Huntington diseases and ALS that are associated with protein aggregates using DNA-PKcs inhibitors/antagonists, their derivatives or DNA-PKcs siRNA.

The invention also relates to methods of treating, inhibiting and/or reducing aging-related physical decline, ischemic-reperfusion diseases, stroke, injury, inflammatory diseases, neurodegenerative diseases and other degenerative diseases.

Another aspect of the invention is a method of using antioxidants including Euk-134 that suppress DNA-PKcs activity to prevent or treat various diseases and conditions described in this invention.

Another aspect of the invention is a method of increasing transcription of genes important for thermogenesis and mitochondrial including PGC1-alpha, PPARδ, CPT1b, UCP1 and ERRα in the cells and systems in which such transcription occur using DNA-PKcs inhibitors/antagonists or DNA-PKcs RNAi. This invention relates to a method of improving thermogenesis, mitochondrial biogenesis and function, fat oxidation, metabolic rate, physical fitness, muscle function and endurance, and suppressing weight gain and fat accumulation, in particular, abdominal fat, by inhibiting DNA-PKcs activity using DNA-PKcs inhibitors/antagonists, their derivatives or DNA-PKcs RNAi.

This invention relates to a method of increasing eNOS and VEGF levels in cells, tissues or organisms by suppressing DNA-PKcs using DNA-PKcs inhibitors/antagonists or their derivatives. This invention provides a method of promoting angiogenesis in cells, tissues or organisms by suppressing DNA-PKcs using DNA-PKcs inhibitors/antagonists or its derivatives. This invention also relates to a method of decreasing blood pressure, increasing vasodilation and promoting wound healing using DNA-PKcs inhibitors/antagonists, their derivatives, or DNA-PKcs siRNA.

The invention also relates to a method of increasing the level of Sirt1 in cells, tissues or organisms, mimicking calorie restriction effects including longer lifespan of cells or organisms using DNA-PKcs inhibitors/antagonists or their derivatives, or DNA-PKcs siRNA.

This invention relates to a method of inhibiting IKK and NFκB, or stabilizing IκBα in cells, tissues or organisms, by suppressing DNA-PKcs using DNA-PKcs inhibitors/antagonists or their derivatives. This invention provides a method of treating or preventing a variety of inflammatory diseases using DNA-PKcs inhibitors/antagonists or their derivatives.

This invention also relates to a method of increasing eNOS, BDNF and CREB phosphorylation, and subsequent neurogenesis in brains cells and brain tissues, memory formation and improvement in cognitive abilities, by suppressing DNA-PKcs. Thus, this invention provides a method of treating or preventing stroke, anxiety, depression, memory loss and cognitive disorders using DNA-PKcs inhibitors/antagonists, their derivatives, or DNA-PKcs siRNA.

This invention relates to a method of reducing pain sensation by suppressing DNA-PKcs using DNA-PKcs inhibitors/antagonists, their derivatives, or DNA-PKcs siRNA.

This invention relates to a method of modulating serotonergic pathway, in particular, by inhibiting serotonin reuptake, in brain cells or brain tissues, by suppressing DNA-PKcs using DNA-PKcs inhibitors/antagonists or their derivatives, or DNA-PKcs siRNA. This invention also provides a method of treating stress-induced eating disorders including binge eating, anorexia nervosa and bulimia, mood disorders, anxiety and depression.

Another aspect of the invention is a description of diagnostic procedures for detecting diseases or monitoring progression of diseases that are associated with DNA-PKcs activation as a function of DNA-PKcs autophosphorylation and activation. Autophosphorylation of DNA-PKcs is essential for DNA-PKcs activities. Autophosphorylation of DNA-PKcs is suppressed by a protein phosphatase 5 (PP5) (Wechsler T et al. Proc Natl Acad Sci USA 101:1247, 2004). PP5 interacts with DNA-PKcs and dephosphorylates DA-PKcs. It is expected that PP5 activators/agonists would suppress DNA-PKcs autophosphorylation, functioning as DNA-PKcs inhibitors/antagonists. PP5 activators/agonists would be useful to treat or present various diseases described in this invention.

The present application describes newly identified signaling pathways of DNA-PKcs in energy regulation and brain function, and compounds or methods to antagonize or inhibit the DNA-PKcs activities. As a result of this invention, it is now possible to suppress the activity of DNA-PKcs to alter or modify the energy regulation mechanisms and brain functions using DNA-PKcs inhibitors and antagonists, for example, and of the compounds disclosed herein including NU7026 and Compound 36. These compounds include, but are not limited to, NU7026, Compound 36 Euk-134, resveratrol, metformin, TZD, DNP, MnTBAP and anti-oxidants, their derivatives, or any combination of these and the other compounds disclosed herein. This invention provides for methods for suppressing DNA-PKcs using DNA-PKcs inhibitors/antagonists that may be applied to treating disease conditions caused by DNA-PKcs activation. These diseases include metabolic disorders such as type II diabetes, obesity, cardiovascular diseases and dyslipidemia, aging-related physical decline, memory loss, ischemic-reperfusion diseases, stroke, injury, inflammatory diseases, neurodegenerative diseases, eating disorders, anxiety, depression, mitochondrial diseases and other degenerative diseases.

### Compositions and Formulations

In one embodiment, the invention provides a pharmaceutical composition comprising an inhibitor or antagonist of DNA-PKcs. To prepare such a pharmaceutical composition, an inhibitor or antagonist of the invention is synthesized or otherwise obtained, purified as necessary or desired, and optionally lyophilized and/or stabilized. The composition is then prepared by mixing the inhibitor with a carrier (e.g., a pharmaceutically acceptable carrier), adjusting it to the appropriate concentration and then combined with other agent(s).

By “pharmaceutically acceptable” it is meant a carrier, diluent, excipient, and/or salt that is compatible with the other ingredients of the formulation, and not deleterious to the recipient thereof.

The inhibitors of the invention can be used in a therapeutically effective amount. The term “therapeutically-effective amount” as used herein, pertains to that amount of an active compound (e.g., DNA-PK inhibitor), or a material, composition or dosage from comprising an active compound, which is effective for producing some desired therapeutic effect, commensurate with a reasonable benefit/risk ratio.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target tissue or physiological system being treated, and the subject being treated. Single or multiple administrations can be carried out with the dose level and pattern being selected by the treating physician.

In general, a suitable dose of the active compound is in the range of about 10 μg to about 250 mg per kilogram body weight of the subject per day. In some embodiments, the dose is about 100 μg to about 100 mg per kilogram body weight per day. In other embodiments, the dose is about 1 mg to about 50 mg per kilogram body weight.

Pharmaceutical formulations containing a therapeutic inhibitor of the invention can be prepared by procedures known in the art using well-known and readily available ingredients. For example, the inhibitor can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone.

Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For oral administration, an inhibitor may be present as a powder, a granular formulation, a solution, a suspension, an emulsion or in a natural or synthetic polymer or resin for ingestion of the active ingredients from a chewing gum. The inhibitor may also be presented as a bolus, electuary or paste. The formulations may, where appropriate, be conveniently presented in discrete unit dosage forms and may be prepared by any of the methods well known to the pharmaceutical arts including the step of mixing the therapeutic agent with liquid carriers, solid matrices, semi-solid carriers, finely divided solid carriers or combinations thereof, and then, if necessary, introducing or shaping the product into the desired delivery system. The total active ingredients in such formulations comprise from 0.1 to 99.9% by weight of the formulation.

In many embodiments, the inhibitors of the invention are administered as tablets and/or capsules. Tablets or caplets containing the inhibitors of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one inhibitor of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more inhibitors of the invention are designed to resist disintegration in the stomach and dissolve in the more neutral to alkaline environment of the duodenum.

Orally administered inhibitors of the invention can also be formulated for sustained release. In this case, an inhibitor of the invention can be coated, micro-encapsulated (see WO 94/07529, and U.S. Pat. No. 4,962,091), or otherwise placed within a sustained delivery device. A sustained-release formulation can be designed to release the inhibitor, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

An inhibitor of the invention can also be formulated as elixirs or solutions for convenient oral administration or as solutions appropriate for parenteral administration, for instance by intramuscular, subcutaneous, intraperitoneal or intravenous routes. A pharmaceutical formulation of an inhibitor of the invention can also take the form of an aqueous or anhydrous solution or dispersion, or alternatively the form of an emulsion or suspension or salve.

Thus, an inhibitor may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The inhibitors and other ingredients may form suspensions, solutions, or emulsions in oily or aqueous vehicles, and may contain formulatory agents such as suspending, stabilizing and/or dispersing agents. Alternatively, the inhibitors and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add other ingredients such as antioxidants, surfactants, preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

For topical administration, the inhibitors may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Thus, in one embodiment, an inhibitor of the invention can be formulated as a cream to be applied topically. Other conventional forms for this purpose include wound dressings, coated bandages or other polymer coverings, ointments, creams, lotions, pastes, jellies, sprays, and aerosols. Thus, the inhibitors of the invention can be delivered via patches or bandages for dermal administration. Alternatively, the inhibitor can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The inhibitors can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the inhibitors in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The inhibitors may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The inhibitors of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of the viral infection. Any statistically significant attenuation of one or more symptoms of the infection that has been treated pursuant to the method of the present invention is considered to be a treatment of such infection within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered orally or with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Inhibitors of the present invention can also be administered in an aqueous solution when administered in an oral, aerosol or inhaled form. Thus, other aerosol pharmaceutical formulations may comprise, for example, a physiologically acceptable buffered saline solution containing between about 0.1 mg/mL and about 100 mg/mL of one or more of the inhibitors of the present invention specific for the indication or disease to be treated. Dry aerosol in the form of finely divided solid inhibitor or nucleic acid particles that are not dissolved or suspended in a liquid are also useful in the practice of the present invention. Inhibitors of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the inhibitors of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

An inhibitor of the invention may also be used in combination with one or more known therapeutic agents, for example, a pain reliever; a vitamin; an antioxidant; an antibacterial agent; an anti-cancer agent; an anti-inflammatory agent; an antihistamine; a bronchodilator and appropriate combinations thereof, whether for the conditions described or some other condition.

### Kits and Articles of Manufacture

In one embodiment, the invention provides an article of manufacture that includes a pharmaceutical composition containing an inhibitor of the invention for any of the uses and methods of the invention. Such articles may be a useful device such as a sustained release device, bandage, transdermal patch or a similar device. The device holds a therapeutically effective amount of a pharmaceutical composition. The device may be packaged in a kit along with instructions for using the pharmaceutical composition for any of the uses or methods described herein. The pharmaceutical composition includes at least one inhibitor of the present invention, in a therapeutically effective amount such that the use or method is accomplished.

The invention is further illustrated by the following non-limiting Examples.

### Example 1

### DNA-PKcs is Activated by Exogenous Sources of ROS

While basal levels of reactive oxygen species (ROS) are normally produced in cells during ATP production, ROS are also generated by genotoxic stress such as ionizing radiation. Ionizing radiation generates double-stranded breaks in DNA and oxygen radicals, and is commonly used to activate DNA-PKcs. However, the possible effect of reactive oxygen species on DNA-PKcs activity and the possible role of DNA-PK in energy metabolism, obesity and aging are unknown. This Example describes experiments designed to test the effects of reactive oxygen species on DNA-PKcs activity.

### Methods

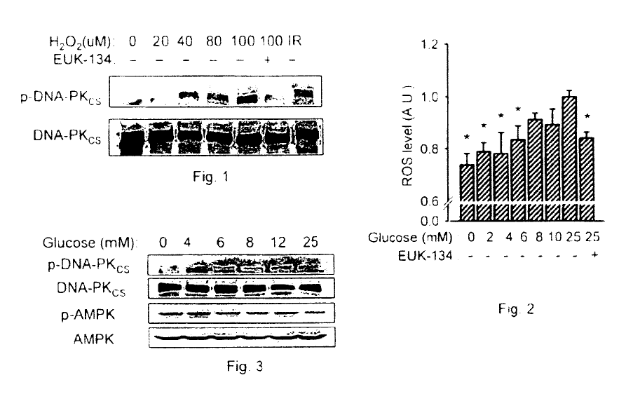

MCF7 cells were obtained from the ATCC. MCF7 cells in G₀ were treated with varying doses of H₂O₂ (FIG. 1). The cells were examined while in the G₀ phase of their life cycle to mimic the post-mitotic state of cells in vivo. Low passage MCF-7 cells in G₀ were exposed to varying concentrations of H₂O₂ for 60 minutes with or without Euk-134 or ionizing radiation (5 Gy). For Western blot analysis of DNA-PKcs activation and the activation of other signaling pathways in this study, the following antibodies were used: ACC1 (Cell Signaling); p-ACC1 (Upstate); AMP-activated protein kinase (AMPK; Cell Signaling); p-AMPK(T172) (Cell Signaling); DNA-PK (Lab Vision); p53 (Novocastra); p-DNA-PK(S2056) (Abeam); p-p53(S15) (Cell Signaling); γ-H₂AX (Upstate); 53BP1 (Novus Biologicals).

Activation of DNA-PKcs was visualized by immunoblotting cell lysates with antibody specific for phosphorylated Ser2056, which is autophosphorylated during DNA-PKcs activation (Chen et al., J. Biol. Chem. 280: 14709-15 (2005)) (FIG. 1). Total DNA-PKcs levels did not change with H₂O₂. Instead, H₂O₂ increased DNA-PKcs activation, and such activation was abrogated by the antioxidant Euk-134 (FIG. 1), a potent synthetic superoxide dismutase and catalase mimetic (Chatterjee, Am. J. Nephrol. 24: 165-77 (2004)).

MCF7 cells were then cultured in 25 mM glucose. Aliquots of the cultured MCF7 cells were treated with ionizing radiation and/or the DNA-PKcs-specific inhibitor NU7026. Treatment with glucose and ionizing radiation increased DNA-PKcs phosphorylation (data not shown). The DNA-PKcs-specific inhibitor NU7026 was able to depress the induction of DNA-PKcs activation by ionizing radiation (data not shown). The basal activity of DNA-PKcs in MCF-7 cells in 25 mM glucose was detectable even in the absence of ionizing radiation, but this basal activity was suppressed with the DNA-PKcs-specific inhibitor NU7026 (data not shown).

### Example 2

### ROS Production is Increased with Glucose

The observation that DNA-PKcs can be activated by exogenous sources of reactive oxygen species and that DNA-PKcs in cells cultured in 25 mM glucose is already activated (Example 1) prompted studies on whether the endogenous production of reactive oxygen species could regulate the activity of DNA-PKcs. Since energy metabolism is the major source of basal reactive oxygen species, MCF7 cells were first examined to ascertain whether glucose can increase production of reactive oxygen species. Subsequent tests, described in Example 3 and Example 4, were performed to ascertain whether glucose can activate DNA-PKcs in MCF7 cells.

Measurement of intracellular reactive oxygen species was based on changes in the fluorescence intensity of redox-sensitive fluorescent probes, including CM-H₂DCFDA. CM-H₂DCFDA is a probe for intracellular hydrogen peroxide (Jou M J et al. J. Biomed Sci 9:507 (2002)). CM-H₂DCFDA rapidly diffuses into cells, reacts with intracellular glutathione and thiols, and yields a fluorescent product that is retained inside the cell (Shanker G et al. Mol Brain Res 128:48, 2004). The fluorescence intensity of CM-H₂DCFDA is therefore indicative of the amount of intracellular H₂O₂.

FIG. 2 shows the reactive oxygen species levels in MCF-7 cells exposed to varying concentrations of glucose (3 hr), as measured by CM-H₂DCFDA (Invitrogen) according to the manufacturer's protocol. Briefly, cells were incubated with 10 uM CM-H₂DCFDA for 30 min at 37° C. The cells were then excited at the peak excitation wavelength for CM-H₂DCFDA (485 nm) and emissions at 535 nm were measured with Wallac Victor multilable counter (Perkin Elmer). For glucose treatment, the total sugar content in the media was kept constant by supplementing with mannitol, which is not metabolized.

As shown in FIG. 2, the reactive oxygen species production increased with glucose concentration (0-25 mM) in MCF7 cells and this glucose-induced reactive oxygen species production was partially suppressed with Euk-134 (FIG. 2; see also, FIG. 1).

### Example 3

### DNA-PKcs is Activated by Glucose

DNA-PKcs activation was then examined in MCF7 cells exposed to media containing 0-25 mM glucose for 3 hours. The basal activity of DNA-PKcs increased with increasing glucose concentration (FIG. 3). As expected, the activity of 5′-AMP kinase (AMPK)(Hardie et al., Eur. J. Biochem. 246: 259-73 (1997)), which senses energy depletion through 5′-AMP, decreased with increasing glucose concentration (FIG. 3).

### Example 4

### ROS Production is Suppressed by DNP, Troglitazone, Euk-134, MnTBAP, Resveratrol and NU7026 In Vitro

DNA-PKcs may be activated by endogenous reactive oxygen species (ROS) that is normally produced through energy metabolism, and ROS may mediate its harmful effects, at least in part, by activating DNA-PKcs. Thus, it is important to know how to suppress the basal activity of DNA-PKcs in cells. Tests were therefore performed to ascertain: 1) whether known compounds that decrease ROS production or inhibit oxidative phosphorylation such as superoxide dismutase mimetics Euk-134 and MnTBAP, mitochondrial uncoupler 2,4-dinitrophenol (DNP), Troglitazone and Resveratrol would suppress ROS production in MCF7 cells; 2) whether the DNA-PKcs inhibitor, NU7026, would also suppress ROS production in the same assay; and if so, 3) whether these compounds suppress DNA-PKcs in cells. Several experiments addressing these issues are described in this Example and in Example 5.

MCF-7 cells were obtained from ATCC and grown as recommended. Confluent MCF-7 cells were incubated with serum-free DMEM medium for 3-12 h before the experiment. MCF-7 cells were then treated with DNA-PK inhibitor NU 7026 (5 μM; Calbiochem) for 12 h, DNP (200 μM; Sigma) for 10 min, Troglitazone (30 μM) for 4 hr, Euk-134 (5 μM; Eukarion) for 3 h, MnTBAP (14 μM; Calbiochem) for 3 h and Resveratrol.

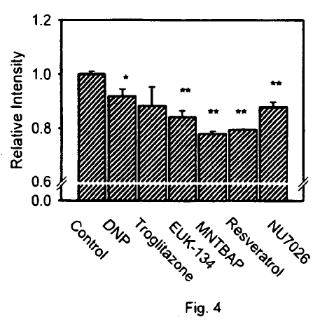

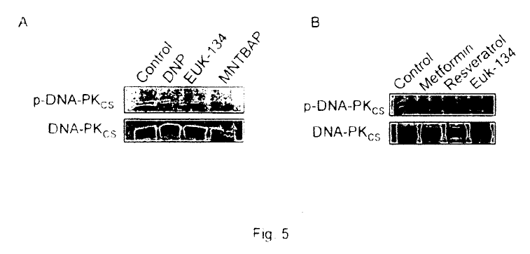

Reactive oxygen species production in MCF7 cells was significantly decreased after treatment with NU7026, DNP, TZD, Euk-134, MnTBAP and Resveratrol (FIG. 4). These results indicated that the DNA-PKcs inhibitor, NU7026, functions as an inhibitor of reactive oxygen species production. These results also suggested the possibility that DNA-PKcs may be suppressed by NU7026, DNP, TZD, Euk-134, MnTBAP and Resveratrol in vitro and this possibility was examined as described in the next Example.

### Example 5

### Suppression of DNA-PKcs with DNP, Euk-134, MnTBAP, Metformin and Resveratrol

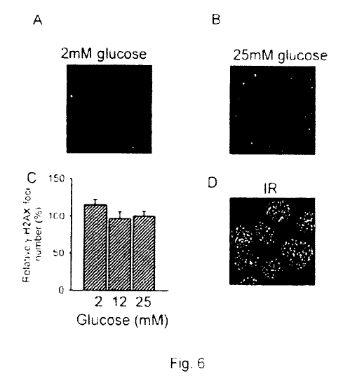

DNP, Euk-134, MnTBAP, metformin and resveratrol decreased DNA-PKcs activation in the presence of 25 mM glucose (FIG. 5). These results indicate that known scavengers of reactive oxygen species or metabolic uncouplers, Euk-134, MnTBAP, Resveratrol and DNP, are indeed DNA-PKcs inhibitors. As shown in Example 4 and FIG. 4, the DNA-PKcs inhibitor NU7026 is an inhibitor of reactive oxygen species production.

### Example 6

### DNA-PKcs Activation by Glucose is not Due to DNA Double-Stranded Break (DSB) Induction

To determine whether the activation of DNA-PKcs with increasing glucose concentrations (Example 3) is due to increased double-stranded breaks (DSB) in DNA, the number of nuclear foci that are double-positive for γ-H2AX was determined using available procedures (Rogakou et al., J. Biol. Chem. 273: 5858-68 (1998)) and immunostaining for p53 Binding Protein 1 (53BP1). Phosphorylated histone H2AX (“γ-H2AX”) recruits MDC1, 53BP1, and BRCA1 to chromatin near a double-strand break (DSB) and facilitates efficient repair of the break.

Frozen sections and cultured cells were fixed in 2% paraformaldehyde in PBS for 20 min, and washed in PBS. Cultured cells were permeabilized in cold 70% ethanol. Tissue sections were permeabilized with I% Triton X-100 for 5 min. The preparations were then stained with monoclonal mouse anti-γ-H2AX (Upstate BioTech, Lake Placid, N.Y.) and polyclonal rabbit anti-53BP1 (Novus Biologicals, Littleton, Colo.) primary antibodies. To detect these antibodies, Alexa-555-labeled goat anti-mouse and Alexa-488-labeled goat anti-rabbit secondary antibodies (Invitrogen, Eugene, Oreg.) were used as described by Rogakou et al. (1999). Cells were mounted in a Vectashield mounting medium with DAPI or propidium iodide (Vector, Burlingame, Calif.) staining. Microscopy was performed with a Nikon PCM 2000 (Nikon Inc, Augusta, Ga.). The foci were counted by eye from randomly chosen 100-250 cells of MCF-7 cells in a blinded fashion. Double-stranded breaks in DNA were visualized with immunofluorescent staining of γ-H2AX (red) and phospho-53BP1 (green) foci in cells exposed to 2 mM and 25 mM glucose or ionizing radiation.

Most foci were double-positive (yellow in original) for γ-H2AX and phospho-53BP1 (FIG. 6). The number of DNA double-stranded breaks did not change with increasing glucose (FIG. 6).

Phosphorylation of Ser15 in p53, which is induced by DNA double-stranded breaks, also did not change with glucose (data not shown). Thus, the energy-induced activation of DNA-PKcs does not occur in response to DNA double-stranded breaks, or at least in response to DNA double-stranded breaks that induce γ-H2AX foci.

### Example 7

### Calorie Restriction Induces Suppression of DNA-PKcs In Vivo

The results in Example 1 through Example 6 suggest that DNA-PKcs is regulated by nutrition and energy metabolism that is coupled to reactive oxygen species-production. To determine whether the basal activity of DNA-PKcs is regulated by nutrition in vivo, DNA-PKcs activity was examined in animals fed ad libitum or subjected to short-term calorie restriction.

It was not possible to quantify DNA-PKcs activity in calorie-restricted rodent tissues because the DNA-PKcs expression level in untreated rodent cells is very low, and there is no phospho-specific antibody capable of detecting activated rodent DNA-PKcs. Instead, DNA-PKcs activity was examined in biopsy samples from the soleus muscle of age- and sex-matched rhesus monkeys (Macaca mulatta) (18-25 years old, 54-75 years old in human age) fed ad libitum or short-term calorie-restricted (30% of ad libitum for 3.4 years prior to biopsy). Workers have generated data indicating that calorie-restriction decreases body weight, decreases the amounts of reactive oxygen species, and reduces serum glucose, therefore tests were performed to ascertain whether calorie-restriction would decrease the basal DNA-PKcs activity.

Animal care was provided in accordance with the NIH Guide for the Care and Use of Laboratory Animals and this research was approved by the. Institutional Animal Care and Use Committee of the Oregon National Primate Research Center. Female rhesus macaques (Macaca mulatta) were matched by body weight and age, then assigned to ad libitum control or calorically-restricted (CR) treatment groups. The monkeys were singly-caged indoors at a temperature of 24° C. under a fixed 12L:12D photoperiod, with unlimited access to drinking water. All animals received a specially formulated monkey chow that included additional vitamin and minerals to avoid any deficiencies in essential nutrients. Feedings were conducted at 0800 h and 1500 h each day, but with calorically-restricted animals receiving 30% fewer calories. The monkeys' diets were also supplemented with daily fresh fruits or vegetables. Age-matched (18-25 years of age, equivalent to 54-75 human years) animals fed ad libitum (n=5) or calorie-restricted (n=5) for 3.4 years were euthanized and a biopsy of soleus muscle was obtained.

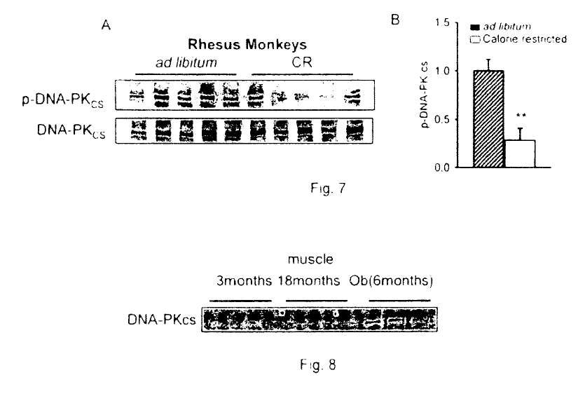

Calorie-restriction significantly suppressed the basal activity of DNA-PKcs (1.0±0.12 vs. 0.28±0.12 in arbitrary units, p=0.002) (FIG. 7A-B). Therefore, the basal activity of DNA-PKcs in vivo is also induced by energy-induced signals.

### Example 8

### Calorie Excess (Obesity) Increases DNA-PKcs Expression In Vivo

This Example addresses whether obesity is associated with alteration of DNA-PKcs and whether DNA-PKcs is causally linked to obesity and aberrant metabolic controls in obese state. In particular, DNA-PKcs levels were examined in the skeletal muscle, liver and white adipose tissue (WAT) of ob/ob mice (leptin-deficient mice, a genetic model of morbid obesity) compared with lean controls. Significant increases in the expression of DNA-PKcs was observed in these tissues from ob/ob mice (not all data shown). FIG. 8, for example, shows increased DNA-PKcs expression levels in skeletal muscle. These results indicate that the DNA-PKcs activity is increased in response to obesity.

### Example 9

### DNA-PKcs Suppresses Diet-Induced Obesity; SCID Mice are Resistant to Diet-Induced Obesity

Further experiments were conducted to address the potential role of DNA-PKcs in energy metabolism in vivo, the functional significance of the DNA-PKcs suppression in calorie-restricted primates (Example 7) and the increase in DNA-PKcs expression in ob/ob mice (Example 8). In particular, wild-type (WT, +/+) and SCID (SCID/SCID) littermates congenic (backcrossed at least 11 times) in a C57BL/6J background were fed regular rodent chow diet (RCD, 12% fat by calories, Zeigler, Rodent NIH-31), medium-fat diet (MFD, 22% fat by calories, Lab Diet) or high fat diet (HFD, 60% fat by calories, F3282, Bio-sery or D12492, Research Diets) after weaning (3 weeks of age) and monitored for 32 weeks.

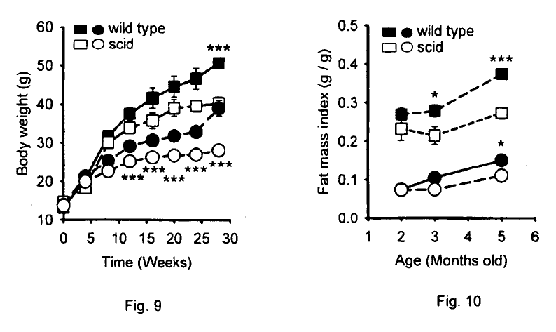

WT and SCID mice had similar mortality rates within the age-range studied here, although SCID mice did have a shorter mean lifespan than WT mice. Body weight was recorded with five-week intervals. WT and SCID mice had similar body weight at weaning and SCID mice gained slightly less weight than the WT littermates on a regular chow diet (data not shown). However, SCID mice gained significantly less weight than WT mice on a medium fat diet and high fat diet (FIG. 9).

### Example 10

### SCID Mice Gain Less Fat on a Medium- or High-Fat Diet

To determine the source of the body weight difference observed as described in Example 9, the fat and lean mass of the animals used in these experiments were measured using NMR spectroscopy (Bruker BioSpin Corporation, Houston, Tex.). Body fat indices were calculated by dividing fat or lean mass by body weight.

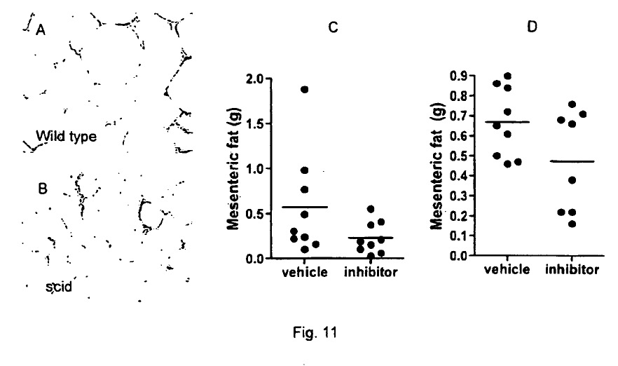

As shown in FIG. 10, SCID mice had lower fat mass index (gm of fat per gm of body weight) on the medium fat diet (MFD), but had similar lean mass (data not shown). Similar results were obtained for mice maintained on a high fat diet (HFD). In addition, fat tissues in SCID mice fed HFD for six months had significantly smaller mean fat cell size (FIGS. 11A and 11B).

Consistent with these results, treatment with DNA-PKcs inhibitor Cpd 36 (8 mg/kg body weight, twice daily by oral gavage for 3 months) reduced mesenteric fat significantly in mice maintained on a HFD diet (3 months) compared to the vehicle (control) treatment (FIGS. 11C and 11D).

In order to measure intestinal fat absorption, a synthetic diet containing 5% sucrose polybehenate, which is not absorbed, was fed to WT and SCID mice for 3 days. Fecal samples were collected and analyzed for fatty acid methyl esters by gas chromatography. Fat absorption was calculated from the ratio of behenic acid to the other fatty acids in the diet. Intestinal fat absorption was the same between WT and SCID mice (SCID, 98.2±0.22% and WT, 98.5±0.23%) indicating that malnutrition did not contribute to obesity resistance in SCID mice.

### Example 11

### SCID Mice have an Increased Metabolic Rate

Decreased body weight, in the absence of increased food intake, suggests that SCID mice have increased metabolic rate. Indeed, oxygen consumption and carbon dioxide production were elevated in 7 month old (data not shown) and 16 month old (Table 1) SCID mice compared to WT littermates.

TABLE 1
Basal metabolic rate and locomotor activity
of 16 month WT and SCID mice.

| | Wild-Type | | SCID | |
|---|---|---|---|---|
| | Day | Night | Day | Night |
| VO₂ | 3312 ± 125 | 3492 ± 112 | 3607 ± 126 | 3943 ± 181 |
| ml/kg/hr | | | (p = 0.12) | (p = 0.06) |
| VCO₂ | 2762 ± 123 | 3077 ± 123 | 3002 ± 160 | 3559 ± 202 |
| ml/kg/hr | | | (p = 0.25) | (p = 0.06) |
| Heat | 16.1 ± 0.7 | 17.2 ± 0.6 | 17.3 ± 0.6 | 19.4 ± 0.9 |
| cal/hr/g | | | (p = 0.21) | (p = 0.07) |
| X total | 279 ± 26 | 437 ± 38 | 206 ± 6 | 402 ± 22 |
| counts | | | (p = 0.02) | (p = 0.4) |
| (activity) | | | | |
| Y total | 229 ± 34 | 374 ± 49 | 192 ± 14 | 367 ± 29 |
| counts | | | (p = 0.34) | (p = 0.9) |
| (activity) | | | | |
| Z total | 122 ± 20 | 245 ± 31 | 86 ± 32 | 161 ± 38 |
| counts | | | (p = 0.35) | (p = 0.12) |
| (activity) | | | | |

Locomotor activity was also measured. Mice were studied for a period of 72 hr using the Comprehensive Laboratory Animal Monitoring System (CLAMS; Columbus Instruments, Columbus, Ohio). Food consumption was monitored by electronic scales, and movement, by X/Y/Z laser beam interruption. The level of locomotor activity of SCID mice (16 months old) was similar or slightly lower than WT mice (Table 1).

Consistent with the results described above, SCID mice on a high fat diet (HFD) had significantly lower serum leptin levels (HFD WT 57.8±20 ng/ml, HFD Scid 17.4±13 ng/ml, n=3-4, p<0.05). Also, visceral adiposity in HFD-fed SCID mice was dramatically reduced compared to WT littermates (data not shown).

### Example 12

### Obese, Middle-Aged SCID Mice Express Higher Levels of Thermogenic Genes in BAT(Brown Adipose Tissue)

Brown adipose tissue (BAT) is a highly thermogenic tissue and is important for protection against obesity (Lowell et al. Nature 366: 740-42 (1993)). Using real-time PCR, the expression of genes important for thermogenesis was examined in brown adipose tissue isolated from lean (L, 3 mo old), obese (Ob, induced by feeding HFD) and middle-aged (MA, 14 mo old) WT and SCID littermates. In particular, the expression of the following thermogenesis genes was examined: PGC-1α and PGC-1β (mitochondrial-biogenesis), UCP1 (mitochondrial uncoupling), ERRα (mitochondrial gene expression), and CPT1b and PPARδ (fatty acid oxidation).

Total RNA was isolated by the TRIzol method. Total RNA isolated from WT or SCID tissues was reverse transcribed with Taqman reverse transcription reagents. Reactions were performed in 96-well format using Taqman core reagents and a Prism 7900HT sequence detector (ABI). The RT-PCR was performed for 40 cycles at the following cycling condition: 5° C. for 10 min initial denaturation; then 40 cycles of 95° C. denaturation for 15 sec, 60° C. anneal/extension for 1 min for each cycle. 18S RNA was used as the internal standard for all mRNA.

Compared to lean WT mice, the expression of most of these genes, including PGC-1α, UCP1, CPT1b and PPARδ was dramatically reduced in middle-aged WT mice (FIG. 12). In contrast, the expression of PGC-1α did not decline in obese or middle aged SCID brown adipose tissue (BAT) and was significantly higher than that in corresponding BAT from WT littermates. Except for PGC-1β, the expression of these thermogenic genes were significantly higher in SCID BAT compared to that in BAT from obese and or middle-aged WT littermates. In SCID BAT, the amount of mitochondrial DNA increased more than 2.8-fold compared to WT BAT.

Increased expression of PGC-1α, PPARδ and UCP3 mRNA would indicate that SCID mice have a higher rate of fat burning. Indeed, the serum levels of 8-hydroxybutyrate, a by-product of fat oxidation, was higher in SCID mice after fasting (HFD WT 384±27 μM, HFD Scid 428±33 μM, N=3-4, p<0.05).

Brown adipose tissue (BAT) maintains body temperature by non-shivering thermogenesis, especially during fasting-like conditions such as hibernation. To examine whether the increased expression of the thermogenic genes in SCID BAT affected body temperature regulation, the core body temperature in lean, obese and middle-aged mice was measured before and after overnight fasting.

Measurements of body temperatures of WT and SCID mice indicated that these mice generally had similar temperatures. The body temperature decreased for both WT and SCID mice after overnight fasting to conserve energy (data not shown). Consistent with the expression levels of the thermogenic genes, the fasting body temperatures of lean WT and SCID mice were the same (data not shown). However, the fasting body temperatures of obese as well as middle-aged SCID mice were approximately 0.5-1° C. higher than that of the corresponding obese and middle-aged WT mice.

### Example 13

### Obese, Middle-Aged SCID Mice Express Higher Levels of Thermogenic Genes in WAT (White Adipose Tissue)

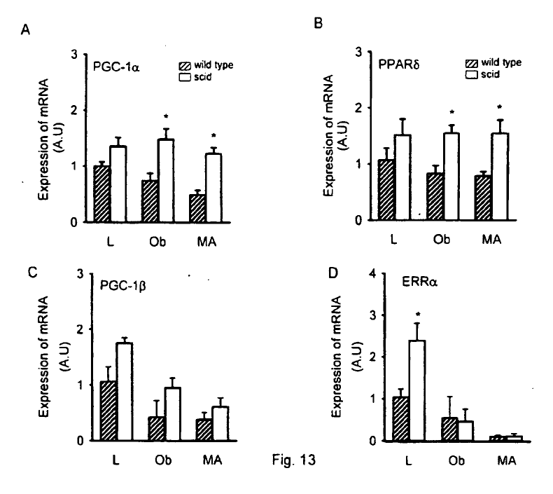

The expression levels of the thermogenic genes were measured in white adipose tissues. As shown in FIG. 13 the expression of thermogenic genes was also increased in obese and middle-aged SCID white adipose tissues compared to WT white adipose tissue.

### Example 14

### SCID Muscle has Higher Expression Levels of Genes Important for Mitochondrial Biogenesis and Function

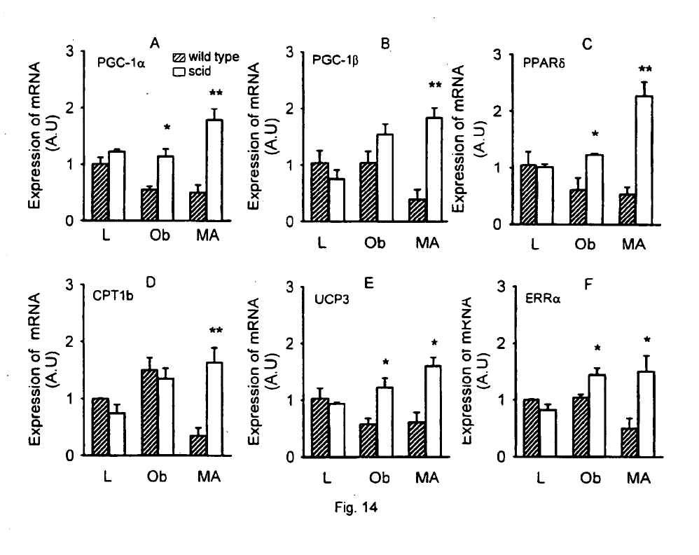

The increased expression of the genes involved in energy production and energy expenditure in brown and white adipose tissue prompted further experiments to measure the expression of these genes in skeletal muscle. Compared to lean WT mouse skeletal muscle, the expression of PGC-1α, PPARδ and UCP3 was reduced in the skeletal muscle of obese and middle-aged WT mice (FIG. 14). Moreover, the expression of CPT-1b, ERRα and PGC-1β was reduced in the skeletal muscle of middle-aged WT mice relative to that observed in lean WT mice (FIG. 14). These results are consistent with previous reports of the obesity-related decline in humans and rodents (Kelley et al. Diabetes 51: 2944-50 (2002); Sparks et al. Diabetes 54: 1926-33 (2005)) and the age-related decline in humans and rodents (Petersen et al., Science 300: 1140-42 (2003); Short et al. Proc. Natl. Acad. Sci. USA 102: 5618-23 (2005); Ling et al. J. Clin. Invest. 114: 1518-26 (2004).

The expression of these genes in SCID skeletal muscle had an inverse pattern compared to that in WT skeletal muscle. The expression levels of these genes were also significantly higher in obese and middle-aged SCID muscle compared to WT muscle, except for the CPT-1b gene, which was higher only in middle-aged SCID muscle. In contrast to what was seen in WT mice, the expression levels of these genes were significantly increased in middle-aged SCID muscle compared to lean SCID muscle (p=0.06, 0.007, 0.007, 0.04, 0.01 and 0.1 for PGC-1α, PGC-1β, PPARδ, CPT1b, UCP3 and ERRα, respectively).

### Example 15

### DNA-PKcs Promotes Mitochondrial Decline and SCID Muscle Contains More Mitochondrial DNA (mtDNA)

Quantitative Real-Time PCR was used to assess the relative amounts of nuclear DNA and mtDNA, to permit assessment of the ratio of mtDNA to nucleic DNA, which reflects the tissue concentration of mitochondria per cell. Muscle tissues were homogenized and digested with Proteinase K overnight in a lysis buffer for DNA extraction by conventional phenol-chloroform method. Quantitative PCR was performed using the following primers:

mtDNA Specific PCR Primers:

| | | |
|---|---|---|
| forward | 5′-CCGCAAGGGAAAGATGAAAGA-3′ | (SEQ ID NO: 5) |
| reverse | 5′-TCGTTTGGTTTCGGGGTTTC-3′ | (SEQ ID NO: 6) |

Nuclear Specific PCR Primers:

| | |
|---|---|
| forward | |
| 5′-GCCAGCCTCTCCTGATTTTAGTGT-3′ | (SEQ ID NO: 7) |
| reverse | |
| 5′-GGGAACACAAAAGACCTCTTCTGG-3′ | (SE ID NO: 8) |

An SYBR Green PCR kit was used with a Prism 7900HT sequence detector (ABI) using a program of 20 minutes at 95° C., followed by 50 to 60 cycles of 15 seconds at 95° C., 20 seconds at 58° C. and 20 seconds at 72° C.

Consistent with the PGC-la mRNA expression pattern, there was more mitochondrial DNA in SCID muscle compared to WT muscle in middle-aged mice (FIG. 15).

SCID skeletal muscle contains more mitochondria. Mitochondria in skeletal muscles were visualized with transmission electron microscopy. The samples were fixed for 1 h in a mixture of 2.5% glutaraldehyde, 4% paraformaldehyde, in phosphate buffer (pH 7.4), washed in distilled water, and placed in 1% osmium for 1 hour. The samples were then washed again and dehydrated with acetone before infiltration and embedding with EPON 812. The EPON-embedded samples were baked at 60° C. for 48 h. Ultrathin sections (about 60-90 nm) were cut on a Leica Ultracut ultramicrotome, picked up on to copper grids stained with uranylacetate and lead citrate, and examined in a JEOL 1200EX Transmission Electron Microscope (JEOL).

The muscle from middle-aged WT mice had smaller and fewer mitochondria compared to the skeletal muscle from middle-aged SCID littermates (data not shown). While the mitochondrial volume in skeletal muscle of WT and younger SCID mice (3 month-old) was similar (data not shown), morphometric analyses indicate that older SCID (14 month-old) skeletal muscle had approximately 30% greater mitochondrial volume than WT skeletal muscle.

### Example 16

### SCID Mice Have Exceptional Running Endurance

Previous studies have shown that increased expression of PPARδ or PGC-1α in skeletal muscle decreases exercise-induced fatigue. Therefore, tests were performed to ascertain what was the physical endurance of 4, 7 and 14 month-old SCID and WT mice using treadmill running. Prior to the exercise test, the mice were accustomed to and trained by running on an Exer-3/6 mouse treadmill (Columbus Instruments) at 7 m/min for 5 minutes for 3 days. For the endurance test, the treadmill speed was increased to 10 m/min and mice were allowed to run until exhaustion.

While SCID and WT mice ran similar distances at 4 months of age, SCID mice ran almost twice the distance of WT mice at 7 and 14 months of age (FIG. 16). Physical fitness is comprised not only of endurance but the ability to recover from exercise. To test their ability to recover, the obese and middle-aged mice were subjected to treadmill running for three consecutive days (FIG. 17). Lean SCID and WT mice ran similar distances on the first day but the lean SCID mice ran almost twice the distance on day 3. Since lean SCID mice and WT mice had similar body weights, the reduced endurance of WT mice was not caused by excess mass. Obese SCID mice ran greater distances than WT mice from day 1 and this difference increased with each successive day of running. By day 3, obese SCID mice were able to run twice the distance of obese' WT mice. The distance run by middle-aged SCID mice was almost twice that of WT mice from day 1 and this trend continued to increase with each successive day of exercise. By day 3, middle-aged SCID mice ran 2.5 to 3 times the distance of WT mice.

### Example 17

### DNA-PKcs Suppresses AMPK Signaling and SCID Mice Have a Higher Basal AMPK Activity

In BAT and skeletal muscle, PGC-1α expression and mitochondrial biogenesis are induced under conditions of metabolic demand such as calorie restriction (Nisoli et al. Science 310: 314-17 (2005)), cold exposure (Puigserver et al. Cell 92 : 829-39 (1998)) and endurance exercise training (Wu et al. Science 296: 349-52 (2002)). Expression of PGC-1α and mitochondrial biogenesis are induced by 5′-AMP-dependent protein kinase (AMPK) (Hardie & Carling, Eur. J. Biochem. 246: 259-73 (1997)), which is activated by energy depletion (Zong et al. Proc. Natl. Acad. Sci. USA 99: 15983-87 (2002)), and requires LKB1. This information suggests that DNA-PKcs deficiency may cause AMPK activation, which in turn results in enhanced PGC-1α expression (Example 14), mitochondrial biogenesis and function (Example 15) and physical fitness (Example 16).

Alternatively, because exercise increases AMPK activity and PGC-1α expression, it is also possible that the increased PGC-1α expression and the metabolic phenotypes seen in SCID mice are due to increased muscle activity of SCID mice rather than deficiency of DNA-PKcs per se. As shown in Table 1, the level of locomotor activity of SCID mice, as measured by beam breaks, was similar to or slightly lower than WT mice. Voluntary wheel running activity was also similar between SCID and WT mice (not shown). Therefore, these results rule out the possibility that increased muscle activity is the cause for the metabolic phenotype seen in SCID mice.

AMPK activity was then examined in tissues isolated from resting middle-aged mice (FIG. 18). The basal activity of AMPK in skeletal muscle, white adipose tissue (WAT) and liver was visualized by immunoblotting with antibody specific for phospho-Thr172 (Hawley et al., J. Biol. 2: 28 (2003); Woods et al. Curr. Biol. 13: 2004-8 (2003)), a surrogate marker for AMPK activation.

The basal activity of AMPK was higher in SCID mice than WT mice in muscle and WAT, but not in liver (FIG. 18). Consistent with this, AMPK phosphorylation of the Ser79 in acetyl-CoA carboxylase (ACC), which inactivates ACC and thereby stimulates fatty acid oxidation, tended to be higher in SCID WAT and skeletal muscle (FIG. 18; see also, Munday et al., Eur. J. Biochem. 175: 331-38 (1988); Sim et al, FEBS Lett. 233: 294-98 (1988)).

As expected from the results that the basal activity of AMPK in liver was similar both in SCID and wild type mice (FIG. 18), the hepatic expression of PGC-1α and PGC-1β mRNA was also similar in SCID and WT mice (data not shown). These results indicate that AMPK activity is higher in SCID tissues.

### Example 18

### DNA-PKcs Inhibitor Activates AMPK

Although AMPK activity is higher in SCID tissues (Example 17), it is possible that potential confounding variables, rather than deficiency of DNA-PKcs per se, may have induced AMPK activity. To determine if the activity of AMPK was higher in SCID tissues because of energy depletion, ATP and ADP levels were measured in skeletal muscle of middle-aged mice.

The level of ATP and the ADP/ATP ratio in cells were determined using an

ENLITEN ATP assay kit (Promega) and ApoGlow assay kit (Cambrex), respectively. As shown in FIG. 19, SCID muscle tended to have slightly higher “energy charge” (more ATP, lower ratio of ADP/ATP) than WT muscle. To determine whether the AMPK-inhibitory function of DNA-PKcs is cell autonomous, quiescent MCF7 cells were treated with 1 and 2.5 μM NU7026. In this concentration range, NU7026 is a highly selective inhibitor of DNA-PKcs (IC₅₀=0.23 μM). IC₅₀ values for NU7026 for related kinases such as PI3K and ATM are 13 μM and >100 μM, respectively.

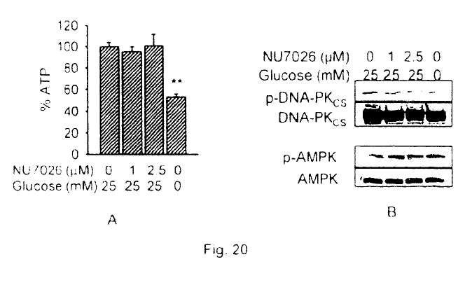

Treatment of MCF7 cells with NU-7026 activated AMPK without significantly affecting ATP levels, indicating that inhibition of DNA-PKcs did not cause AMPK activation by depleting energy (FIG. 20). NU7026 treatment also activated AMPK in differentiated C2C12 myoblasts, a model for skeletal muscle cells (data not shown).

### Example 19

### DNA-PKcs RNAi Activates AMPK

AMPK activation by DNA-PKcs inhibition was further demonstrated in an adipocyte differentiation system using small interfering RNA (siRNA). Mouse 3T3-L1 preadipocytes were purchased from the ATCC. Cells were passaged before confluence and used before 10th passage. 2 μM of DNA-PK RNAi (Dharmacon) or Scrambled RNAi (Dharmacon) were transfected into 3T3-L1 cells using Lipofectamine 2000 (Invitrogen). After 18 h, the cells were differentiated into adipocytes by treatment of postcontluent cells with 10% FBS, 1 μg/mL insulin, 1 μM dexamethasone (DEX), and 0.5 mM isobutyl-1-methylzanthine (MIX). The differentiation medium was withdrawn 2 days later and replaced with medium supplemented with 10% FBS and 1 μg/mL insulin. After 2 days in insulin-containing medium, the cells were then cultured in DMEM containing 10% FBS for 2 days before analysis.

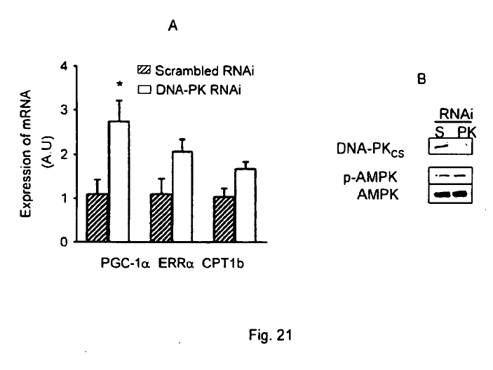

Knocking-down DNA-PKcs production in 3T3-L1 differentiated adipocytes with RNAi specific for DNA-PKcs increased AMPK activity and expression of PGC-1α, ERRα and CPT1b mRNA (FIG. 21). Taken together, these results indicate that DNA-PKcs is a tonic inhibitor of AMPK activity.

### Example 20

### SCID Mice are Insulin-Sensitive

Activation of AMPK promotes glucose uptake in skeletal muscle and increases insulin sensitivity and PGC-1α-dependent signaling is suppressed in the skeletal muscles of diabetics. In view of these results, tests were performed to ascertain whether SCID mice are more insulin sensitive.

In the obese (Ob) high-fat diet and middle-aged (MA) groups, SCID mice had similar fasting glucose levels as WT littermates. However, measurement of plasma insulin concentrations before and after intraperitoneal injection of glucose in overnight fasted mice (n=8-12), indicated that SCID insulin levels were significantly lower than observed in WT littermates, indicating that SCID mice are more insulin sensitive (FIG. 22).

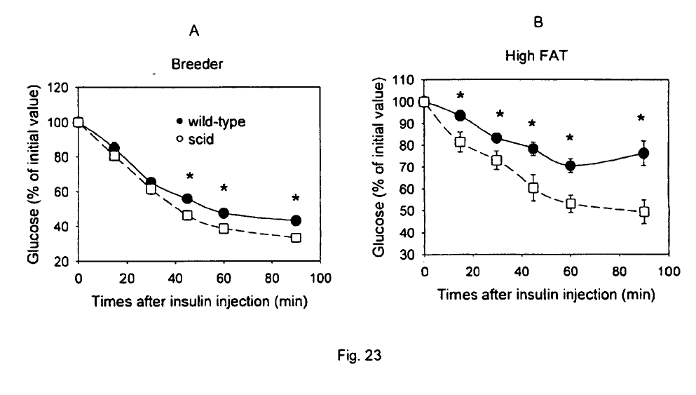

Consistent with this notion, insulin injection reduced serum glucose levels dramatically faster in SCID mice compared to WT mice (FIG. 23).

### Example 20-1

### SCID Metabolic Phenotype is not Lymphocyte-Related

To further demonstrate that the metabolic phenotype in SCID mice shown in all previous Examples is not related to immunodeficiency, the metabolic properties of mice deficient in Rag1 were investigated. Rag1 is a nuclease that is essential for V(D)J recombination and lymphocyte development (Oettinger et al., Science 248: 1517-23 (1990)). Because Rag1 and DNA-PKcs participate sequentially in the same VDJ recombination pathway in lymphocytes, the immunological phenotype and immune status of Rag1^{-/-} and SCID mice are very similar (Mombaerts et al. Cell 68: 869-77 (1992). Moreover, because Rag1 is primarily expressed in lymphocytes, Rag1^{-/-} mice should only exhibit the phenotype attributable to lymphocyte depletion.

To investigate whether lymphocyte depletion by itself induced a metabolic phenotype, Rag1^{-/-} mice (congenic in C57BL/6J background) were fed a MFD (medium fat diet) and the body weights of these mice were measured. Unlike SCID mice, Rag1^{-/-} mice had the same body weight as WT mice (data not shown). Also, there was no significant difference in the fasting body temperatures (data not shown) between lean or obese Rag1^{-/-} and WT mice. Consistent with these findings, there was no difference in the expression level of PGC-1a and PPARd mRNA between Rag1^{-/-} and WT tissues (data not shown). There was also no difference in physical fitness because lean and obese Rag1^{-/-} mice ran similar distances as the WT controls before exhaustion (data not shown). Taken together, these findings indicate that the metabolic phenotype of SCID mice is unrelated to its immune status.

### Example 21

### AKT Activity is Higher in Insulin-Sensitive Tissues of SCID Mice

AKT, the effector kinase for insulin and growth factor signaling, is important for cellular survival. It also plays a critical role in insulin-stimulated vasodilation and glucose uptake. In insulin resistant states, AKT activity is diminished, reducing glucose uptake by muscle and causing hypertension. Other workers have published that DNA-PKcs activates AKT by causing phosphorylation of Ser 473, however, this assertion conflicted with data obtained by the inventors that SCID mice had increased insulin sensitivity. Subsequently, the hypothesis that PKcs activates AKT by causing phosphorylation of Ser 473 was disproven by Sarbassov et. al. who showed that it is the Rictor complex that phosphorylates Ser 473 of AKT, not DNA-PKcs (Sarbassov et al., Science 307: 1098-1101 (2005)).

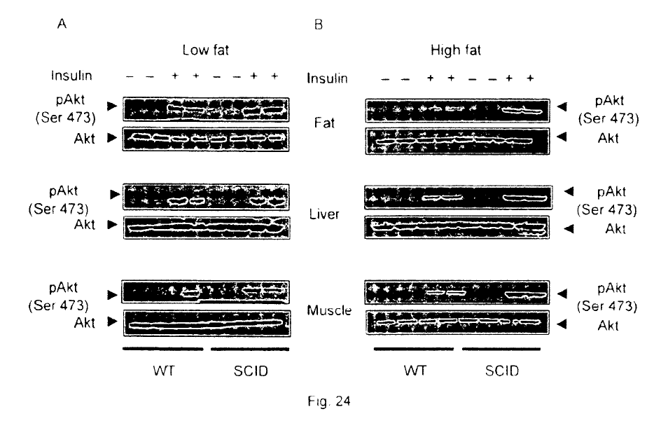

To further clarify this issue, tests were conducted to ascertain whether DNA-PKcs plays a role in inhibiting AKT. In particular, immunoblots were probed with antibody specific for AKT phospho-Ser 473. Injection of insulin significantly increased AKT activity in muscle, fat and liver, and this effect was greater in SCID tissues than was observed in WT tissues (FIG. 24). Enhanced Akt activation was also observed in DNA-PK −/− (null) MEFs after insulin treatment (data not shown). Moreover, the difference between SCID and WT AKT activation upon insulin injection was greater the animals were fed a high fat diet (HFD; FIG. 24). Injection of insulin also increased insulin receptor tyrosine phosphorylation to a greater extent in SCID muscle, in particular, after high fat diet treatment (data not shown). In addition, insulin injection dramatically reduced 1RS1 phosphorylation at Ser 307 and 636/639 in Scid muscle after maintenance on a HFD (data not shown).

### Example 22

### SCID Mice Show Elevated Levels of Sirt1, eNOS and VEGF

Results in the previous Examples regarding the metabolic phenotype of SCID mice indicate that DNA-PKcs deficiency mimics calorie restriction (CR). Caloric restriction also increases Sirt1 protein levels and the expression of genes that increase mitochondrial biogenesis and oxidative phosphorylation such as PGC-1α. It has been shown that these metabolic effects of caloric restriction require eNOS (endothelial nitric oxide synthase), which is increased during caloric restriction. Because caloric restriction suppresses DNA-PKcs activity (Example 7) and calorie excess increases DNA-PKcs expression (Example 8), it is possible that the metabolic effects of caloric restriction may be mediated by a caloric restriction-induced decrease in DNA-PKcs activity. The impact of DNA-PKcs deficiency upon expression of other genes was therefore examined further.

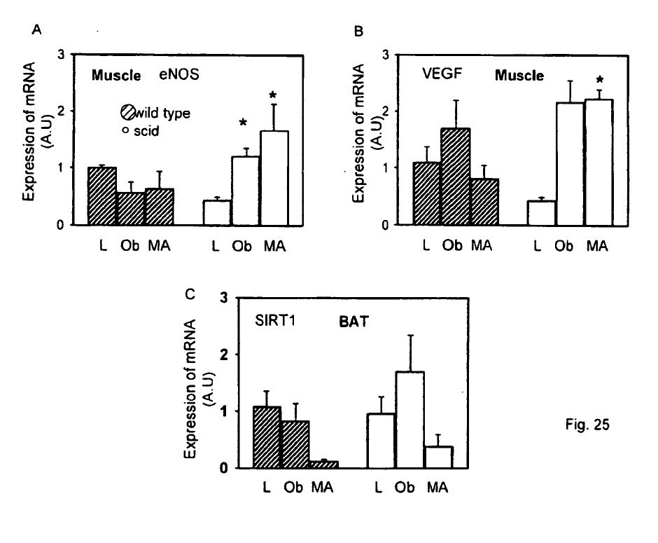

SCID tissues have elevated levels of Sirt1 protein and eNOS expression (FIG. 25). Together with the results shown in Example 12, 13 and 14 for the elevated levels of PGC-1α and the other thermogenic genes in SCID tissues, these results support the hypothesis that the metabolic effects of caloric restriction may be mediated by caloric restriction-induced suppression of DNA-PKcs activity.

eNOS, which produces nitric oxide (NO), mediates vasodilation and decreases blood pressure. eNOS expression is activated by VEGF (vascular endothelial growth factor), a growth factor that mediates angiogenesis and blood vessel formation. To determine whether the increased expression of eNOS is related to altered expression of VEGF in SCID mice, we measured VEGF mRNA in SCID tissues. VEGF expression was significantly elevated in SCID muscle compared to WT muscle (FIG. 25) suggesting that suppression of DNA-PKcs may increase blood vessel formation.

Blood pressure was measured using tail-cuff non-invasive blood pressure measurement methods. Consistent with the results above, SCID mice have decreased blood pressure (WT 100±8 mmHg, Scid 84±8 mmHg).

eNOS stimulates the expression of BDNF (brain derived neurotrophic factor), which is important for neurogenesis after stroke, long-term memory formation and suppression of anxiety and depression. Because SCID mice have increased eNOS expression, BDNF levels in the brain were measured and it was determined that the SCID brain has higher BDNF levels (>1.6 fold increase compared to the WT brain). These results indicate that DNA-PKcs may affect brain function.

### Example 23

### SCID Fat Tissues Exhibit Less Macrophage Infiltration

Inflammatory signaling not only has a pro-aging effect but also has metabolic effects. Obesity is accompanied by a marked increase in macrophage infiltration of white adipose tissue (WAT) and obesity is strongly associated with an increase in circulating levels of acute phase proteins and cytokines, which mainly originate from WAT (Xu H et al. J Clin Invest 112:1821 (2003); Weisberg SP et al. J Clin Invest 112:1796, 2003; Trayhurn P Br J Nutr 92:347, 2004; Stienstra R et al. Endocrin 2007). It is widely believed that obesity-induced insulin resistance and diabetes is mediated through macrophages that are recruited to fat cells and are activated by the stress signals emanating from overloaded fat. Since SCID mice are more insulin sensitive compared to WT mice (Example 20 and Example 21), the inflammatory signals in the adipose tissue of obese SCID and WT mice were investigated.

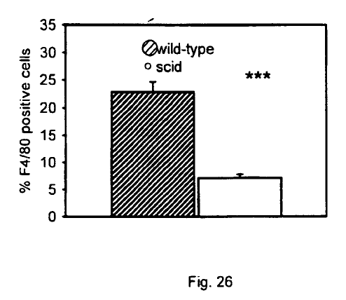

As shown in Example 10 (FIG. 11), the fat cell size in SCID mice is significantly smaller than that of the WT mice. As shown in FIG. 26, SCID fat tissue also contained fewer macrophages detectable with macrophage-specific F4/80 antigen (a marker for mature macrophages, Leenen P J et al. J Immunol Methods 174:5, 1994) than those in WT mice, indicating decreased inflammatory cell recruitment in the SCID fat tissues. These data indicate that DNA-PKcs promotes obesity-induced macrophage infiltration in white adipose tissues.

### Example 24

### The Loss of DNA-PKcs Function has an Anti-Inflammatory Effect

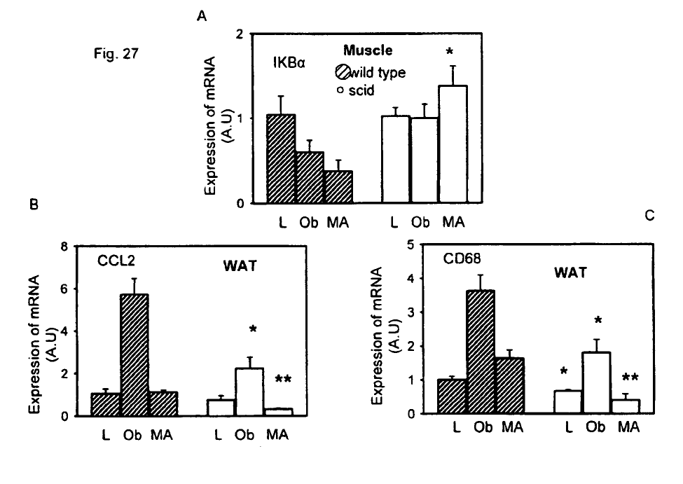

As shown in FIG. 27, SCID muscle in middle-aged animals expressed more IκBα, the inhibitor of the NFκB inflammatory pathway, and SCID white adipose tissues (WAT) expressed less CCL2 and CD68.

The chemokine CCL2, also known as monocyte chemoattractant protein-1, is a major factor driving leukocyte infiltration into tissues in a variety of inflammatory conditions. CCL2 (MCP-1) plays a major role in regulating immune/inflammatory responses, ischemic/reperfusion conditions and vascular permeability. CD68 (a 110-Kd transmembrane protein, a member of hematopoietic mucin-like molecule) is highly expressed by monocytes and tissue macrophages and used as a marker of inflammation (Holness C L et al. Blood 81:1607 (1993)). CCL2 and CD68 were not induced or only slightly increased in high fat diet-fed SCID (obese SCID) WAT, while these were increased in obese WT WAT. Thus, the loss of DNA-PKcs function has an anti-inflammatory effect on inflammatory gene expression in WAT.

### Example 25

### SCID Mice Have Less Anxiety and Depression

To evaluate whether SCID mice may also have altered anxiety, fear and depression-like traits, an elevated plus maze test was performed, which is a commonly used test to quantify the level of anxiety- and depression-like traits in rodents (Pellow et al. J. Neurosci. Methods 14: 149-67 (1985)). The elevated plus maze has narrow runways that are located 16 inches above the surface that are either closed or open. The animal is placed in the center of an elevated 4-arm maze in which 2 arms are open and 2 are enclosed. Mice generally avoid the open arms because of their fear of open space and height. The elevated plus-maze is used to determine the rodent's response to a potentially dangerous environment and anxiety-related behavior is measured by the degree to which the rodent avoids the unenclosed arms of the maze. Increase in the number of times the animal enters the open arm and the amount of time spent in the open arms reflect decreased anxiety and depression.

As shown in FIG. 28, almost all wild-type mice stayed in the closed arms of the plus maze to the exclusion of the open arms. In contrast, most SCID mice spent a significant portion of their time in the open arms (FIG. 28). One SCID mouse spent all of its time in the open arms (FIG. 28).

WT and SCID mice were tested in another anxiety test using the light-dark box (Bourin and Hascoet, Eur J Pharmacol 463:55 (2003)). The light-dark box test is based on the innate aversion of rodents to brightly illuminated areas and on the spontaneous exploratory behavior of rodents in response to a mildly stressful situation, that is, a novel environment and light (Crawley and Goodwin Pharmacol Biochem Behav 13:167, 1980). In the light-dark test, increased activity in the light compartment indicates decreased anxiety.

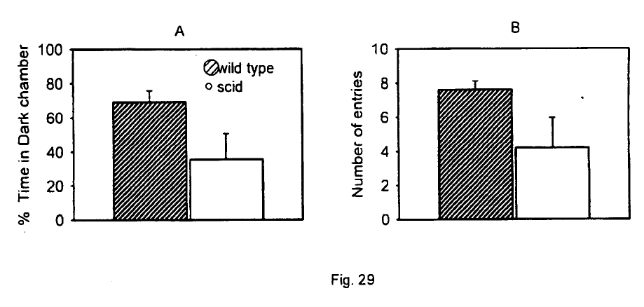

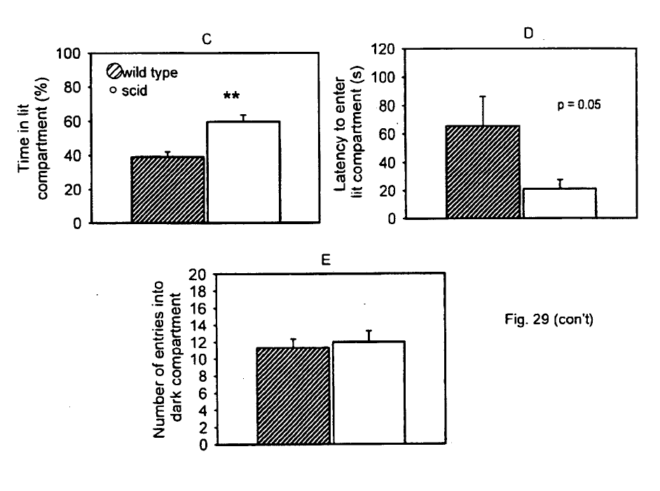

Consistent with the tendencies shown in the elevated plus maze test (FIG. 28), during the light-dark box test, SCID mice spent less time in the dark chamber and more time in the light compartment, although the number of entries into the dark compartment was the same in both SCID and wild type mice (FIG. 29). These results indicate that SCID mice have fewer anxiety-depression-like traits.

No evidence exists indicating that the fewer anxiety-depression-like traits in SCID mice are lymphocyte-related, and Rag1^{-/-} mice and WT mice did not show any difference in the elevated plus-maze test (data not shown). Accordingly, the non-lymphocyte-related aspects of the SCID phenotype are likely responsible for any reduced anxiety and depression observed in SCID mice.

### Example 26

### SCID Mice Have Greater Tolerance to Pain

Epidemiological studies indicate that people with mood disorders are twice as likely to have chronic pain compared to people without mood disorders (Ohayon et al., Arch. Gen. Psychiatry 60: 39-47 (2003)). Although it is possible that pain contributes to the depressed mood, it is also possible that perception of pain is increased in people with mood disorders. To determine whether SCID mice have altered pain tolerance, the latency for pain reaction to a hot plate was measured. The latency for SCID mice was significantly longer than WT mice at 52° C. (FIG. 30) and at 55° C. (data not shown), suggesting that the absence of DNA-PKcs may confer greater tolerance to pain (FIG. 30).

### Example 27

### SCID Mice are Resistant to Stress-Induced Binge Eating

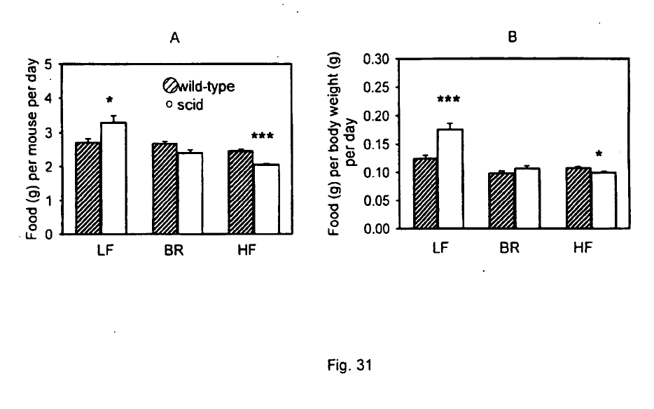

Mood disorders are often associated with eating disorders. Therefore, experiments were conducted to ascertain whether SCID mice may also be resistant to eating disorders. When group-housed with their littermates (4-5 per cage), 2-3 month-old SCID mice consumed greater amounts of the low fat diet (LFD) than the wild-type mice (FIG. 31) but same amount of medium fat diet (breeder diet, BR). High fat diet (HFD) consumption for SCID mice was slightly less than that of the wild-type mice. Furthermore, Scid mice maintained on a high-fat diet (>6 months) showed >50% reduction in both high-fat food consumption/mouse/day and high-fat food consumption/body weight/day compared to the WT mice (data not shown).

Previously group-housed mice were then isolated into individual cages so that only one mouse was present per cage. These isolated mice were then fed a low fat diet (LFD), a medium fat diet (MFD; breeder diet, BR) or a high fat diet (HFD) and the food intake of each mouse was measured. For these studies, green colored HFD was used so that the food consumption could be monitored and any uneaten food that was spilled or hoarded in the cage could be identified. Less than 10% of HFD appeared to have been spilled or hoarded.

When mice that had previously been group housed were socially isolated (one per cage), WT mice consumed a surprisingly large quantity of HFD, approximately three fold more than when they were group-housed, but SCID mice consumed the same quantity of HFD as when they had been group-housed (data not shown). Consumption of low fat (LF) and medium fat (BR) diets did not change upon isolation of SCID and wild-type mice (data not shown).

In another experiment, previously group-housed mice were isolated (one per cage) and fed a high fat diet for 5 days (day 0-day 5 after isolation), then a medium fat diet for the next 5 days (Breeder diet, day 5-day 10) and finally returned to a high fat diet for the following 10 days (day 10-day 20) (FIG. 32). Only WT mice were prone to stress (isolation)-induced binge eating of the high fat diet. For SCID mice, consumption of high fat diet was similar to that of the medium fat diet (breeder diet). Unlike the results obtained for isolated mice fed a high fat diet, consumption of medium fat diet (breeder diet) did not change upon isolation of previously group-housed SCID and WT mice.

Thus, the combination of a high fat diet and isolation elicited binge-eating behavior in wild-type mice but not in SCID mice.

The observed high fat diet-specific binge eating is very similar to human binge eaters, who also increase intake of fat rather than carbohydrates (Goldfein et al. Int. J. Eat Disord. 14: 427-31 (1993); Yanovski et al., Am. J. Clin. Nutr. 56: 975-80 (1992)). Both humans and rodents titrate the quantity of food consumed according to the caloric content of the food; this response serves to maintain caloric balance. Indeed, SCID mice were able to titrate food intake according to the caloric density of the food (i.e. less high fat diet and more medium fat diet), but wild-type mice showed a reverse-titration pattern (i.e. more high fat diet and less medium fat diet) during binge-eating. The results shown in this Example suggest that DNA-PKcs inhibitors/antagonists may be useful for treating eating disorders such as anorexia nervosa, bulimia and stress-induced binge eating.

### Example 28

### Decreased Mood/Stress Sensitivity and Pain Response in SCID Mice are Related to the Serotonergic Pathways

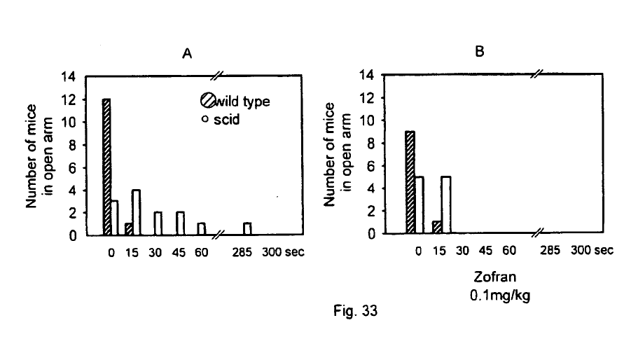

To test whether the decreased anxiety- and depression-like traits in SCID mice (Examples 25-27) are related to the serotonergic pathways, the elevated plus maze test was performed after intraperitoneal injection of serotonin antagonist GR38032F (Zofran) (Kilpatrick et al. Nature 330: 746-48 (1987)). The number of SCID mice that stayed in the open arm 30 sec or longer decreased dramatically after GR38032F injection (FIG. 33).

Because tolerance to pain (Example 26) is also linked to the serotonergic pathways, measurements of the latency to pain reaction on hot plate after saline, serotonin reuptake inhibitor fluoxetine (Prozac) or serotonin antagonist GR38032F (Zofran) injection were made. Fluoxetine injection increased the latency for pain reaction in wild-type mice but not in SCID mice, abolishing the difference in the latency for pain reaction between them (data not shown). Conversely, GR38032F (Zofran) injection decreased the latency for pain reaction in SCID mice but not wild-type mice, which also abolished the difference in the latency for pain reaction between them (data not shown). Taken together, these results suggest that mood, stress sensitivity and pain response are affected by DNA-PKcs activity, and suppression of DNA-PKcs may lead to decreased sensitivity to anxiety, depression or pain through the mechanisms that are linked to serotonergic pathways.

### Example 29

### DNA-PKcs Deficiency Confers Improved Memory

BDNF promotes long-term memory formation by causing phosphorylation of CREB, a transcription factor. Immunoblotting of brain samples indicated that CREB phosphorylation is increased in SCID brain (hippocampus) compared to WT brain (data not shown). These findings prompted further measurements of the cognitive ability of SCID and WT mice. In particular, two tests commonly used quantify memory: Morris water maze test and novel-object recognition test were performed.

The Morris water maze (MWM) test was performed following published procedures (Janus C et al. Neurobiol Aging 21:541, 2000; Zhang L et al. Behaviour Brain Res 173:246, 2006). The Morris water maze consists of a circular pool (4 ft. diameter, 30 in. high, San Diego Instruments) tilled with water kept at 25° C., and opacified with non-toxic latex paint. The water is changed weekly and given at least 24 hours to equilibrate to room temperature. A small square Plexiglas escape platform was placed at a fixed position in the centre of one quadrant and was hidden 1 cm beneath the water surface. The acquisition or training phase consists of eight training days (trial blocks) with four trials per day, starting at four different positions in a semi random order with a 15-min inter-trial interval. If an animal did not reach the platform within 120 s, it was be placed on the platform where it had to remain for 15 s before being returned to its home cage. Mice were dried off with a towel after each swim. Animals' trajectories were recorded using a computerized video-tracking system (Chromotrack, San Diego Instruments, USA) measuring path length and escape latency during each trial. The maze was surrounded by a number of fixed extra maze cues and, in addition, the experimental room was kept invariable. Spatial acuity was expressed as the percentage of time spent in each of the four quadrants of the pool and the number of times the mice crossed the former platform location.

Another memory test is the novel objection recognition test. To perform the novel object recognition test, two toys that were different in shape and color were placed in a cage (40 cm×40 cm×30 cm). Mice learned about the two toys for 5 minutes on five separate occasions (5×5 min/5 min, total 25 minutes of training) or for 25 minutes once (1×25 min). At varying times after the initial learning period (3 min, 3 h and 24 h), mice were returned to the cage with the two toys, except that one of the toys had been switched with a new toy. The amount of time the mice spent exploring the new toy was compared to the amount of time the mice spent exploring the original toy. This exploratory activity was monitored with video camera for 10 minutes. This novel objective recognition test is therefore based upon the premise that if the mice remembered the original toy, they spent more time exploring the new toy compared to the old toy.

In both of these memory tests, SCID mice performed significantly better than WT mice at ages 7 months and 12-14 months (FIG. 34). More surprisingly, the middle-aged (12-14 months old) SCID mice performed better in the Morris water maze than the young (7 months old) SCID mice. There was no significant age-related change in Morris water maze performance in WT mice.

Learning in the Morris Water Maze relies on swimming abilities and may be confounded by the genuine swimming abilities of SCID mice. Therefore, the spatial learning and memory of 7-month old and 14-month old SCID mice and wild type mice was evaluated by examining spatial memory retention. Mice were given learning trials at the beginning (0 week) and after 2 weeks and 4 weeks, the mice were retested in order to examine the spatial memory retention. SCID mice showed a significantly shorter latency in the Morris Water Maze test (FIG. 34).

FIG. 35 illustrates an improved object novelty preference in SCID mice. There was no significant difference in total exploration time (FIG. 35A), indicating that the motor activity of SCID mice was not influenced. Compared to the wild type, SCID mice (14 months old, FIG. 35B-C; 7 months old, not shown) spent less time in exploring the familiar object and the difference in discrimination index between SCID and wild type was significant. These results show that object recognition was significantly improved in SCID mice but their exploration activity was not influenced.

The memory improvement shown in SCID mice is not lymphocyte-related, because there was also no significant age-related change in Morris water maze performance in WT mice and Rag1^{-/-} mice (data not shown).

These results suggest that loss of DNA-PKcs activity results in a cognitive ability that is higher than WT mice in young adulthood and that continues to increase up to middle-age.

### Example 30

### DNA-PKcs Deficiency Causes Decreased ROS Production and SCID Tissues Have Lower ROS Levels

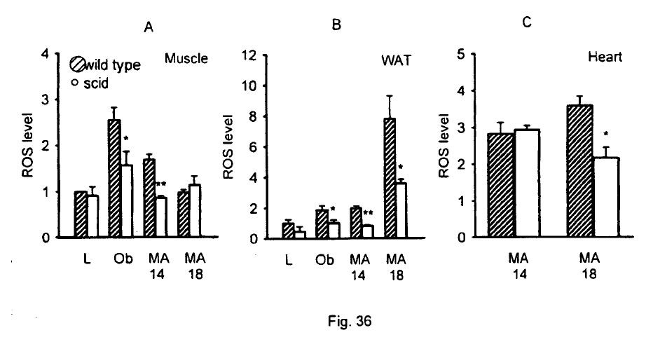

It is widely believed that reactive oxygen species (ROS) drive the aging process as well as the diseases associated with aging and a number of the degenerative diseases that can occur earlier in life. Because the DNA-PKcs inhibitor NU7026 and other compounds that showed DNA-PKcs suppression decreased reactive oxygen species production in cells, it was anticipated that SCID tissues (DNA-PKcs deficient) would have lower reactive oxygen species levels. Because uncoupling proteins and PGC-1α, which have been shown to reduce reactive species, are increased in SCID mice, experiments were performed to ascertain whether ROS is decreased in the absence of DNA-PKcs. As shown in FIG. 36, reactive oxygen species levels are decreased in SCID muscle, heart and fat compared to those in WT mice. Liver or whole brain did not show statistically significant change (data not shown).

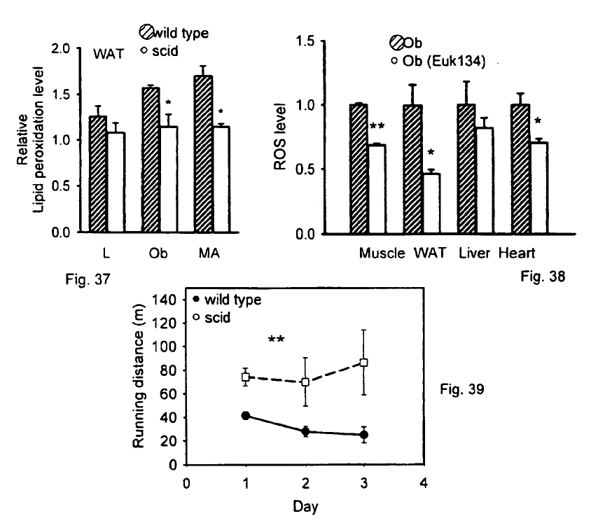

Levels of the lipid peroxidation product, malondialdehyde (Draper and Hadley Methods Enz 186: 421, 1990), were also measured as a marker of the harmful effects of the free radicals that take place in the different body tissues of WT and SCID mice. Lipid peroxidation levels in white adipose tissues were significantly lower in obese and middle-aged SCID mice compared to the WT mice (FIG. 37).

### Example 31

### DNA-PKcs Inhibitor Euk-134 Decreases Reactive Oxygen Species (ROS) Production in ob/ob Tissue

As illustrated above, the role of DNA-PKcs in ROS production is cell autonomous because treatment of MCF7 cells with DNA-PKcs inhibitor NU7026 also decreased ROS production (FIG. 4). Other compounds that suppressed DNA-PKcs activity such as DNP, Euk-134 and MnTBAP (Example 4, FIG. 4) also decreased ROS production in MCF7 cells. These results suggest that ROS-reducing property of DNA-PKcs inhibitors may be useful for treating diseases and conditions for which reducing ROS may improve the clinical course and or the outcome.

In order to test whether DNA-PKcs inhibitors would show decreased ROS production in vivo, ob/ob mice (leptin-deficient mice) were treated with a commercially available catalytic scavenger of ROS that also exhibited DNA-PKcs inhibition, Euk-134, and the ROS levels were observed in ob/ob tissues. Treatment of ob/ob mice with Euk-134 decreased ROS production in muscle, WAT and heart tissues (FIG. 38). These results indicate that DNA-PKcs inhibitors indeed decrease ROS production in vivo.

### Example 32

### Euk-134 Improves Treadmill Running Ability in ob/ob Mice In Vivo

In order to test whether DNA-PKcs inhibitors with ROS-decreasing activity would show the beneficial effects of DNA-PKcs deficiency observed in this study, ob/ob mice were treated with Euk-134, and the treadmill running ability of these mice was then tested. Euk-134 treatment increased running ability of ob/ob mice dramatically (FIG. 39) indicating that the ROS-reducing property of DNA-PKcs inhibitors indeed mimics the beneficial effects exerted in SCID mice and therefore may be useful to treat various diseases and conditions.

### Example 33

### DNA-PKcs Inhibitor Compound 36 (Cpd36) Improves Glucose Response in High-Fat Induced Type 2 Diabetes Mouse Model

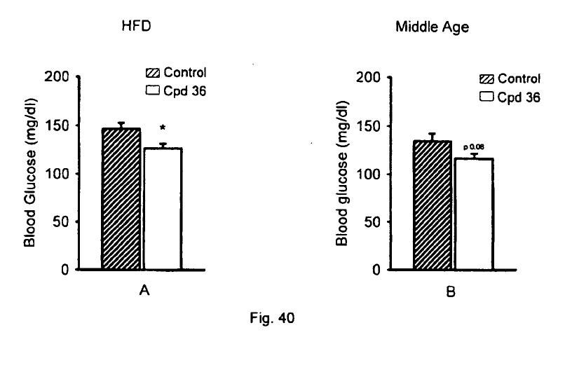

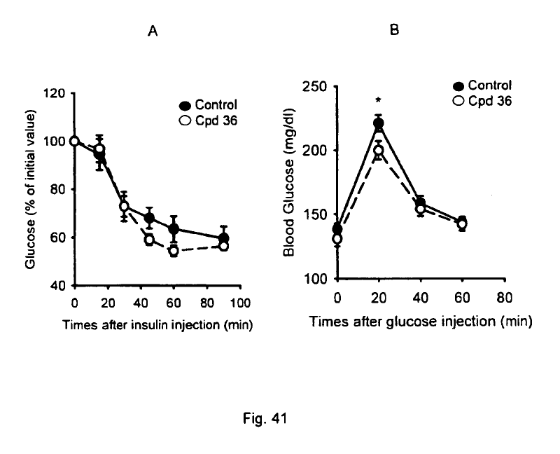

The therapeutic potential of DNA-PKcs inhibitors to treat insulin resistance and diabetes was tested in vivo using high-fat induced type 2 diabetes and obesity models. We examined the effects of DNA-PKcs inhibition by feeding C57BL6/J mice with Compound 36 (Cpd36) or vehicle. Mice were treated with Cpd36 (8 mg/kg body weight) twice daily by oral gavage for three months in all efficacy studies shown in FIGS. 40-47.

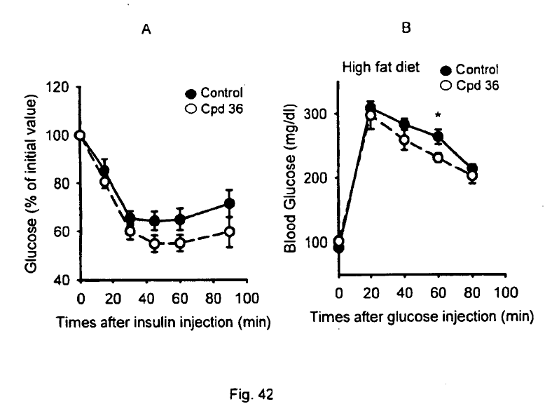

The treatment lowered fed glucose levels in the serum of both HFD (obese) mice (FIG. 40A) and middle-aged mice (FIG. 40B) (breeder diet for 13 months). The treatment increased insulin sensitivity and glucose tolerance in middle-aged mice (FIG. 41A-B). In obese mice, treatment also increased insulin sensitivity and glucose tolerance (FIG. 42A-B).

### Example 34

### DNA-PKcs Inhibitor Cpd36 Prevents Weight Gain in HFD Treated Mice

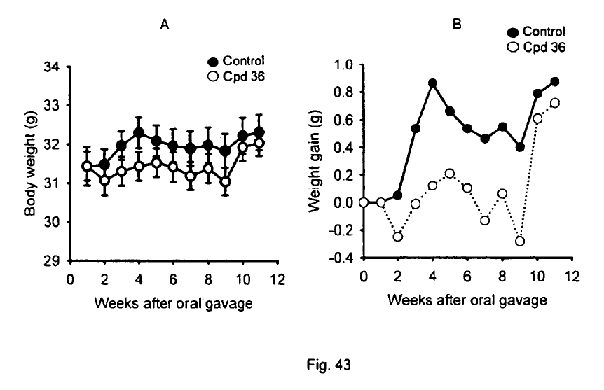

Body weight was monitored in mice receiving Cpd36 (8 mg/kg body weight) twice daily by oral gavage for three months. As shown in FIG. 43, mice did not increase in body weight substantially when treated with Cpd36, even though their food intake did increase (data not shown). This lack of weight gain was not due to increased activity levels in Cpd36-treated mice because there was no significant difference in the activity of mice treated with Cpd36 compared to the control HFD (high fat diet) mice (data not shown).

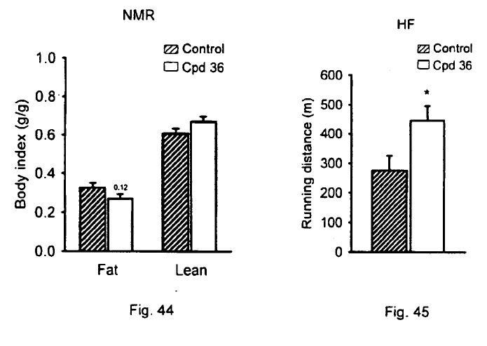

Although the body weight was not affected by the treatment, there was a tendency toward decreased fat mass in treated mice. In NMR studies (FIG. 44), Cpd36 treatment resulted in a reduction in fat mass but a slight increase in lean mass compared to the control mice after being fed a high fat diet (HFD). Thus, the treated mice ate more but did not gain weight.

### Example 35

### Treatment with Cpd36 Improves Physical Endurance in Obese or Middle-Aged Mice

The therapeutic potential of DNA-PKcs inhibitors to reverse physical decline with aging was tested. In particular, the physical endurance of obese and middle-aged mice treated with Cpd36 was examined by treadmill running. Obese mice fed a high-fat diet that were treated with Cpd36 were able to run 40-50% farther over a given period of time than the control mice fed the same high fat diet (FIG. 45), as measured by totaling the distance run on a treadmill. Middle-aged mice (13 months) fed a breeder diet who were similarly treated with Cpd36 were also able to run 40-50% farther than middle aged mice that received no such treatment (data not shown).

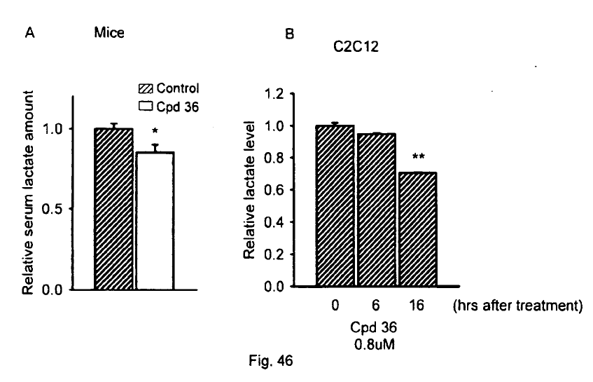

The use of Cpd36 may also facilitate more rapid recovery following intense physical exertion. Blood lactate concentrations were measured in capillary blood during a standardized treadmill test. Significantly lower serum lactate levels were observed in mice treated with Cpd36 (FIG. 46A). Reduced lactate levels were also observed in extracts of C2C12 cells treated with Cpd36 (0.8 μM) after 16 hrs (FIG. 46B).

The glycolysis pathway begins with glucose and ends with the synthesis of pyruvate. If glycolysis is to continue when no oxygen is present or in short supply as in a working muscle, pyruvate is converted to lactate. Lactate is thus a waste product. Lactate is then converted to pyruvate in order to synthesize glucose through gluconeogenesis. In cells, pyruvate and lactate interchange. Cellular pyruvate level was also increased in C2C12 cells treated with 0.8 μM Cpd36 (data not shown).

These results indicate that DNA-PKcs inhibitors may improve physical endurance through increased mitochondrial biogenesis, particularly in obese or older age animals.

### Example 36

### Treatment with DNA-PKcs Inhibitors Cpd36 and Nu7026 Decreases Anxiety Levels and Pain Sensation

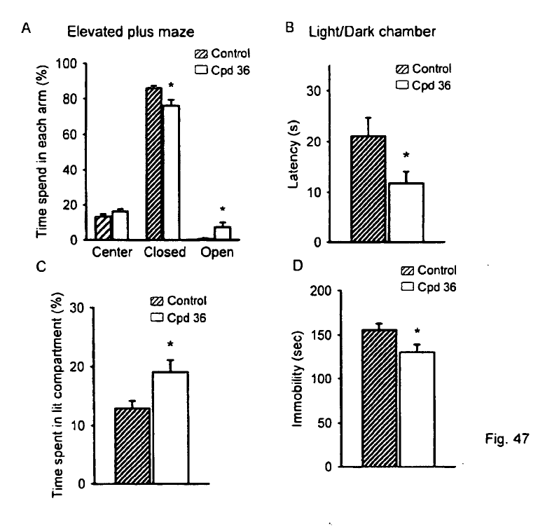

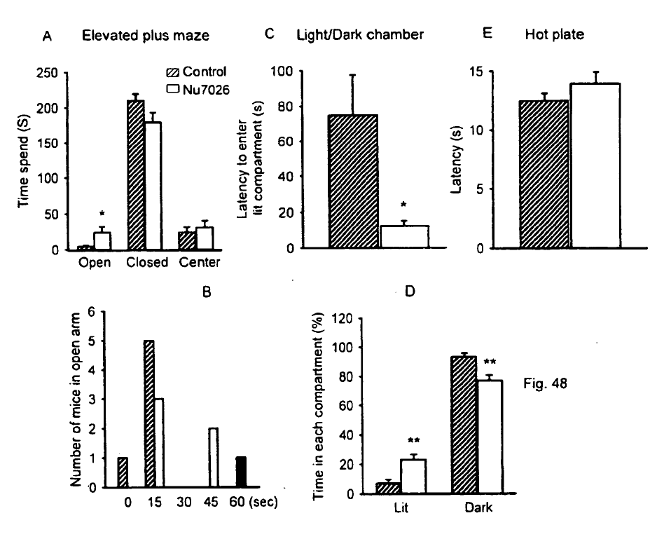

Elevated plus maze tests, light/dark chamber tests, forced swim tests (FST) and hot plate tests were performed as described earlier. Mice treated with Cpd36 or Nu7026 showed dramatically lower levels of anxiety/depression (FIGS. 47 and 48). This treatment also decreased pain sensation (FIG. 48E) and decreased immobility in the forced swim test (FST; data not shown).

The findings that Cpd 36 or NU7026 at a low-dose decreased anxiety/depression indicate that these chemicals can penetrate the blood-brain barrier (BBB) efficiently. For any central nervous system drugs, a major rate-limiting step for uptake into the brain is BBB permeability. It is known that more than 98% of all small molecules do not cross the BBB (Temsamani et al. PSTT 3:155, 2000; Jong and Huang, Current Drug Targets-Infectious Disorders 5:65, 2005). The BBB is an essential physiological barrier for the maintenance and regulation of brain function. It is comprised of brain microvascular endothelial cells that are connected by tight junctions. This transvascular route to the brain is impenetrable to the majority of drugs. Enhanced BBB permeability is obtained, for example, by increasing the lipid solubility of the water-soluble molecules. Our results suggest that DNA-PKcs inhibitors, which exhibit similar or improved levels of DNA-PKcs inhibition to those of Cpd 36 or NU7026, in particular improved solubility and/or smaller molecular weight to enhance BBB penetration, will be useful for treatment of various neurological disorders.

### Example 37

### DNA-PKcs Inhibitor Increases Mitochondrial Content and Elevates Sirt1 and PGC-1α Expression in C2C12 Myoblasts

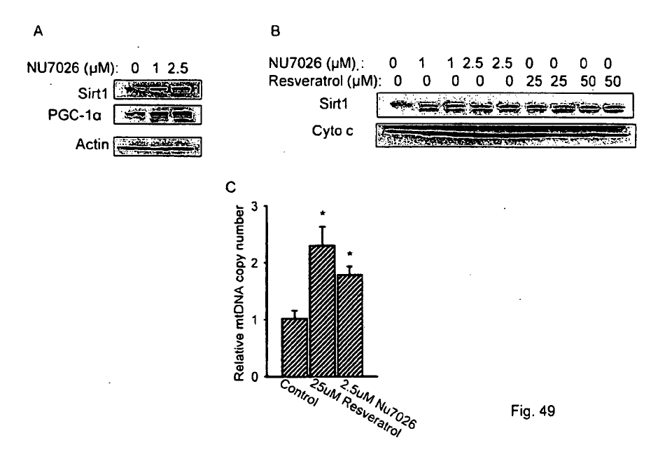

Increased mitochondrial function boosts energy production and physical endurance. As shown above, mice treated with DNA-PKcs inhibitor Cpd36 ran a significantly greater distance on a treadmill test (FIG. 45). Further tests were therefore conducted to ascertain whether DNA-PKcs inhibitor elevates mitochondrial biogenesis and expression of PGC-1α and Sirt, which are important for mitochondrial biogenesis. PGC-1α and Sirt1 expression were dramatically elevated after NU7026 treatment in C2C12 myoblasts (FIG. 49). It is important to note that only 2.5 μM of NU7026 was required to induce a similar level of Sirt1 to that induced by 25-50 μM Resveratrol, a well-known, potent Sirt1 activator. In FIG. 49C, mitochondrial DNA copy number increased (>1.8 fold) in C2C12 cells after 2.5 μM NU7026 treatment.

The results in FIG. 49 are consistent with the results obtained on SCID muscle where higher levels of expression of genes important for mitochondrial biogenesis and function were observed (Example 14). As described above, SCID skeletal muscle has an increased mitochondrial content (FIG. 15) and SCID mice run almost twice the distance of WT mice with exceptional running endurance (FIG. 17).

The results shown in Examples 14-16 and Example 37 suggest that DNA-PKcs inhibitors may also prove useful for treatment of mitochondrial disorders. Mitochondrial diseases include more than 40 different identified diseases and many mitochondrial diseases are known to be due to abnormalities of mitochondrial DNA. In these diseases, the mitochondria are unable to completely oxidize food in order to generate ATP creating energy crisis. Because mitochondria are in every organ, patients with mitochondrial diseases suffer from multisystem defects. There is currently no cure for all if not most mitochondrial diseases.

### Example 38

### AMPK Activation After Treatment with DNA-PKcs Inhibitors is Not Due to Ca²⁺/Calmodulin-Dependent Kinase Activation

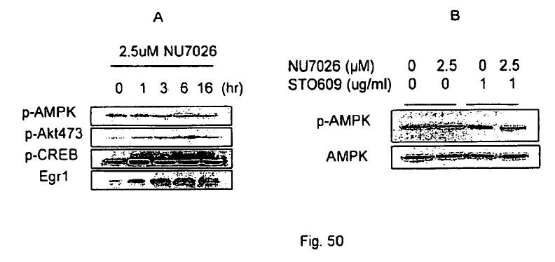

Expression of PGC-1α (FIGS. 12-14 and 49) can be induced by multiple proteins, including the energy sensor AMPK and the longevity protein Sirt1. Nutrient deprivation stimulates AMPK activity due to increasing AMP/ATP ratios. AMPK is activated by LKB1 kinase. Alternately, AMPK is also activated by a Ca²⁺/calmodulin-dependent protein kinase (CaMK) without AMP. In order to test which upstream kinase plays a major role for AMPK activation after DNA-PKcs inhibition, C2C12 cells were treated with NU7026 in the presence or absence of a known CaMK chemical inhibitor ST0609. AMPK was activated as shown in previous figures and AMPK activation was still observed in the presence of STO609 (FIG. 50). These results indicate that AMPK activation after NU7026 treatment is not mediated by the CaM kinase but may result from LKB1 activation. This raised the possibility that DNA-PKcs suppresses LKB1 and subsequently AMPK.

### Example 39

### DNA-PKcs Suppresses LKB1 In Vitro and In Vivo, and SCID Mice Have a Higher Basal LKB1 Activity

Cells were treated with DNA-PKcs inhibitors to ascertain whether DNA-PKcs suppresses LKB1 activity in vitro. Similarly, mice were treated with DNA-PKcs inhibitor and SCID (DNA-PKcs deficient) mice were observed to ascertain whether DNA-PKcs suppresses LKB1 activity in vivo.

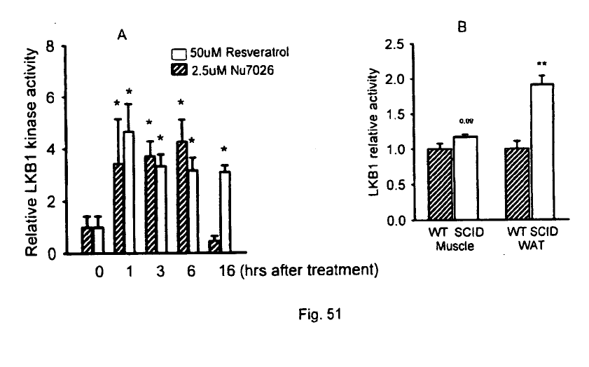

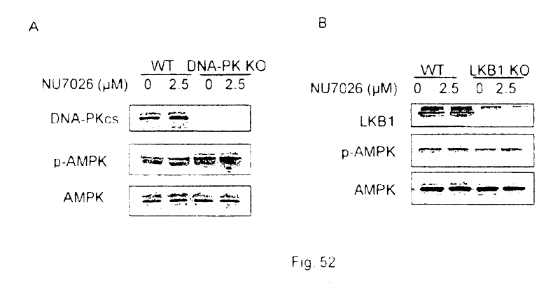

FIG. 51A shows that in C2C12 cells, treatment with a low-dose of NU7026 (2.5 μM) caused LKB1 activation as shown by LKB1 phosphorylation. Resveratrol (50 μM) also showed LKB1 activation as reported in other studies (Hou X et al. J Biol Chem 2008; Dasgupta Bet al. Proc Natl Acad Sci USA. 2007, 104(17):7217-22) suggesting that both DNA-PKcs inhibitors and Resveratrol may activate AMPK and Sirt1 via similar signaling pathways.

Mice treated with a low-dose of NU7026 exhibited a strong induction of LKB1 activity in their muscles as measured by LKB1 IP kinase assay (data not shown). Both SCID muscle and white adipose tissues (WAT) showed increased LKB1 activity indicating that SCID mice have a higher basal LKB1 activity (FIG. 51B). Together, these results indicate that DNA-PKcs inhibition/deficiency induces LKB1 activation leading to AMPK activation.

### Example 40

### LKB1 is Required for DNA-PKcs Inhibitor-Mediated AMPK Activation

The hypothesis that DNA-PKcs inhibition/deficiency induces activation of the LKB1-AMPK pathway was further tested using DNA-PK^{-/-} (null) mouse embryonic fibroblasts (MEFs) and LKB1-null MEFs treated with NU7026.

In DNA-PK^{-/-} MEFs treated with NU7026, AMPK activation was significantly enhanced (FIG. 52A). This result is consistent with previous findings that SCID mice have a higher basal AMPK activity (FIG. 18), DNA-PKcs inhibitor activates AMPK (FIG. 20) and that DNA-PKcs RNAi activates AMPK (FIG. 21). These results, in conjunction with the data that inhibition of DNA-PKcs increases LKB1 activity (FIG. 51), support the hypothesis that DNA-PKcs suppresses AMPK via suppression of LKB1.

As expected, AMPK was activated in wild-type and DNA-PK^{-/-} (null) MEFs after the NU7026 treatment, but not in LKB1-null MEFs (FIG. 52A-B). These data clearly indicate that LKB1 is an upstream regulator of the DNA-PKcs inhibitor-exhibited AMPK activation.

### Example 41

### AMPK α1/α2 are Required for PGC-1α Activation After Treatment with DNA-PKcs Inhibitor

As shown in FIGS. 12-14 and 49, DNA-PKcs inhibition/deficiency increases PGC-1α expression. PGC-1α is a metabolically beneficial protein that acts as a master mediator of mitochondrial biogenesis and function (Puigserver and Spiegelman, Endocr. Rev. 24:78, 2003). PGC-1α is activated by AMPK. In rats, swimming exercise stimulates PGC-1α gene expression in muscle (Terada S et al. Biochem Biophys Res Commun 296:350, 2002; Sriwijitkamol et al. Am J Physiol Endocrin Metab 2005). Calorie restriction also results in increased PGC-1α levels.

DNA-PKcs inhibition/deficiency induces LKB1 and AMPK activation (FIG. 51-52). Loss of DNA-PKcs function also promotes increased PGC-1α expression in SCID mice (FIGS. 14) and C2C12 cells (FIG. 49), thus it was important to investigate whether DNA-PKcs inhibitor indeed increases PGC-1α expression via AMPK in the absence of calorie restriction or exercise.

AMPK is a heterotrimer kinase composed of a catalytic α subunit, and the β and γ regulatory subunits. There are two isoforms (α1 and α2) of the catalytic α subunit. In FIG. 53, AMPK α1/α2-null MEFs were not able to induce PGC-1α expression after the NU7026 treatment confirming that DNA-PKcs inhibitor has the capacity to induce PGC-1α expression by activating AMPK without exercise or calorie restriction.

PGC-1α is also required for the induction of many ROS-detoxifying enzymes protecting neural cells from oxidative stressor-mediated cell death (St-Pierre J et al. Cell 127:397, 2006). PGC-1α null mice exhibit significantly greater sensitivity to neurodegenerative toxins. The results described herein that DNA-PKcs inhibitors induce PGC-1α expression indicate that DNA-PKcs inhibitors are also useful for the prevention and treatment of various neurodegenerative disorders.

### Example 42

### DNA-PKcs Inhibitor Cpd36 Increases Intercellular NAD:NADH Ratio

Expression of PGC-1α can be induced by Sirt1. As expected, NU7026 treatment elevated both PGC-1a and Sirt1 levels (FIG. 49). The activity of Sirt1, which is an NAD-dependent histone deacetylase, is regulated by the NAD:NADH ratios. Thus, conditions that increase the NAD/NADH ratio, such as nutrient deprivation, stimulate Sirt1 activity and PGC-1α expression.

Recent studies show that NAD (nicotinamide adenine dinucleotide) is a critical metabolic regulator of longevity, calorie-restriction mediated life-span extension and age-related diseases (Lin and Guarente, Curr Opinion Cell Biol 15:241, 2003). The benefits of calorie restriction require NAD and Sirt1 (NAD-dependent histone deacetylase) (Imai S, Nature 403:795, 2000). It is thought that calorie restriction delays age-associated diseases by regulating NAD metabolism and Sirt1 activity.

NAD serves as a coenzyme as well as a substrate for many enzymes. NAD is converted to NADH mostly by glycolysis and the tricarboxylic acid (TCA) cycle. NADH is a reduced form of NAD, and is re-oxidized to NAD mostly by mitochondria. The NAD:NADH ratio is considered an intracellular metabolic redox indicator where the NAD:NADH ratio changes in response to metabolic status (Gailward A et al. J Biol Chem 276:22559, 2001; Ramasamy Ret al. Am J Physiol 275:H 195, 1998). Previous studies have proposed that calorie restriction might exert its beneficiary effects by increasing the NAD:NADH ratio (or the NAD level) by increasing respiration to activate Sirt1. As discussed earlier in this application, Sirt1 is the principal modulator of calorie restriction-mediated beneficial effects.

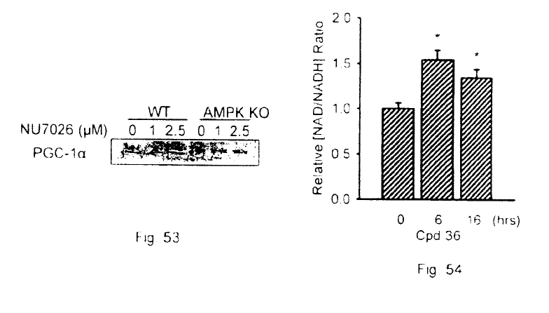

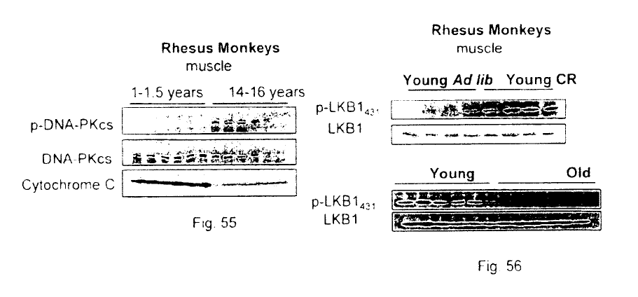

To test whether DNA-PKcs inhibitor affects Sirt1 activity, the intercellular NAD:NADH ratio was measured in C2C12 cells treated with Cpd36. As shown in FIG. 54, DNA-PKcs inhibition increased the NAD:NADH ratio. This increase was comparable to or even greater than that obtained after Resveratrol treatment (50 μM; data not shown).

### Example 43

### Aging Induces DNA-PKcs Activation In Vivo

The results described and illustrated in the foregoing Examples indicate that DNA-PKcs is activated by excess calorie ingestion or slow-energy metabolism that is coupled to ROS production. In particular, calorie restriction induces suppression of DNA-PKcs in vivo as shown in FIG. 7 (soleus muscle of calorie-restricted monkeys). On the other hand, calorie excess or obesity induced increases in DNA-PKcs expression levels (FIG. 8). These data indicate that aging accompanied by slow metabolism, elevated ROS and increased DNA damage can result in activation of DNA-PKcs.

This possibility was tested using the biopsy samples of soleus muscle of young (1-1.5 years) and middle-aged or (old) (14-16 years, equivalent to 42-48 human years) Rhesus monkeys. Among six samples tested, DNA-PKcs activity was significantly higher in at least three samples of the middle-aged or (old) monkeys than in samples of the young Rhesus monkeys (FIG. 55). Note that the level of DNA-PKcs was too low in two samples obtained from middle-aged monkeys for any meaningful interpretation. Thus, the results in this figure indicate that three out of four middle-aged or (old) muscle samples showed a higher levels of DNA-PKcs activity.

### Example 44

### Calorie Restriction Activates LKB1 While Aging Suppresses LKB1 in Rhesus Monkeys

If DNA-PKcs inhibition/deficiency that mimics the effects of calorie restriction and induces LKB1 activation leading to AMPK activation as shown in FIG. 51-52, the inventors hypothesize that 1) calorie restriction would induce LKB1 activation in vivo; and 2) aging, on the other hand, that has an opposite effect relative to calorie restriction, would cause suppression of LKB1 in vivo.

In FIG. 56, LKB1 activity was dramatically increased in muscle samples in young calorie-restricted Rhesus monkeys compared to the control monkeys fed ad lib. Moreover, the basal activity of LKB1 was significantly decreased with aging. These data suggest that calorie restriction leads to LKB1 activation while aging induces LKB1 suppression in vivo.

### SUMMARY

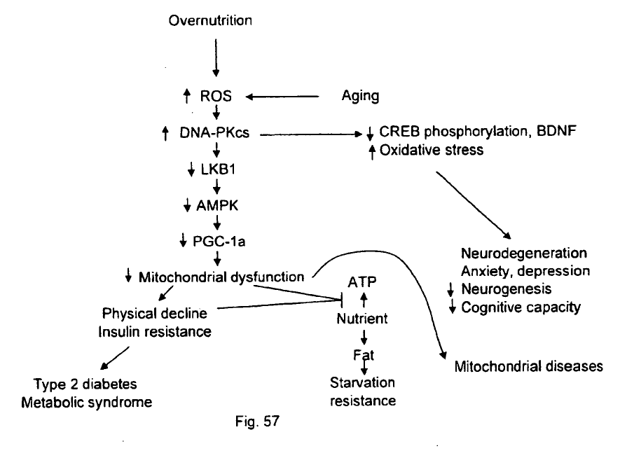

In conclusion, the inventors have identified the DNA damage sensor gene product, DNA-PKcs, as one of the mediators of mitochondrial dysfunction associated with aging and obesity. FIG. 57, shows a schematic diagram of the Stress-Activated DNA-PKcs (SAD) pathway identified by the inventors and described herein. In addition, the data shown herein demonstrates that the SAD pathway plays an important role in brain function, indicating that DNA-PKcs has a role in the mood decline, brain malfunction and neurodegenerative diseases correlated with aging.

DNA-PKcs is often activated by oxidative damage, which increases with aging and obesity. Because most reactive oxygen species are produced in mitochondria, DNA-PKcs closes the negative feedback loop by suppressing mitochondrial biogenesis. The SAD pathway may have evolved to protect cells from excessive DNA damage as well as facilitating DNA repair through NHEJ. Moreover, the SAD pathway may act as an energy thermostat to suppress the nutrient-energy conversion in times of nutritional overload. In so doing, DNA-PKcs promotes fat storage during the feast phase of the feast-famine cycle. Therefore, DNA-PKcs may be one of the “thrifty genes” (see, e.g., Ravussin, J. Clin. Invest. 109: 1537-40 (2002), specifically incorporated herein by reference, for general discussion of thrifty genes), which have been evolutionarily selected to increase survival during famine.

Improved metabolism and increased vitality in DNA-PKcs-inactive animals reveal a counterintuitive and, certainly unexpected, function of DNA-PKcs. Recently, another DNA-damage sensor, ATM, has been shown to be involved in energy metabolism. However, DNA-PKcs and ATM appear to have opposite functions in energy metabolism because ATM deficiency decreases insulin sensitivity whereas DNA-PKcs-deficiency increases it. These examples indicate that DNA-damage sensors are integral players the maintenance of metabolic homeostasis, and that they are novel drug targets for metabolic disorders. While there is no doubt that the stresses brought on by obesity and aging lead to physical, metabolic and neurological decline through direct damage of macromolecules and organelles such as mitochondria, so called wear and tear concept, the data provided herein indicate that there is also an active program that promotes this decline that involves DNA-PKcs. In particular, the obesity- and fatigue-promoting effects of DNA-PKcs promote certain ailments prevalent in Western societies such as cardiovascular diseases and diabetes.

Therefore, this invention provides new ways to reduce aging, increase energy metabolism and improve brain function. The invention also provides novel therapeutic agents and methods for treating aging, obesity-related diseases and neurodegenerative disorders. As both the incidence of obesity and the median age of the human population increase globally, the SAD pathway may play a growing role in diseases and disability.

As demonstrated herein, use of pharmaceutical compositions and therapeutic methods for inhibiting DNA-PKcs are effective ways to reverse the physical, metabolic and neurological decline, and brain and mood disorders associated with obesity or aging.

### REFERENCES

Ogden, C. L, et al. Prevalence of overweight and obesity in the United States, 1999-2004. Jama 295, 1549-55 (2006).

Bluher, M., Kahn, B. B. & Kahn, C. R. Extended longevity in mice lacking the insulin receptor in adipose tissue. Science 299, 572-4 (2003).

McCarter, R. J. et al. Physical activity as a factor in the action of dietary restriction on aging: effects in Fischer 344 rats. Aging (Milano) 9,73-9 (1997).

Kelley, D. E., He, J., Menshikova, E. V. & Ritov, V. B. Dysfunction of mitochondria in human skeletal muscle in type 2 diabetes. Diabetes 51, 2944-50 (2002).

Petersen, K. F. et al. Mitochondrial dysfunction in the elderly: possible role in insulin resistance. Science 300, 1140-2 (2003).

Short, I. R, et al. Decline in skeletal muscle mitochondrial function with aging in humans. Proc Natl Acad Sci USA 102,5618-23 (2005).

Ling, C. et al. Multiple environmental and genetic factors influence skeletal muscle PGC-1alpha and PGC-1beta gene expression in twins. J Clin Invest 114, 1518-26 (2004).

Sparks, L. M. et al. A high-fat diet coordinately downregulates genes required for mitochondrial oxidative phosphorylation in skeletal muscle. Diabetes 54, 1926-33 (2005).

Puigserver, P. et al. A cold-inducible coactivator of nuclear receptors linked to adaptive thermogenesis. Cell 92, 829-39 (1998).

Hartley, K. 0, et al. DNA-dependent protein kinase catalytic subunit: a relative of phosphatidylinositol 3-kinase and the ataxia telangiectasia gene product. Cell 82, 849-56 (1995).

Reeves, W. H. & Sthoeger, Z. M. Molecular cloning of cDNA encoding the p70 (Ku) lupus autoantigen. J Biol Chem 264, 5047-52 (1989).

Mimori, T, et al. Isolation and characterization of cDNA encoding the 80-kDa subunit protein of the human autoantigen Ku (p701p80) recognized by autoantibodies from patients with scleroderma-polymyositis overlap syndrome. Proc Natl Acad Sci USA 87, 1777-81 (1990).

Blunt, T. et al. Defective DNA-dependent protein kinase activity is linked to V(D)J recombination and DNA repair defects associated with the murine scid mutation. Cell 80, 813-23 (1995).

Critchlow, S. E. & Jackson, S. P. DNA end-joining: from yeast to man. Trends Biochem Sci 23, 394-8 (1998).

Taccioli, G. E. et al. Targeted disruption of the catalytic subunit of the DNA-PK gene in mice confers severe combined immunodeficiency and radiosensitivity. Immunity 9,355-66 (1998).

Gao, Y. et al. A targeted DNA-PKcs-null mutation reveals DNA-PK-independent functions for KU in V(D)J recombination. Immunity 9, 367-76 (1998).

Bosma, G. C., Custer, R. P. & Bosma, M. J. A severe combined immunodeficiency mutation in the mouse. Nature 301, 527-30 (1983).

Blunt, T. et al. Identification of a nonsense mutation in the carboxyl-terminal region of DNA-dependent protein kinase catalytic subunit in the scid mouse. Proc Natl Acad Sci USA 93, 10285-90 (1996).

Barlow, C, et al. Atm-deficient mice: a paradigm of ataxia telangiectasia. Cell 86, 159-71 (1996).

Short, S. C., Bourne, S., Martindale, C., Woodcock, M. & Jackson, S. P. DNA damage responses at low radiation doses. Radiat Res 164, 292-302 (2005).

Riley, P. A. Free radicals in biology: oxidative stress and the effects of ionizing radiation. Int J Radiat Biol 65, 27-33 (1994).

Chen, B. P. et al. Cell cycle dependence of DNA-dependent protein kinase phosphorylation in response to DNA double strand breaks. J Biol Chem 280, 14709-15 (2005).

Chatterjee, P. K. et al. EUK-134 reduces renal dysfunction and injury caused by oxidative and nitrosative stress of the kidney. Am J Nephrol24, 165-77 (2004).

Hollick, J. J. et al. 2,6-disubstituted pyran-4-one and thiopyran-Cone inhibitors of DNA-Dependent protein kinase (DNA-PK). Bioorg Med Chem Lett 13, 3083-6 (2003).

Ames, B. N., Shigenaga, M. K. & Hagen, T. M. Mitochondrial decay in aging. Biochim Biophys Acta 1271, 165-70 (1995).

Hardie, D. G. & Carling, D. The AMP-activated protein kinase-fuel gauge of the mammalian cell? Eur J Biochem 246, 259-73 (1997).

Faulkner, K. M., Liochev, S. I. & Fridovich, I. Stable Mn(III) porphyrins mimic superoxide dismutase in vitro and substitute for it in vivo. J Biol Chem 269, 23471-6 (1994).

Rogakou, E. P., Pilch, D. R., Orr, A. H., Ivanova, V. S. & Bonner, W. M. DNA double-stranded breaks induce histone H2AX phosphorylation on serine 139. J Biol Chem 273,5858-68 (1998).

Anderson, C. W. & Lees-Miller, S. P. The nuclear serinelthreonine protein kinase DNA-PK. Crit Rev Eukaryot Gene Expr 2,283-314 (1992).

Finnie, N. J., Gottlieb, T. M., Blunt, T., Jeggo, P. A. & Jackson, S. P. DNAdependent protein kinase activity is absent in xrs-6 cells: implications for site-specific recombination and DNA double-strand break repair. Proc Natl Acad Sci USA 92,320-4 (1995).

Jandacek, R. J., Heubi, J. E. & Tso, P. A novel, noninvasive method for the measurement of intestinal fat absorption. Gastroenterology 127, 139-44 (2004).

Lowell, B. B, et al. Development of obesity in transgenic mice after genetic ablation of brown adipose tissue. Nature 366,740-2 (1993).

Himms-Hagen, J. Nonshivering thermogenesis. Brain Res Bull 12, 15 1-60 (1984).

Kasturi, R. & Wakil, S. J. Increased synthesis and accumulation of phospholipids during differentiation of 3T3-L1 cells into adipocytes. J Biol Chem 258, 3559-64 (1983).

Uldry, M, et al. Complementary action of the PGC-1 coactivators in mitochondrial biogenesis and brown fat differentiation. Cell Metab 3, 333-41 (2006).

Nisoli, E, et al. Calorie restriction promotes mitochondrial biogenesis by inducing the expression of eNOS. Science 310, 314-7 (2005).

Wu, H. et al. Regulation of mitochondrial biogenesis in skeletal muscle by CaMK. Science 296, 349-52 (2002).

Zong, H, et al, AMP kinase is required for mitochondrial biogenesis in skeletal muscle in response to chronic energy deprivation. Proc Natl Acad Sci USA 99, 15983-7 (2002).

Hawley, S. A. et al. Complexes between the LKB1 tumor suppressor, STRAD alphaheta and M025 alphaheta are upstream kinases in the AMP-activated protein kinase cascade. J Biol 2, 28 (2003).

Woods, A, et al. LKB1 is the upstream kinase in the AMP-activated protein kinase cascade. Curr Biol 13, 2004-8 (2003).

Munday, M. R., Campbell, D. G., Carling, D. & Hardie, D. G. Identification by amino acid sequencing of three major regulatory phosphorylation sites on rat acetyl-CoA carboxylase. Eur J Biochem 175, 331-8 (1988).

Sim, A. T. & Hardie, D. G. The low activity of acetyl-CoA carboxylase in basal and glucagon-stimulated hepatocytes is due to phosphorylation by the AMPactivated protein kinase and not cyclic AMP-dependent protein kinase. FEBS Lett 233, 294-8 (1988).

Oettinger, M. A., Schatz, D. G., Gorka, C. & Baltimore, D. RAG-1 and RAG-2, adjacent genes that synergistically activate V(D)J recombination. Science 248, 15 17-23 (1 990).

Mombaerts, P, et al. RAG-1-deficient mice have no mature B and T lymphocytes. Cell 68, 869-77 (1992).

Lorenz, E., Heston, W. E., Eschenbrenner, A. B. and Deringer, M. I. The biological studies in the tolerance range. Radiology 49, 274-285 (1947).

Espejel, S. et al. Shorter telomeres, accelerated ageing and increased lymphoma in DNA-PKcs-deficient mice. EMBO Rep 5,503-9 (2004).

Berrigan, D. et al. Phenotypic effects of calorie restriction and insulin-like growth factor-1 treatment on body composition and bone mineral density of C57BL16 mice: implications for cancer prevention. In Vivo 19,667-74 (2005).

Neel, J. V. Diabetes mellitus: a “thrifty” genotype rendered detrimental by “progress”? Am J Hum Genet 14,353-62 (1962).

Bailey, S. M, et al. DNA double-strand break repair proteins are required to cap the ends of mammalian chromosomes. Proc Natl Acad Sci USA 96, 14899-904 (1999).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method of inhibiting DNA-PKcs expression and/or activity in a mammal to increase mitochondrial numbers, to increase thermogenesis, to increase insulin sensitivity, to improve insulin signaling, to reduce blood glucose levels, to increase AMPK and PGC-1 alpha activities, to improve motor function, to improve memory and learning abilities, to reduce depression and anxiety, to reduce inflammatory signaling, and/or to increase Sirt1, eNOS, VEGF and BDNF expression, the method comprising
administering to the mammal a therapeutically effective amount of an inhibitor of DNA-PKcs activity
to reduce weight in the mammal, to increase mitochondrial numbers, to increase thermogenesis, to increase insulin sensitivity, to improve insulin signaling, to reduce blood glucose levels, to increase AMPK and PGC-1 alpha activities, to improve motor function, to improve memory and learning abilities, to reduce depression and anxiety, to reduce inflammatory signaling and/or to increase Sirt1, eNOS, VEGF and BDNF expression in the mammal.

2. The method of claim 1, which reduces weight in a mammal.

3. The method of claim 1, wherein the mammal is obese.

4. The method of claim 1, wherein the mammal is middle-aged.

5. The method of claim 2, wherein the mammal reduces weight by about 5% to about 20% relative to a control mammal that does not receive the inhibitor.

6. The method of claim 2, wherein the method reduces the mammal's fat mass relative to a mammal that has not received the DNA-PKcs inhibitor.

7. The method of claim 6, wherein the mammal's fat mass is reduced by about 5% to about 30% relative to a control mammal that does not receive the inhibitor.

8. The method of claim 1, wherein serum triglycerides and/or serum leptin levels are reduced in the mammal.

9. The method of claim 9, wherein the serum triglycerides and/or serum leptin levels are reduced by about 5% to about 70% in the mammal relative to a control mammal that does not receive the inhibitor.

10. The method of claim 2, wherein the mammal does not significantly restrict calorie intake.

11. The method of claim 1, wherein the mammal can run about 1.25 to about 3 times farther before exhaustion than a mammal that did not receive the inhibitor.

12. The method of claim 1, wherein mitochondrial numbers increase in the mammal by about two-fold to about three-fold relative to a control mammal that does not receive the inhibitor.

13. The method of claim 1, wherein thermogenesis increases in the mammal.

14. The method of claim 13, wherein the thermogenesis increases the mammal's body temperature.

15. The method of claim 14, wherein the mammal's body temperature increases by about 0.1° C. to about 1° C. relative to a control mammal that does not receive the inhibitor.

16. The method of claim 1, wherein the method also increases oxygen usage in the mammal.

17. The method of claim 16, wherein oxygen usage increases by about 5% to about 20% relative to a control mammal that does not receive the inhibitor.

18. The method of claim 1, wherein the method also increases AMPK, PPAR delta, CPT1b, UCP3, ERR alpha, VEGF, Sirt1, eNOS, PGC-1 alpha and/or PGC-1 beta expression in the mammal.

19. The method of claim 1, wherein the method improves the mammal's stamina during physical activity.

20. The method of claim 19, wherein the mammal can run about 1.25 to about 3 times farther before exhaustion than a mammal that did not receive the inhibitor.

21. The method of claim 1, wherein ATP levels are higher in the mammal relative to a control mammal that does not receive the inhibitor.

22. The method of claim 21, wherein ATP levels are higher by about 5% to about 30% relative to a control mammal that does not receive the inhibitor.

23. The method of claim 1, wherein the method also reduces blood pressure.

24. The method of claim 23, wherein blood_pressure is reduced by about 10 mm Hg to about 30 mm Hg.

25. The method of claim 1, wherein insulin sensitivity and/or insulin signaling is increased in the mammal.

26. The method of claim 26, wherein insulin levels are lower in the mammal by about 10% to about 50% relative to a control mammal that does not receive the inhibitor.

27. The method of claim 25, wherein glucose levels are lower in the mammal after insulin treatment than in a control mammal that does not receive the inhibitor.

28. The method of claim 25, wherein glucose levels are about 5% to about 40% lower in the mammal after insulin treatment than in a control mammal that does not receive the inhibitor.

29. The method of claim 1, wherein memory and/or learning ability are improved in a mammal.

30. The method of claim 29, wherein the mammal remembers where a target object is located better than a control mammal that did not receive the inhibitor.

31. The method of claim 29, wherein the mammal remembers where a target object is located about 50% to about 100% better than a control mammal that did not receive the inhibitor.

32. The method of claim 29, wherein brain-derived neurotrophic factor (BDNF) expression is increased in the mammal.

33. The method of claim 32, wherein brain-derived neurotrophic factor (BDNF) expression is increased in the mammal by about 10% to about 40% relative to a control mammal that did not receive the inhibitor.

34. The method of claim 1, wherein depression and/or anxiety is reduced in the mammal.

35. The method of claim 34, wherein the mammal engages in less anxiety-related food over-consumption.

36. The method of claim 35, wherein the mammal consumes of about 20% to about 80% less high fat food.

37. The method of claim 1, wherein the mammal is resistant to pain.

38. The method of claim 37, wherein the mammal resists pain about 10% to about 40% longer relative to a control mammal that did not receive the inhibitor.

39. The method of claim 1, wherein inflammation and/or inappropriate immune responses are reduced in the mammal.

40. The method of claim 39, wherein macrophage numbers are reduced in the mammal.

41. The method of claim 40, wherein macrophage numbers are reduced in the mammal's adipose tissue.

42. The method of claim 39, wherein macrophage numbers are reduced in the mammal by about 40% to about 80%.

43. The method of claim 1 wherein heart disease is reduced in the mammal.

44. The method of claim 43, wherein the mammal is middle-aged or older.

45. The method of claim 43, wherein levels of reactive oxygen species are reduced in the mammal.

46. The method of claim 45, wherein levels of reactive oxygen species are reduced in the mammal's heart by about 5% to about 50%.

47. The method of claim 43, wherein the mammal's blood pressure is reduced.

48. The method of claim 47, wherein the mammal's blood pressure is reduced by about 10 mm Hg to about 30 mm Hg.

49. The method of claim 1, further comprising treating or inhibiting a neurological disorder in a mammal.

50. The method of claim 49, wherein the neurological disorder is Alzheimer's, Parkinson's, Huntington's disease, Amyotropic lateral sclerosis (ALS) or Friedreich ataxia (FRDA).

51. The method of any one of claims 1-50 wherein the inhibitor is NU7026 (2-(morpholin-4-yl)-benzo[h]chomen-4-one), Euk-134, Manganese (111) tetrakis(4-benzoic acid)porphyrin (MnTBAP), 2,4-dinitrophenol (DNP), a nucleic acid that can inhibit the expression and/or translation of DNA-PKcs, a chromen-4-one compound or any combination thereof.

52. The method of claim 51, wherein the inhibitor is combined with resveratrol, metformin, thiazolidinediones (TZD), Epigallocatechin gallate (EGCG), IC60211 (2-hydroxy-4-morpholin-4-yl-benzaldehyde), IC86621 (a methyl ketone derivative of IC60211), IC486154, IC87102, IC87361, Wortmannin, LY294002, or any combination thereof.

53. The method of claim 5I, wherein the nucleic acid that can inhibit the expression and/or translation of DNA-PKcs can hybridize to a nucleic acid having SEQ ID NO:2 under physiological conditions.

54. The method of claim 51, wherein the nucleic acid that can inhibit the expression and/or translation of DNA-PKcs can hybridize to a nucleic acid having SEQ ID NO:2 under stringent hybridization conditions.

55. The method of claim 51, wherein the nucleic acid that can inhibit the expression and/or translation of DNA-PKcs is a small interfering RNA (siRNA) or a ribozyme.

56. The method of any of claims 1-55, wherein the DNA-PKcs inhibitor is one or more compounds having formula I:
R₁—Ar—R₂(R₃)ₙ I
wherein:
R₁ is a hydrogen, lower alkoxy, cycloaryl, cycloheteroaryl, cycloalkyl or cycloheteroalkyl, wherein the cycloaryl, cycloheteroaryl, cycloalkyl and cycloheteroalkyl can optionally be substituted with one to four substituents selected from the group consisting of halo, hydroxy, lower alkyl, lower alkoxy, cyano, aryl, and heteroaryl;
Ar is cycloaryl or cycloheteroaryl that can optionally be substituted with one or two oxy (═O) or thio (═S or —SH) groups;
R₂ is cycloheteroaryl or cycloheteroalkyl;
R₃ is halo, lower alkyl, lower alkoxy, cyano, aryl, and heteroaryl; and
n is an integer of 0-3.

57. The method of claim 56, wherein R₁ is hydrogen, 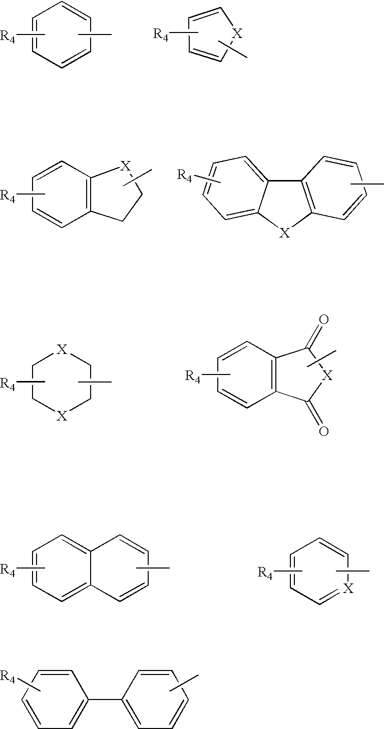
wherein X is a heteroatom, and R₄ is hydrogen, halo, hydroxy, lower alkyl, lower alkoxy, cyano, aryl, and heteroaryl.

58. The method of claim 56, wherein Ar is selected from the group consisting of: 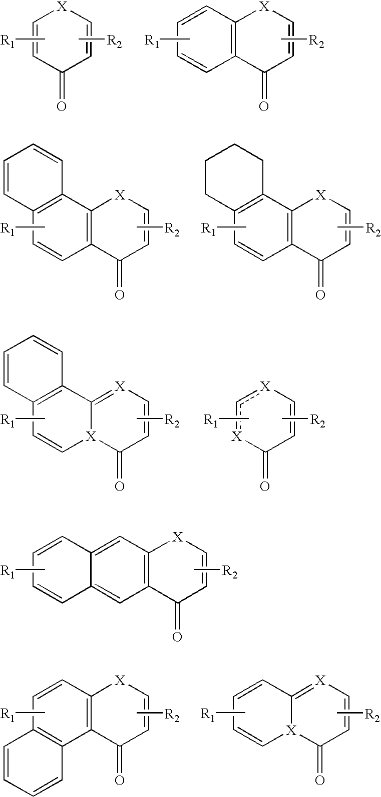
wherein X is a heteroatom.

59. The method of claim 56, wherein R₂ is selected from the group consisting of: 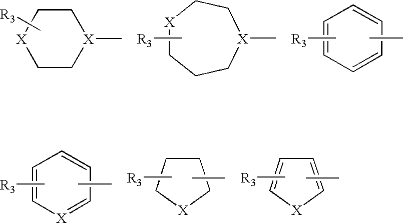
wherein X is a heteroatom, and R₃ is halo, lower alkyl, lower alkoxy, cyano, aryl, and heteroaryl.

60. The method of any of claims 1-55, wherein the inhibitor is one or more of the compounds of formula II: 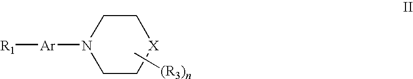
R₁ is a hydrogen, lower alkoxy, cycloaryl, cycloheteroaryl, cycloalkyl or cycloheteroalkyl, wherein the cycloaryl, cycloheteroaryl, cycloalkyl and cycloheteroalkyl can optionally be substituted with one to four substituents selected from the group consisting of halo, hydroxy, lower alkyl, lower alkoxy, cyano, aryl, and heteroaryl;
Ar is cycloaryl or cycloheteroaryl that can optionally be substituted with one or two oxy (═O) or thio (═S or —SH) groups;
X is a heteroatom selected from the group consisting of O, NH or S;
R₃ is halo, lower alkyl, lower alkoxy, cyano, aryl, and heteroaryl; and
n is an integer of 0-3.

61. The method of any of claims 1-55, wherein the inhibitor is one of the following compounds or a combination thereof: 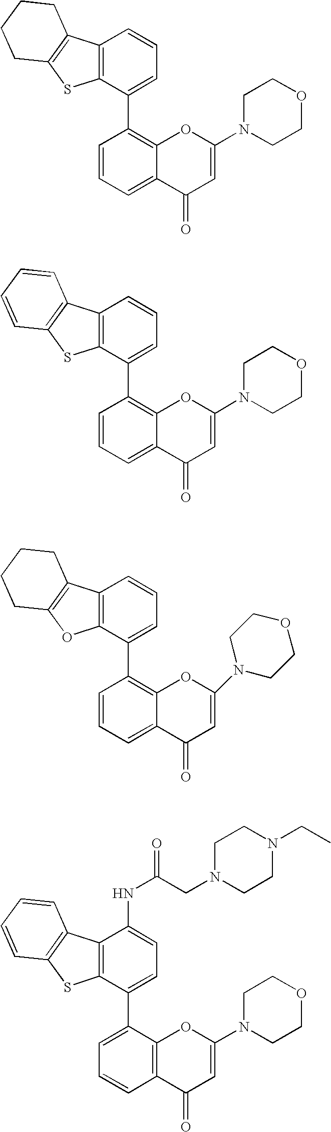 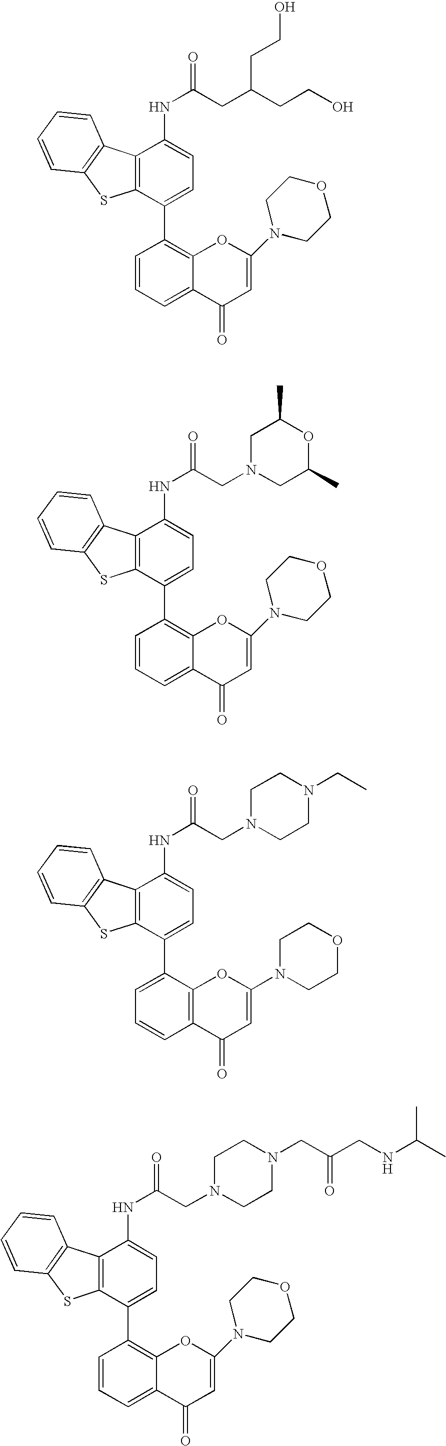 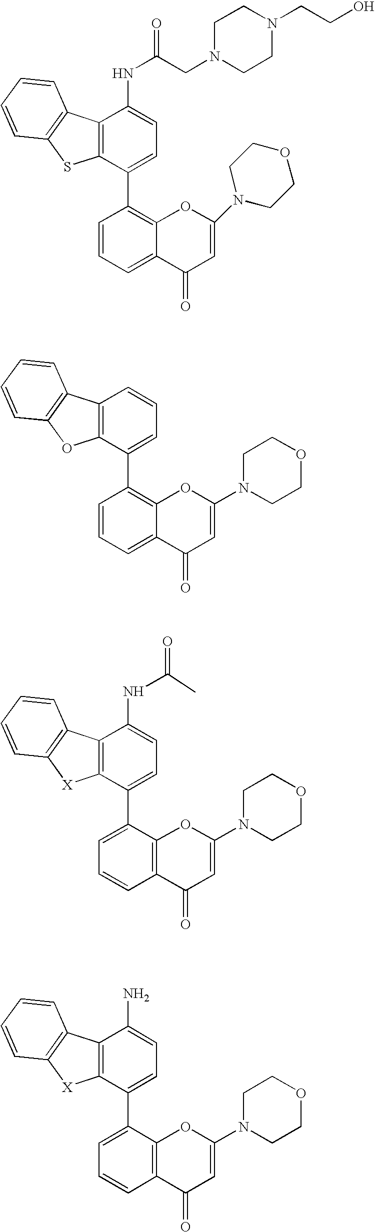 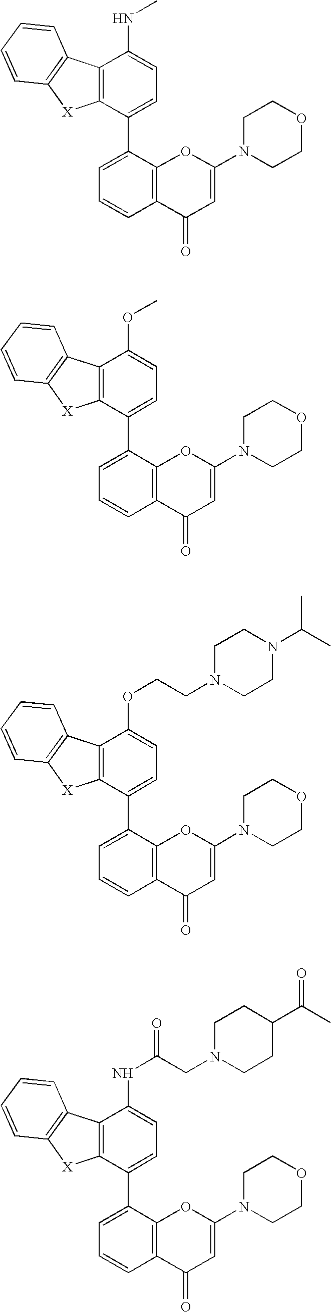 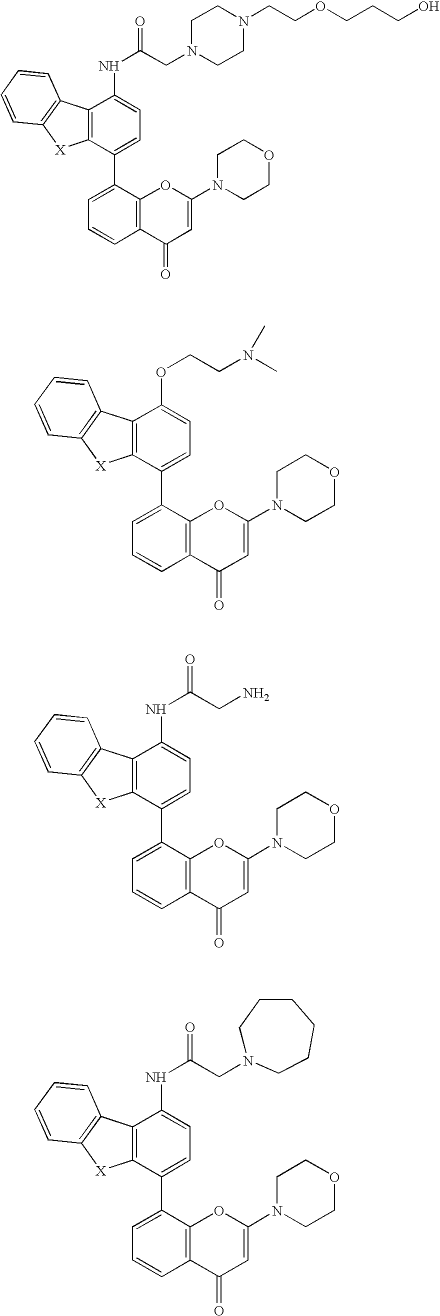 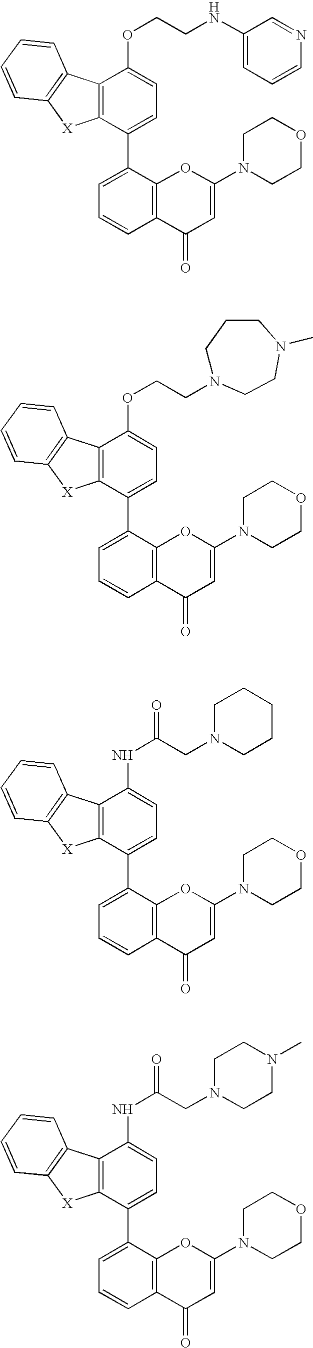 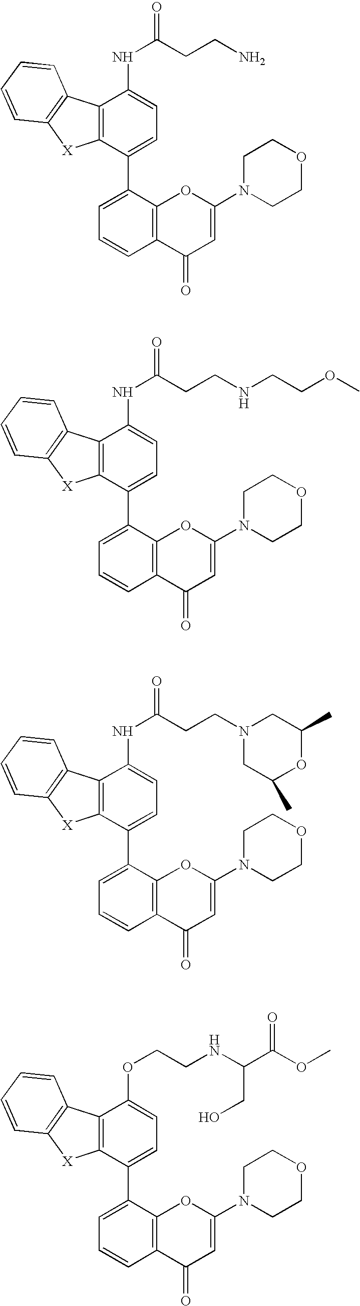 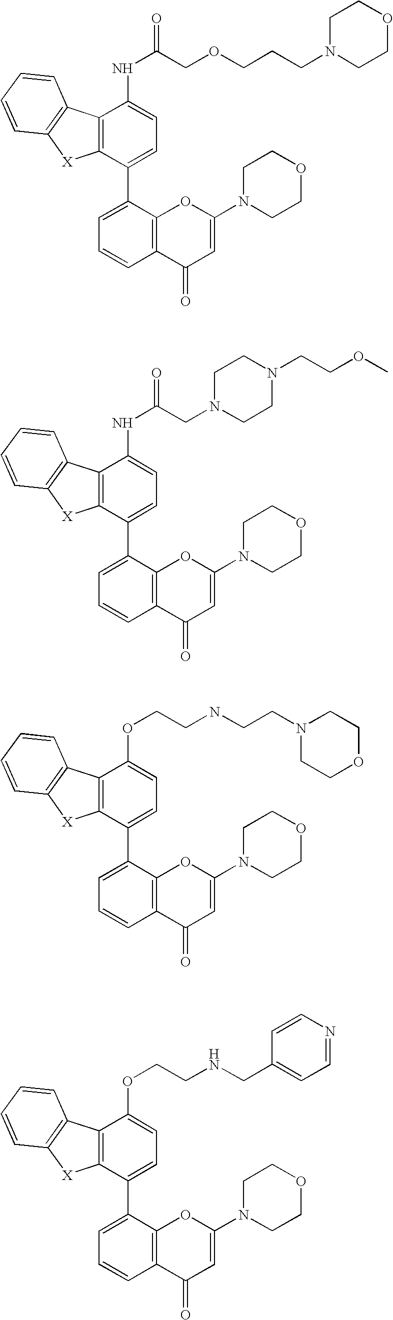 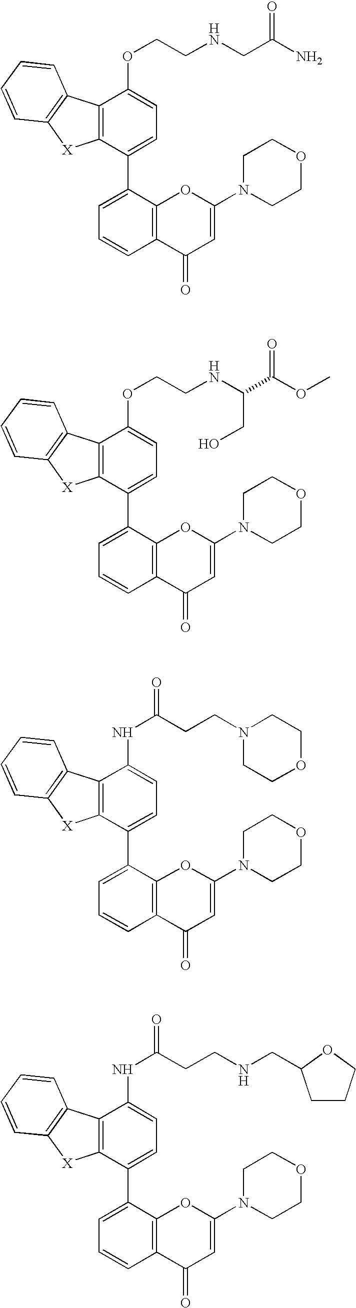
wherein X is a heteroatom selected from the group consisting of oxygen (O) or sulfur (S).


SEQUENCE LISTING (8 sequences)

SEQ ID NO: 1 - protein, 4128 aa, Homo sapiens [sequence omitted]

SEQ ID NO: 2 - DNA, 13509 bp, Homo sapiens
ggggcatttccgggtccgggccgagcgggcgcacgcgcgggagcgggactcggcggcatggcgggctccggagccggtgtgcgttgctccctgctgcggctgcaggagaccttgtccgctgcggaccgctgcggtgctgccctggccggtcatcaactgatccgcggcctggggcaggaatgcgtcctgagcagcagccccgcggtgctggcattacagacatctttagttttttccagagatttcggtttgcttgtatttgtccggaagtcactcaacagtattgaatttcgtgaatgtagagaagaaatcctaaagtttttatgtattttcttagaaaaaatgggccagaagatcgcaccttactctgttgaaattaagaacacttgtaccagtgtttatacaaaagatagagctgctaaatgtaaaattccagccctggaccttcttattaagttacttcagacttttagaagttctagactcatggatgaatttaaaattggagaattatttagtaaattctatggagaacttgcattgaaaaaaaaaataccagatacagttttagaaaaagtatatgagctcctaggattattgggtgaagttcatcctagtgagatgataaataatgcagaaaacctgttccgcgcttttctgggtgaacttaagacccagatgacatcagcagtaagagagcccaaactacctgttctggcaggatgtctgaaggggttgtcctcacttctgtgcaacttcactaagtccatggaagaagatccccagacttcaagggagatttttaattttgtactaaaggcaattcgtcctcagattgatctgaagagatatgctgtgccctcagctggcttgcgcctatttgccctgcatgcatctcagtttagcacctgccttctggacaactacgtgtctctatttgaagtcttgttaaagtggtgtgcccacacaaatgtagaattgaaaaaagctgcactttcagccctggaatcctttctgaaacaggtttctaatatggtggcgaaaaatgcagaaatgcataaaaataaactgcagtactttatggagcagttttatggaatcatcagaaatgtggattcgaacaacaaggagttatctattgctatccgtggatatggactttttgcaggaccgtgcaaggttataaacgcaaaagatgttgacttcatgtacgttgagctcattcagcgctgcaagcagatgttcctcacccagacagacactggtgacgaccgtgtttatcagatgccaagcttcctccagtctgttgcaagcgtcttgctgtaccttgacacagttcctgaggtgtatactccagttctggagcacctcgtggtgatgcagatagacagtttcccacagtacagtccaaaaatgcagctggtgtgttgcagagccatagtgaaggtgttcctagctttggcagcaaaagggccagttctcaggaattgcattagtactgtggtgcatcagggtttaatcagaatatgttctaaaccagtggtccttccaaagggccctgagtctgaatctgaagaccaccgtgcttcaggggaagtcagaactggcaaatggaaggtgcccacatacaaagactacgtggatctcttcagacatctcctgagctctgaccagatgatggattctattttagcagatgaagcatttttctctgtgaattcctccagtgaaagtctgaatcatttactttatgatgaatttgtaaaatccgttttgaagattgttgagaaattggatcttacacttgaaatacagactgttggggaacaagagaatggagatgaggcgcctggtgtttggatgatcccaacttcagatccagcggctaacttgcatccagctaaacctaaagatttttcggctttcattaacctggtggaattttgcagagagattctccctgagaaacaagcagaattttttgaaccatgggtgtactcattttcatatgaattaattttgcaatctacaaggttgcccctcatcagtggtttctacaaattgctttctattacagtaagaaatgccaagaaaataaaatatttcgagggagttagtccaaagagtctgaaacactctcctgaagacccagaaaagtattcttgctttgctttatttgtgaaatttggcaaagaggtggcagttaaaatgaagcagtacaaagatgaacttttggcctcttgtttgacctttcttctgtccttgccacacaacatcattgaactcgatgttagagcctacgttcctgcactgcagatggctttcaaactgggcctgagctatacccccttggcagaagtaggcctgaatgctctagaagaatggtcaatttatattgacagacatgtaatgcagccttattacaaagacattctcccctgcctggatggatacctgaagacttcagccttgtcagatgagaccaagaataactgggaagtgtcagctctttctcgggctgcccagaaaggatttaataaagtggtgttaaagcatctgaagaagacaaagaacctttcatcaaacgaagcaatatccttagaagaaataagaattagagtagtacaaatgcttggatctctaggaggacaaataaacaaaaatcttctgacagtcacgtcctcagatgagatgatgaagagctatgtggcctgggacagagagaagcggctgagctttgcagtgccctttagagagatgaaacctgtcattttcctggatgtgttcctgcctcgagtcacagaattagcgctcacagccagtgacagacaaactaaagttgcagcctgtgaacttttacatagcatggttatgtttatgttgggcaaagccacgcagatgccagaagggggacagggagccccacccatgtaccagctctataagcggacgtttcctgtgctgcttcgacttgcgtgtgatgttgatcaggtgacaaggcaactgtatgagccactagttatgcagctgattcactggttcactaacaacaagaaatttgaaagtcaggatactgttgccttactagaagctatattggatggaattgtggaccctgttgacagtactttaagagatttttgtggtcggtgtattcgagaattccttaaatggtccattaagcaaataacaccacagcagcaggagaagagtccagtaaacaccaaatcgcttttcaagcgactttatagccttgcgcttcaccccaatgctttcaagaggctgggagcatcacttgcctttaataatatctacagggaattcagggaagaagagtctctggtggaacagtttgtgtttgaagccttggtgatatacatggagagtctggccttagcacatgcagatgagaagtccttaggtacaattcaacagtgttgtgatgccattgatcacctatgccgcatcattgaaaagaagcatgtttctttaaataaagcaaagaaacgacgtttgccgcgaggatttccaccttccgcatcattgtgtttattggatctggtcaagtggcttttagctcattgtgggaggccccagacagaatgtcgacacaaatccattgaactcttttataaattcgttcctttattgccaggcaacagatcccctaatttgtggctgaaagatgttctcaaggaagaaggtgtctcttttctcatcaacacctttgaggggggtggctgtggccagccctcgggcatcctggcccagcccaccctcttgtaccttcgggggccattcagcctgcaggccacgctatgctggctggacctgctcctggccgcgttggagtgctacaacacgttcattggcgagagaactgtaggagcgctccaggtcctaggtactgaagcccagtcttcacttttgaaagcagtggctttcttcttagaaagcattgccatgcatgacattatagcagcagaaaagtgctttggcactggggcagcaggtaacagaacaagcccacaagagggagaaaggtacaactacagcaaatgcaccgttgtggtccggattatggagtttaccacgactctgctaaacacctccccggaaggatggaagctcctgaagaaggacttgtgtaatacacacctgatgagagtcctggtgcagacgctgtgtgagcccgcaagcataggtttcaacatcggagacgtccaggttatggctcatcttcctgatgtttgtgtgaatctgatgaaagctctaaagatgtccccatacaaagatatcctagagacccatctgagagagaaaataacagcacagagcattgaggagctttgtgccgtcaacttgtatggccctgacgcgcaagtggacaggagcaggctggctgctgttgtgtctgcctgtaaacagcttcacagagctgggcttctgcataatatattaccgtctcagtccacagatttgcatcattctgttggcacagaacttctttccctggtttataaaggcattgcccctggagatgagagacagtgtctgccttctctagacctcagttgtaagcagctggccagcggacttctggagttagcctttgcttttggaggactgtgtgagcgccttgtgagtcttctcctgaacccagcggtgctgtccacggcgtccttgggcagctcacagggcagcgtcatccacttctcccatggggagtatttctatagcttgttctcagaaacgatcaacacggaattattgaaaaatctggatcttgctgtattggagctcatgcagtcttcagtggataataccaaaatggtgagtgccgttttgaacggcatgttagaccagagcttcagggagcgagcaaaccagaaacaccaaggactgaaacttgcgactacaattctgcaacactggaagaagtgtgattcatggtgggccaaagattcccctctcgaaactaaaatggcagtgctggccttactggcaaaaattttacagattgattcatctgtatcttttaatacaagtcatggttcattccctgaagtctttacaacatatattagtctacttgctgacacaaagctggatctacatttaaagggccaagctgtcactcttcttccattcttcaccagcctcactggaggcagtctggaggaacttagacgtgttctggagcagctcatcgttgctcacttccccatgcagtccagggaatttcctccaggaactccgcggttcaataattatgtggactgcatgaaaaagtttctagatgcattggaattatctcaaagccctatgttgttggaattgatgacagaagttctttgtcgggaacagcagcatgtcatggaagaattatttcaatccagtttcaggaggattgccagaaggggttcatgtgtcacacaagtaggccttctggaaagcgtgtatgaaatgttcaggaaggatgacccccgcctaagtttcacacgccagtcctttgtggaccgctccctcctcactctgctgtggcactgtagcctggatgctttgagagaattcttcagcacaattgtggtggatgccattgatgtgttgaagtccaggtttacaaagctaaatgaatctacctttgatactcaaatcaccaagaagatgggctactataagattctagacgtgatgtattctcgccttcccaaagatgatgttcatgctaaggaatcaaaaattaatcaagttttccatggctcgtgtattacagaaggaaatgaacttacaaagacattgattaaattgtgctacgatgcatttacagagaacatggcaggagagaatcagctgctggagaggagaagactttaccattgtgcagcatacaactgcgccatatctgtcatctgctgtgtcttcaatgagttaaaattttaccaaggttttctgtttagtgaaaaaccagaaaagaacttgcttatttttgaaaatctgatcgacctgaagcgccgctataattttcctgtagaagttgaggttcctatggaaagaaagaaaaagtacattgaaattaggaaagaagccagagaagcagcaaatggggattcagatggtccttcctatatgtcttccctgtcatatttggcagacagtaccctgagtgaggaaatgagtcaatttgatttctcaaccggagttcagagctattcatacagctcccaagaccctagacctgccactggtcgttttcggagacgggagcagcgggaccccacggtgcatgatgatgtgctggagctggagatggacgagctcaatcggcatgagtgcatggcgcccctgacggccctggtcaagcacatgcacagaagcctgggcccgcctcaaggagaagaggattcagtgccaagagatcttccttcttggatgaaattcctccatggcaaactgggaaatccaatagtaccattaaatatccgtctcttcttagccaagcttgttattaatacagaagaggtctttcgcccttacgcgaagcactggcttagccccttgctgcagctggctgcttctgaaaacaatggaggagaaggaattcactacatggtggttgagatagtggccactattctttcatggacaggcttggccactccaacaggggtccctaaagatgaagtgttagcaaatcgattgcttaatttcctaatgaaacatgtctttcatccaaaaagagctgtgtttagacacaaccttgaaattataaagacccttgtcgagtgctggaaggattgtttatccatcccttataggttaatatttgaaaagttttccggtaaagatcctaattctaaagacaactcagtagggattcaattgctaggcatcgtgatggccaatgacctgcctccctatgacccacagtgtggcatccagagtagcgaatacttccaggctttggtgaataatatgtcctttgtaagatataaagaagtgtatgccgctgcagcagaagttctaggacttatacttcgatatgttatggagagaaaaaacatactggaggagtctctgtgtgaactggttgcgaaacaattgaagcaacatcagaatactatggaggacaagtttattgtgtgcttgaacaaagtgaccaagagcttccctcctcttgcagacaggttcatgaatgctgtgttctttctgctgccaaaatttcatggagtgttgaaaacactctgtctggaggtggtactttgtcgtgtggagggaatgacagagctgtacttccagttaaagagcaaggacttcgttcaagtcatgagacatagagatgatgaaagacaaaaagtatgtttggacataatttataagatgatgccaaagttaaaaccagtagaactccgagaacttctgaaccccgttgtggaattcgtttcccatccttctacaacatgtagggaacaaatgtataatattctcatgtggattcatgataattacagagatccagaaagtgagacagataatgactcccaggaaatatttaagttggcaaaagatgtgctgattcaaggattgatcgatgagaaccctggacttcaattaattattcgaaatttctggagccatgaaactaggttaccttcaaataccttggaccggttgctggcactaaattccttatattctcctaagatagaagtgcactttttaagtttagcaacaaattttctgctcgaaatgaccagcatgagcccagattatccaaaccccatgttcgagcatcctctgtcagaatgcgaatttcaggaatataccattgattctgattggcgtttccgaagtactgttctcactccgatgtttgtggagacccaggcctcccagggcactctccagacccgtacccaggaagggtccctctcagctcgctggccagtggcagggcagataagggccacccagcagcagcatgacttcacactgacacagactgcagatggaagaagctcatttgattggctgaccgggagcagcactgacccgctggtcgaccacaccagtccctcatctgactccttgctgtttgcccacaagaggagtgaaaggttacagagagcacccttgaagtcagtggggcctgattttgggaaaaaaaggctgggccttccaggggacgaggtggataacaaagtgaaaggtgcggccggccggacggacctactacgactgcgcagacggtttatgagggaccaggagaagctcagtttgatgtatgccagaaaaggcgttgctgagcaaaaacgagagaaggaaatcaagagtgagttaaaaatgaagcaggatgcccaggtcgttctgtacagaagctaccggcacggagaccttcctgacattcagatcaagcacagcagcctcatcaccccgttacaggccgtggcccagagggacccaataattgcaaaacagctctttagcagcttgttttctggaattttgaaagagatggataaatttaagacactgtctgaaaaaaacaacatcactcaaaagttgcttcaagacttcaatcgttttcttaataccaccttctctttctttccaccctttgtctcttgtattcaggacattagctgtcagcacgcagccctgctgagcctcgacccagcggctgttagcgctggttgcctggccagcctacagcagcccgtgggcatccgcctgctagaggaggctctgctccgcctgctgcctgctgagctgcctgccaagcgagtccgtgggaaggcccgcctccctcctgatgtcctcagatgggtggagcttgctaagctgtatagatcaattggagaatacgacgtcctccgtgggatttttaccagtgagataggaacaaagcaaatcactcagagtgcattattagcagaagccagaagtgattattctgaagctgctaagcagtatgatgaggctctcaataaacaagactgggtagatggtgagcccacagaagccgagaaggatttttgggaacttgcatcccttgactgttacaaccaccttgctgagtggaaatcacttgaatactgttctacagccagtatagacagtgagaaccccccagacctaaataaaatctggagtgaaccattttatcaggaaacatatctaccttacatgatccgcagcaagctgaagctgctgctccagggagaggctgaccagtccctgctgacatttattgacaaagctatgcacggggagctccagaaggcgattctagagcttcattacagtcaagagctgagtctgctttacctcctgcaagatgatgttgacagagccaaatattacattcaaaatggcattcagagttttatgcagaattattctagtattgatgtcctcttacaccaaagtagactcaccaaattgcagtctgtacaggctttaacagaaattcaggagttcatcagctttataagcaaacaaggcaatttatcatctcaagttccccttaagagacttctgaacacctggacaaacagatatccagatgctaaaatggacccaatgaacatctgggatgacatcatcacaaatcgatgtttctttctcagcaaaatagaggagaagcttacccctcttccagaagataatagtatgaatgtggatcaagatggagaccccagtgacaggatggaagtgcaagagcaggaagaagatatcagctccctgatcaggagttgcaagttttccatgaaaatgaagatgatagacagtgcccggaagcagaacaatttctcacttgctatgaaactactgaaggagctgcataaagagtcaaaaaccagagacgattggctggtgagctgggtgcagagctactgccgcctgagccactgccggagccggtcccagggctgctctgagcaggtgctcactgtgctgaaaacagtctctttgttggatgagaacaacgtgtcaagctacttaagcaaaaatattctggctttccgtgaccagaacattctcttgggtacaacttacaggatcatagcgaatgctctcagcagtgagccagcctgccttgctgaaatcgaggaggacaaggctagaagaatcttagagctttctggatccagttcagaggattcagagaaggtgatcgcgggtctgtaccagagagcattccagcacctctctgaggctgtgcaggcggctgaggaggaggcccagcctccctcctggagctgtgggcctgcagctggggtgattgatgcttacatgacgctggcagatttctgtgaccaacagctgcgcaaggaggaagagaatgcatcagttattgattctgcagaactgcaggcgtatccagcacttgtggtggagaaaatgttgaaagctttaaaattaaattccaatgaagccagattgaagtttcctagattacttcagattatagaacggtatccagaggagactttgagcctcatgacaaaagagatctcttccgttccctgctggcagttcatcagctggatcagccacatggtggccttactggacaaagaccaagccgttgctgttcagcactctgtggaagaaatcactgataactacccgcaggctattgtttatcccttcatcataagcagcgaaagctattccttcaaggatacttctactggtcataagaataaggagtttgtggcaaggattaaaagtaagttggatcaaggaggagtgattcaagattttattaatgccttagatcagctctctaatcctgaactgctctttaaggattggagcaatgatgtaagagctgaactagcaaaaacccctgtaaataaaaaaaacattgaaaaaatgtatgaaagaatgtatgcagccttgggtgacccaaaggctccaggcctgggggcctttagaaggaagtttattcagacttttggaaaagaatttgataaacattttgggaaaggaggttctaaactactgagaatgaagctcagtgacttcaacgacattaccaacatgctacttttaaaaatgaacaaagactcaaagccccctgggaatctgaaagaatgttcaccctggatgagcgacttcaaagtggagttcctgagaaatgagctggagattcccggtcagtatgacggtaggggaaagccattgccagagtaccacgtgcgaatcgccgggtttgatgagcgggtgacagtcatggcgtctctgcgaaggcccaagcgcatcatcatccgtggccatgacgagagggaacaccctttcctggtgaagggtggcgaggacctgcggcaggaccagcgcgtggagcagctcttccaggtcatgaatgggatcctggcccaagactccgcctgcagccagagggccctgcagctgaggacctatagcgttgtgcccatgacctccaggttaggattaattgagtggcttgaaaatactgttaccttgaaggaccttcttttgaacaccatgtcccaagaggagaaggcggcttacctgagtgatcccagggcaccgccgtgtgaatataaagattggctgacaaaaatgtcaggaaaacatgatgttggagcttacatgctaatgtataagggcgctaatcgtactgaaacagtcacgtcttttagaaaacgagaaagtaaagtgcctgctgatctcttaaagcgggccttcgtgaggatgagtacaagccctgaggctttcctggcgctccgctcccacttcgccagctctcacgctctgatatgcatcagccactggatcctcgggattggagacagacatctgaacaactttatggtggccatggagactggcggcgtgatcgggatcgactttgggcatgcgtttggatccgctacacagtttctgccagtccctgagttgatgccttttcggctaactcgccagtttatcaatctgatgttaccaatgaaagaaacgggccttatgtacagcatcatggtacacgcactccgggccttccgctcagaccctggcctgctcaccaacaccatggatgtgtttgtcaaggagccctcctttgattggaaaaattttgaacagaaaatgctgaaaaaaggagggtcatggattcaagaaataaatgttgctgaaaaaaattggtacccccgacagaaaatatgttacgctaagagaaagttagcaggtgccaatccagcagtcattacttgtgatgagctactcctgggtcatgagaaggcccctgccttcagagactatgtggctgtggcacgaggaagcaaagatcacaacattcgtgcccaagaaccagagagtgggctttcagaagagactcaagtgaagtgcctgatggaccaggcaacagaccccaacatccttggcagaacctgggaaggatgggagccctggatgtgaggtctgtgggagtctgcagatagaaagcattacattgtttaaagaatctactatactttggttggcagcattccatgagctgattttcctgaaacactaaagagaaatgtcttttgtgctacagtttcgtagcatgagtttaaatcaagattatgatgagtaaatgtgtatgggttaaatcaaagataaggttatagtaacatcaaagattaggtgaggtttatagaaagatagatatccaggcttaccaaagtattaagtcaagaatataatatgtgatcagctttcaaagcatttacaagtgctgcaagttagtgaaacagctgtctccgtaaatggaggaaatgtggggaagccttggaatgcccttctggttctggcacattggaaagcacactcagaaggcttcatcaccaagattttgggagagtaaagctaagtatagttgatgtaacattgtagaagcagcataggaacaataagaacaataggtaaagctataattatggcttatatttagaaatgactgcatttgatattttaggatatttttctaggttttttcctttcattttattctcttctagttttgacattttatgatagatttgctctctagaaggaaacgtctttatttaggagggcaaaaattttggtcatagcattcacttttgctattccaatctacaactggaagatacataaaagtgctttgcattgaatttgggataacttcaaaaatcccatggttgttgttagggatagtactaagcatttcagttccaggagaataaaagaaattcctatttgaaatgaattcctcatttggaggaaaaaaagcatgcattctagcacaacaagatgaaattatggaatacaaaagtggctccttcccatgtgcagtccctgtccccccccgccagtcctccacacccaaactgtttctgattggcttttagctttttgttgtttttttttttccttctaacacttgtatttggaggctcttctgtgattttgagaagtatactcttgagtgtttaataaagtttttttccaaaagta

SEQ ID NO: 3 - protein, 1689 aa, Homo sapiens
MAGSGAGVRCSLLRLQETLSAADRCGAALAGHQLIRGLGQECVLSSSPAVLALQTSLVFSRDFGLLVFVRKSLNSIEFRECREEILKFLCIFLEKMGQKIAPYSVEIKNTCTSVYTKDRAAKCKIPALDLLIKLLQTFRSSRLMDEFKIGELFSKFYGELALKKKIPDTVLEKVYELLGLLGEVHPSEMINNAENLFRAFLGELKTQMTSAVREPKLPVLAGCLKGLSSLLCNFTKSMEEDPQTSREIFNFVLKAIRPQIDLKRYAVPSAGLRLFALHASQFSTCLLDNYVSLFEVLLKWCAHTNVELKKAALSALESFLKQVSNMVAKNAEMHKNKLQYFMEQFYGIIRNVDSNNKELSIAIRGYGLFAGPCKVINAKDVDFMYVELIQRCKQMFLTQTDTGDDRVYQMPSFLQSVASVLLYLDTVPEVYTPVLEHLVVMQIDSFPQYSPKMQLVCCRAIVKVFLALAAKGPVLRNCISTVVHQGLIRICSKPVVLPKGPESESEDHRASGEVRTGKWKVPTYKDYVDLFRHLLSSDQMMDSILADEAFFSVNSSSESLNHLLYDEFVKSVLKIVEKLDLTLEIQTVGEQENGDEAPGVWMIPTSDPAANLHPAKPKDFSAFINLVEFCREILPEKQAEFFEPWVYSFSYELILQSTRLPLISGFYKLLSITVRNAKKIKYFEGVSPKSLKHSPEDPEKYSCFALFVKFGKEVAVKMKQYKDELLASCLTFLLSLPHNIIELDVRAYVPALQMAFKLGLSYTPLAEVGLNALEEWSIYIDRHVMQPYYKDILPCLDGYLKTSALSDETKNNWEVSALSRAAQKGFNKVVLKHLKKTKNLSSNEAISLEEIRIRVVQMLGSLGGQINKNLLTVTSSDEMMKSYVAWDREKRLSFAVPFREMKPVIFLDVFLPRVTELALTASDRQTKVAACELLHSMVMFMLGKATQMPEGGQGAPPMYQLYKRTFPVLLRLACDVDQVTRQLYEPLVMQLIHWFTNNKKFESQDTVALLEAILDGIVDPVDSTLRDFCGRCIREFLKWSIKQITPQQQEKSPVNTKSLFKRLYSLALHPNAFKRLGASLAFNNIYREFREEESLVEQFVFEALVIYMESLALAHADEKSLGTIQQCCDAIDHLCRIIEKKHVSLNKAKKRRLPRGFPPSASLCLLDLVKWLLAHCGRPQTECRHKSIELFYKFVPLLPGNRSPNLWLKDVLKEEGVSFLINTFEGGGCGQPSGILAQPTLLYLRGPFSLQATLCWLDLLLAALECYNTFIGERTVGALQVLGTEAQSSLLKAVAFFLESIAMHDIIAAEKCFGTGAAGNRTSPQEGERYNYSKCTVVVRIMEFTTTLLNTSPEGWKLLKKDLCNTHLMRVLVQTLCEPASIGFNIGDVQVMAHLPDVCVNLMKALKMSPYKDILETHLREKITAQSIEELCAVNLYGPDAQVDRSRLAAVVSACKQLHRAGLLHNILPSQSTDLHHSVGTELLSLVYKGIAPGDERQCLPSLDLSCKQLASGLLELAFAFGGLCERLVSLLLNPAVLSTASLGSSQGSVIHFSHGEYFYSLFSETINTELLKNLDLAVLELMQSSVDNTKMVSAVLNGMLDQSFRERANQKHQGLKLATTILQHWKKCDSWWAKDSPLETKMAVLALLAKILQIDSSVSFNTSHGSFPEVFTTYISLLADTKLDLHLK

SEQ ID NO: 4 - DNA, 9 bp, Artificial Sequence
  A synthetic oligonucleotide
uucaagaga

SEQ ID NO: 5 - DNA, 21 bp, Artificial Sequence
  A synthetic primer
ccgcaagggaaagatgaaaga

SEQ ID NO: 6 - DNA, 20 bp, Artificial Sequence
  A synthetic primer
tcgtttggtttcggggtttc

SEQ ID NO: 7 - DNA, 24 bp, Artificial Sequence
  A synthetic primer
gccagcctctcctgattttagtgt

SEQ ID NO: 8 - DNA, 24 bp, Artificial Sequence
  A synthetic primer
gggaacacaaaagacctcttctgg